SEQUENCE LISTING (44 sequences)

SEQ ID NO: 1 - DNA, 33901 bp, Homo sapiens
  source 1..33901: mol_type = genomic DNA
gcgccactgcagctggcattggccgggactggaagtgcgggcttctgcagcagccgaagctggagctgctagggtgcgaactgccagggcaggtgtgggctgcgggaggctactcgggccgggagcgccgggacaagtgcatggaggtggtgcgactcctgcaagttgctggggcagtcgtgtcccctattccctgacacccgagctccggtcttctccctgagaaatcggtcgggctcgtgcaggggtggggatcaggattcatctctcctggggagactgcgctggggcatgggttcccctgggttggaaggagtccttatggactcagtcccctaagtatctgcacgcgcggaggcgccgccagccagcccgcctgggtccctagggaacattgaggcagtaaggggtactctgagagcagccggtatgctggagataattaatgacagcgttaccattgattggactgagcgctcactgtgtgttacacactgtggtagaccctttgtttatattattccgtcctctcaacaatccaggaggttaagagctatcattctgcccattttccagatgggaaaactaaggcatagaagttaaccaacttatggtaataaaaaatcacacagatggtaagcagtggaagatttaaacctcacagctggtcttccagcctctgtccttttcagatcctctagtcagcttctctcagcgcatgctgctggctggtgcctctgggatattgcttctttattactccttgacctagagcgaactgtgcggagggaggaagtgcaaaacgtggacctcctgttggcacaaacgagcctctttgtcctctcctcatcctctcttcacttcattttctcaggcagcagcggctcccctgttgtatggacattctgcacccgaaactgatagctgagtcctgaagttttatgttatgaaacagaagaactttcatcccaggtgcattggtttattctctcccctagcctgttatgttagattgtacttatgttaaatttttaaggacataggtggttatgccgagaataacagtgactgtagccaacccatgtggggattgtaaagaaattggaatatgccttccaaaggaacactgttaatctagacatgacctttcaggattttaaaggaataattttaggccaggtgagaaacagccaaaagcaaaaataatgtgaggctagagattatgtttgaaagtttttatattttggattaatatctattctaggagtttactcttagtgaatctttttccttagggtagaaaaaagcttatcgaaaaggatgaaaacttatacatcagatatttcaaaatagtgtgcttttctttcagagctttgtagctttgtattacctttgtattaaaagtgaaaatgttcttttaggacatcacaagaaaattaagatcttaatttaagtacgggaacttaaggggtgccctaattgccttaattaaataaacatgccttccttctgccaggaactattcaattaacaaaaatgaaataagaataagctcttattttcatttgcaaatgactattcatagctattttgtttggatacaattcatagctattttgtttggatactattttgtttgcatactatgttttgtttggatactattcatagctattttgtgtggatacaaaggatacaatgttgtatccttatatattcaggaagtcttagaatacaaatgctgattttttttttttctagtcagggaagcctaactgtgttcatataaacacatcaaagtcagtttttaccttgtgactcaacatggtagaagtctgtcagttcatgtgatatcccattgtggagcaaagtaatgtagaaatcagaattttgagttctagtatttactctctcgattccttgttaatttaaatggtacctattttttatagcacatgatttgggaattacactttgtgacatggatgaatctgcactgacccttggtacaatagatgtttcttatctgccacattcatcagaatacagtgttggtcgatgtaagcacacaagtgaggaatgggtaagtttaggataagagttaaaagcactcagcctaaaaatgttcatttcaatgatgtttatgaaggtgcaaatactggcagcaacataatatcacatcaagattaatagctacagaaactggtatgtttatgaagcctaataaaaaattttatggcattatattgggaagaaaaaatggttagaaaatttatgtacactgattatgttagaaaacaaaataatcattttgcataggaaaaaaaccctagtagtagttttgggtcggtaagaaatttttttttctttctttctcttggtatgtactttccatgagttatacttgaaaagcatgaattttataatagaagtaaactttaaaaaattaggaacttatagtatgaattcaatccaaaaggcgtacattttcttggattgtttttcctatttcaatataacatgacgtattttaaatatcttaattgtctttatataaaaaatggtggggtttttttttttgcttaattttgagaatgttatatctgtataacagagatgtgagcaactgagctcaaagttaagcatttttagaatagaagagcaaggtttgaccctaattgaatagtatagctataatttaagttctggtctttaagttcccttgataagataaaggaatgttatataagtttacctgttttttatttaaagtaaaattataaaaacttgctcactatgtaaggcctttatagttctgtagtgcgactgtgtgatttaaaagggggtaaggttaattgttagactagagatccaggagtctgaattgatttatctagctgtcagaagtctgtttgaccttctacaatttagtttttaaatttttgaatactgccttcagtgtgtagctattactatccttgggtgctcagcttttgcttatgtggtaagcttttatccaagtgttgatcatggagggagtacttaaatgaaaatcacccagaatatgccataagtttttcttctgttcaaaatactttagattttgagatcaagtagacaagtattctcttgccatttagaaactatatttttttcttaatatttgtcagggtgagtgtggctttagacccaccatcttcagatctgcaactttaaaatggaaagaaagcctaatgagtcggaaaaggccatttgttggaagatgttgttactcctgcactccccagagctgggtaagaaccttactgcttattgaccctaatactcagcataaaatattatttccaactttgtagtaggcagtcttattggactaaattcactttcagaacagagacccatgtttatgaaccaaatatttcaagtaaagatgttgacacactttttctttccatttatttattcagtaaatattggtcccataattgatactaggtaacattctagttcttttgaggttttagtaggattttaagggggagagtagatcttacttttggttaagtcttcaaaggagttagtgggaaccttcatgaacttttaggtgttgaactttagcacttaacattaaaattctagcaagataagaaagagataatgggacagaataagaagttaaagctgtgactttgctctgagcctttgctaatggaaggtgtctgttgactttgtgatagtgaaagattgtcactaaaatgttagaatgaagatttaagagagtgtatatgggtttgttgtttgtattttggtgaaaaaacacaagccatgcaatatcttttgcattacagtgcagtctttgagaagagccaagtgggtgagaggtatattttcggtggtagttgaagagaaggacaaattagcacaggaacaagaacttcacgtagttgtgtttgaaggcagtagaattgccttttaaaagtcatatctggatgttaagctctctctgggatccagttattaggatgaagaaattctgccgtttaagtgcctgccatttatagaggttgcttgtaacttgtgtggctaggtaattgtgctgtgtgaattttctactcaaggttggtttggcagaaagtagaattctgagtctggtataaaggggtttactaacatgggagagatttgtgtggaacccaagcagtttatgttaaatgttcagatctgctgaagaatgtcttgttataaatattgcttcttttaaaccacaactaccttgttgcaagaatttggtagaattcaagaatcacagaaatttctagcctaggagtaaagctagttcagattaataggtcacttgaaatcaagatttaaacagaatgtcattttctttttagatctgacatctagtctaattctaattttgaatacttaaaacaataactacaatgtgttcttaagtcagagggtataggaaataatttggtggtggtaatggtttggagtaaagggtaaaggtagatagtacaacagacaattgatttttgccctactatagtacctgatggagttgaacttgatcttgaattacttccatgacatgatcatttaaaataatttgtttaaaaatgtttttattggtaactgtataatatttgctatcttttctctgaaatcgtattttccttttctctaaagagattccacttaagtagttttatatttgttgagggaacattgtgctttttcagcatttaagtgtggaacagttacctttttgcccatattgataaagctatagactatcttttcagcaaagctatctctggattatctttcaagatcacatgtaaagagggtgaccagtagacagacgatttctcattaaatggacagttatttaggggtaatagaaagaccatactctttagatcaagacataagattgaatcctaaacctctcccggtcttagtttctcacctagaaaatggtgataactcttttgtgtcagggattttgggagatttagtaagcaacaataactgcatggtacttagcgtggtctttgtattttgggaaaactgaaatctctgacattcttgtagcttcacttctttcagggtggagatgaggagtcaagcagcaaggggtactgtttcctttggttttaaggtggtttgtttgtttgtttgttttttgtttttgtttttgtttttttgagatggagtcttgctttgtcgcccaggctggagtacaatggcatcgtgttggctcactgcaacctccgtctcttaggttcaagcaattctcctgcctcagcctcccaagtagctgggattacaggcaccgaccaccacacccagctaatttttatatttttggtagagacggagtttcaccatgttggccaggctggtctcaaactaatgacctcaggtgatctgcccgcttcggcctcccaaagtgctgagattacgggtgtgagccactgcgcctggctagttttaagattttaaagatgaattttgagtttatgacattctttgaaggctaaaaagtacttttcaataaaacttttttcttccctaagatttttgaggcaatatctgtttttatgcttgatattaaagttatcaagaactcataatgagagctcttcaccgtcaatctagttgtgtatttatgtctacatttactttgttttatataggacaaatttttcaaccccagtatcccgtctttgggtttgcggaatgttatttatatcaatgaaactcacacaaggtaaaaagaaatttcttatgtttttaatacatctcaaaagaaaaacttcataataatcagttaacacactttaagctgaattgctttgcattactgagagtgaatagaatatgagtttctgacacaagagagattatctgaaaattggaagcagtatagccacagagagaactattactgttaccacttgtgtacttacagataatttcaggggagtgtagatgtcacttagaatgttctttgacttatattgatttgcatatgaagtatgccaaaatgtgggaaatatttgtctgagaagaccggtaggtttgaatctcttatgatttattatgactatttaatacttaaaaaaagtataaattattgaaatctgcccaatctgtagggtgttaaaagacattttctctgttgagcaaatgttgtttgagttactatttacccaagactttacttagagttcttacaatttctaggcatatggggataggcttgtaagcaccagtgtgagagaaaaaggtctttgttttatctgactctaggccaggggacagggaaggagggtcgaaaggttgtgtgaatgtctgtgaggcaagagaatcagtagccaaatgctagcagtccttgtaattctaaaaatagatacctggagtaggcaggcagataggctcagatgagaaatacttgaaattctatgaaaattagtgcaagaaatcaactacttgaagcccttatgatattacttctcataatttgatgatgagatcacatatgagaatgataatacttggatatatagtaaacttttaaaaatagaacttaagtctaagtacatgagcagatcattcccaggctgctggtctctctctctctctcttttaaatttgcatttaaatttaaatttaaaaaagaggggagagactttggtccgttgaattttttttgataagatgataagatcctaagaaaagggatctcagagtcttccttatgtaagtagatattttatatataaagccaagaggtttgtttcttgccctcaattagagacagttaaggaaaatcgcatttgctatgatgacgaggcagaatgaaactattcagtgaaataagctgcatgttcagctatttacttcttctgtgtattctatgaagcaaagcctaaaacatttttacacattcacatagagtcacacactctgtcactgtcacactcatgcacatgtgcgcacacacacacaatgctttcagtcagttgcaaagggatttcatagagagcttggactttgtacataccaataatatttctgaaaatgttgactgaaggaggagtttaggattaacatttaatatattgggccaaatttttggaagatttagacaaaggaagggaggaagaaaaagagaatggcaggaaggagaggtgaagaaataggtgattcataaacagttctctttcaagttactttttctctattgagggtattctgggtcttttcagagttactgaattgataataataatgtgatatcatgtaatcattacaaatgttttcaaagtattacataatgaaagcttgtagcattcttattctgaaactcatctctccatcctcccatctaaacaccacatagtcatttataactgacaggaatatgttctatccctcaggtgtatttaaaatgtgaagggggaaaaagtgagaaattctggcttcgttagcctagagtacaaatatttagatcatgcttgacttctttcaactgatctgccttgtcaattcaattagagaatcagtgattatcttaggaaaaaacaaacaaaaacttaacaaaaaaaattagtctttgatcataggaaaagacttccaaatttatctttttatagcccatagctataaatataagctgtatggtttatcagaatagatatagcttatatatagcattaaacatttttgcctttttaatggtattaataaagccagatgttcttatttcttttgttttcatattagggttattcggcacagtaacactattcaaacattaatacacaaattccaagtttcagttttagctgagagtacatgaataaaattggctgcacaacattctgaactactaaggagaatttgctgtaggagaaccctaaatctcagaaagagatgacattaggctttgtttttatgggcggaaaattagtttatgatttaagcaacttgttttaggggtacctctaatacttctctgctaaatcattttcacagacaccgcggatggcttgcaagacgcctttcttacgttctttttattcaagagcgagatgtgcataagggcatgtttgccaccaatgtgactgaaaatgtgctgaacagcagtaggtaagggcggggcaaggtgtaaaggtgggaagaggcagggatgggcggtaggagtgggagggggaagtaattcttagtaacctttagtttctctgggactgtgtctcaagaagtgttcacatttccaaacattaaatgttaagctccaaaggaaactaaagaaaggcaggagaacaggtgctttggtgacagtgccatgtgggaatagcagccttgttttatggattaataattgaatgggtttctaaaacatgaaaggcaaataagagttcagagatgcacatatttccagagttctctgctttctgtaatataaatgttggagacattgtatttcctattttatagccgaaagagaattgagtttgatatttttgccagttactgagtatctaggtacaaattcttgtactggaaagtcattgctagaaatcttaacagaatgaattactgcctttgctcttacaggagcttgcttacaataggaaagactgattggaactcaggtggaatggggaaataaatattctagaatatgggagtttgggtgaagatgttccaaactcccgtactctagaatattgattctagagtactggcctcattggtgtggccagtttcagagatctacctggatttggggagcaggagaccagggtgaattaaaaggctgctttaaattcataagtgtcatctgtagactcttcgttatgtttgttaaggcctgaacaatgctggcacataatatgtactcaagaaatacttctcgaatggatgagtgagtagttgataatggaggtgcatcagtcatggtaatgacgtgtagaaacattcctggtcagaatgcaatgaatgctttttagggaatagacttggggggcatcagtgtcctgtggtattggtctttttgtattagtgaattatggggctatgtggaattttgctgtacttcactagtccaaaatcagcaccactgggataaaacacaattatgtgaaattcttagattattatatattctgttttcttgttaaaaccttcaagtacatttatttcatccagatgttttgactagtaactgggatgttagaagtggtttaaggaggttgaacctcatgctttctcccctaagtcagctggaagatttataaaactggattaaatgtttccttttatctcactgcttattattctgtgctgtgctcttttgatcttgagctgtgccttttaaaaattactggtttgagttgaaaaaatttgtaaatatttatgaccatttttggtccctcccagagttttacatatagttttactaatggagtcacaatacaaattaaaagtaaattaagtcttcaaaagaaaaatcacacattaatttggtagtagacttttggatacctctgaattatatgcctaaagggataacctttctttttctctcttctgctagagtacaatttcgtgtccctcatactttaatgggaacattcaaattcacccagtaaatttgaattaaattcttagtctgctttccagattcctcagagattcattgatttaggaatatagtattcttaatatttaatagatagccagaaagagcacttctagcttgagcttattctttcatgttaccgcattttaaagagaacatacattttgttttgtgatttacagctgcagtttagaaaatactgaatacagaatgtagtatagaaacaattacattgtctctttttccattttcataccagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcaggtattaaaaggcaaggcagaaagctttcaatcacagccatgctgatttgagcaaataagactgaattattgaactttcctagcacagttctctttgtccaaaagatgttttaatgaatcctgatattaaattaaactcttcttatattcttattcagctttcgtgcaaagtagcactgtggtaggcttgaaggtgccttcatatctgttaattgatagcagccaatgtattactactaacatctgctcacctagactgcttgtgaatcaactttcacgtatctgagtcataaaaggagtggtttgtgtacatcagagggatcattctgctcactaaatattcattcgccttggtcatttgcttctgcagtataaaatgttattagagtgaactatgactgtagtctataataaatggatacttctgtaagtagtatatatatatataaaattagctctagttcagcaattcagacatacgtgcatgcatgtgcacacacactaagtcatttacaagtactttgaggttgtaggtccataaatttaagtagacagaagtcaactgtatgtgcttgtatgtaaaaataacgtgtgttcatttatgttgttttttttgtttgtactttgtttcagactgactgggtgggtgctgctaaaactgttcaacagcttcttttggaacattcaaattcacaaaggtcaacttgagatggttaaagctgcaactgaggcaagaatttctgtgaatctattaccccattttcccctcaattgtagagtggttttagggttcttgtggaaatgttttcttgtgtactgtgatgtatttcaacccatttggaagatttccctcatttatgataacttcaatagctgtgtatttggggaggagaaatgagatacaggagtacttactagcaatatgcaggattccttttgtaagttaccttgcaacaaccctgaaaatctggtagaacaatccctattttgtagatgaagcaatagagacacagatagataaataggttgtctcaagtggcagataataagtgacttgccctggatttcaaatcaaaccaagcctgactccaaaacctgtgctctttttatatcagtggagctgcttctcagaaagggtataggtcaactggaactcttgattgctcttcttcccccaacttatttctgttgttatagttgctctttttctttttagctcagaaagtattacattgaaggaagttcctattattggaaaatgtttttaaagagtgagtcactgattagccagtgtttgagtgactctagccaaatatgcagttgcaaagtgtgattgaattttacatttgagttttaaatttggcatttttccctttgctagacgaatttgccgcttctgtttctaccagttcatagatcccatattgactatctgctgctcactttcattctcttctgccataacatcaaagcaccatacattgcttcaggcaataatctcaacatcccaatcttcaggtaagatgagcttattttggaatttaaggaattaaaaagattatgttttgaattttaattttttaaattatagctttcgatatgttattgtaaagatgacattagccttttttcttttattataaaagtgatacatagttattgtgtaaaatttggagagtgcaaaaatgcattaagactatcatcattatttatatattttatatgggatacttggtgaactgtgactacctaaactttatgggcattgtttatcaaatagataaaaggcaattagaaaggcaagatagcaaattaccataccaggctcagcagtagggagttgtttggtggaggagggggcagtgaagtgggagaagggttagtgatataaagacaaccatacctgttactttgacctcataatttagtagagctagaaccacttaaaaagtacaatatcatgtgaaaggtatgttttagaggttttcaccttttgaaaggttttttagaggttttcctcaatgcgttccattcaggggaaatagtatgtgcaaatgcggagagatacaggagcatggtatggttgggaagtgatggacaattgggtaaggtgggaatatgggatgcattaggaaggatcagagactggaggtggtagctgtgtaggtagtgaaagctggatcaaaacaatttctaagccaagagattagagtctgtcctgtacttgagaaatgacatgttcaggtttgtattttgaaaggtcattctggattagaaagaatgaaatcaattctgcttccattaggatagttcaggcagaagataatggaggtttaaattgtagtattgtttttggacaggagggcatagatttaaaaagtacttagtagctcgaatttaaagacccagtgctagtccctatgggggctcaatagagggggagaggaaggagcaagattcccaggtgtttaccacctaggaaactgggtaggtggtaggtctgatcaccaacaaagaattggtgaggaactggttgagctggtttgggaaattaaaagtttcaaacctgaaaggacaaagcatctctaaggtcagctctgtgggtagagagtggataaaaggagcatgaatttagaaagagactggaaggaaagtgggctggcaggttgtgcaggtggagcttcacaagatggccttcgtcacctttgtgtgctgtgtcctcagtatcacagtctaaggagctccttcttaatcccttggttcagagttctataggctttcagagttctgtagctaaccagactcttgtgacccaactgaagagaagccatggattcatgcagtcagtgatatttatccagctgtgctttagacaagagagaacaaaatatatagtccttgccttcacatagcttaatggataatgtagacagacagctagctgtaatagcgtgtgatatgattctacagggtagatgtgcttttccaggaattagaatatctgtgtttgctctccaggggacctctctccaggaacaaggaaccgggaaactgaattagaaagtcatcaggaagtttctggtgggcacagacttagagggaagaggactattccactggggctctagagaagccttgcttctcttgtaattagaatatttcctatacccatttccttcacatttggttctcaattaggatttcttggaactgcttaataaacactgataattttttgtaatccaacttccctttcatggactctatgccagtgactgtgtagctcacctgtccttcagacacagcaggctgtagttagcccagcctgtttttgtgcagctttcttttgttacatgacagtctacccattcacaaaccacttctcaggtgttatttttgtggatggctcatctcattcattttattctgcagagttttactctcaaagagtgatactaagctttggcaacaccttactgtggcctccttctatttactgtagagcagtggtccccaacctttttggcatcagggaccagtttcatggaagacaatttccacagaccatgggggtggggaggatggttttgggatgattcaagcatgttacatttattgtgcactttatttctattattattactttgtaatatataaagaaataattatacaactcatcataatgtagaatcactagacggtcccaactgggggtgataggagacagtgacagatcatcaggcattagattctcataaggaatgcacaacctagatccctcatgggcagatgtgcagttcacaataggatttgcgctcctatcctatgagaatctaatgctgttgctgatctgacaggaggcggagctcagagggtaatgcaagtgatggggaatggctgtaaatatagatgaagctttgcttgcttgcctgccactcatctcctgctgtgtggcccggttcctaacaggccggggaccggtattggtccgtggccccggggtttgggaccactgctgtagaggataccaaaaatatatattaatgtttatgttagtatcactctacttgaaaagaataggaggctgtgagtttggaaagattgacttagacagtttcaacttcatatcaaaattagtgttgtagaaagagacatagggttaggaaaagaatcgttttatgcctgtgtatgtgcaaatgaagcacatttttgtttcatgttttggaaagatggttatgcttggtttcagttggataaaattgtttttgtctcacaaggatcctctgtaaacaataattatttgagaaaatttcccccaaggagctttcgtaataaacatcatattcattttgtagtaaacatgccatgcgcaaatttcagaatgtctaagggtcagtgtcttgagaccctgggcaggattttaaaatttagaagtgttctataatcctcttaaaatctatactatcatttttacgggtaatatgtcatgcaaactccttttggcttggaacagatataatagaggctcattatacacagtgatatcccaaagactgatgaacaatgtaaatgactgaaacatgtcgataatatggaaaatgaaaaaaatgcctgcatttttactgtcagctgctcaaacggcacatatggcttgtatccacttggcaaatgttttcaattaacttggaatgtgagtattctttttacatttttctttgtaaataattcatgtattctgaatttatagtaccttgatccataagcttgggggcttcttcatacgacgaaggctcgatgaaacaccagatggacggaaagatgttctctatagagctttgctccatggggtatgtgtgatctctaattatccttagggacattgtgttggttatcacagtggaaggaaaaatgactcttttgagttgagatttttaaaaactgctctttgagtaactggagtgattattgcttgagtttggtaatttgttgttgttgttgttgttgttattaccaaaatatagcattggaggagctgttttattagtgtgaataagatactttgtcagtagtgaagttatcgttttttctgttttgcttttaaagatttaatttttctgagctcattctaagttgagtggtcttgaatgtatgttttactttcttcttatatcccagcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactggtaagggtacctacacaaaagttttctttgcttatcaagtcttttagatttaacttttagaatttacatttgcttttagaattaagaattttttttggtactccctatgtttaaagcactgtatgaaatgtgattggatgggcatggtgagtagctttcatgccctatctttaacttgattggtagtgcttatgtggtgttttcttgagcttggcagaagtgacctggttagatttatcagtgccgaccttggtcatgggattgggaagtgggagaagacttgccaccgtgttgccatctctgctgtggaaggttatctgcggtttgttctccccagggcttaatgaatatgtgcatattttaaattgggcttgaagaacttgtggggtcatgttggcaaaaacacctacagattaatttgagaggtagcaatgactttttcagtatacctttttattgaagtataatagttacaaaagagtatataaatcataggcatgtagcttgaattatcacaaagtgaatacatctatgcaactagcacccagatcaaggaacaatattaccaggacctttgaaaccctccttgtgcttctcctatcatcatccccgcaagggtaatgattaatttcatttcttacagactgggttagttttgcatatttttgcacttacgtaaattggataacatgtggtctgtacgcatctggtttcctttgcttaagcaggtgtttgtgagattcatccacattgttgcatgtagtctattccttctcatagctgtgtagtattccagtgtatggtataccataatttattctactgttaacaaaatgtttggctgtaatcattcttggatattttttcttgatgaccatatatattcattttgttgggtatgtgcctaggagaagaactgatgggtcttaaaaaatatgtattttcagctttagtaggtactgccaaccactttatcagaatggttttaccagcttaccactccagtcagcagttagagttcaagaggaatttagttttgttcttatttattgtggaaattttggctgaggtacttatttcaagcttacggctccagacatgtcagaaattcaaggacgtctgacaatcagaggctttagaaattaagtgataggcttacaaattaagtgatatgatctgtatgacactcttctccttgtgttctatgaatctgtccttgatgtataccctactcatcctgactgtgtctcacgagttcatatctgagtctgtgtccgttatatgtcctgtcattgtgccttatgtgtaatagatactaggtatttactgaatgaatactcagctcaaaggccactgtaaggctggatttgcaattacttgcctaatattagacatttagactctggtgatcatcagacctcagataaatggcctctttgatgttttattcttgtttaagaaccacagaagttttccaggctatgtcattttttccttcattagtttgcaattgtatttttgtgaattgtgtgaacatgatactaaatgaaatagtattaaatgttttgtttttcattgactgaattccatctcccaatgacatgattacatctttgaatactaaacgttttctgacaggccgcagttaatgattacctcctttccttcttcattcattgttcttcttcaagatgaacttacagtttccataaaacaagttattctgtggataatagaatgtttgtgtatgtgttggctttatttctcaccatgttattctcactattggaatttagggcaaacctaagaagaatgagagcctgtggagtgtagcaagaggtgttattagaatgttacgaaaaaactatggttgtgtccgagtggattttgcacagccattttccttaaaggtgagtgtctgttgaaatgaaaccatgttgaagcatctacctggagtcaggaggctccagtgctgactcttaacagcacctctctgtgtgacttggggctgcagtttgctctcctgtaaaatgaggactatgataaaggtggtgttgcctaaatttttccacaaaatttgcctattgacaaaggagagtgagtatctgtttttgggagatatcaggctcgacatttattgctattattatttttttttaattcatgaccatctaaggggaacaaaaaacaggtagaaggaataagttttagtatttgatagtacagtagggaaattatagttagcagtaatttattgtatatttcaaaataactaaaagagaagaattgtaatattcccaaccccccacaaaaagctaagtgtttgaggtgatggttatcccaattaccctgattttatcattacacattgtatatagatatcaaaatatcacatgtaccccaaaatatgtacaagtattatatatcaattaaccccaaaagtcaaaaaaattttgagatggatgaatgaataaatgaataaataattgtaaaaaaataaaaattcatgggaatcttgcatgtgcctttataatacgtatttatatcttatgtttcaaaattgcctttaggggaaatttttccaaacacctcccctaaaagctgggtaactttttttttgagacggagtttcatgttcttgtagcccaggctggagtgcagtggcgaaatctcagctcactgcaacctctgccccctaggttcaagcaattctcctgcgtcagcctcctaagtagctgggattacaggtgcccgctaccacacctggctaatttttgtatttttagtagagacggggtttcaccatgttggccaggctggcctcaaacttctgacctcaggtgatctgcccgcctcctcatctgcctcccaaagtgctgggattacaggtgtgagccacttcgggcagccaaagctgggtgaatttgacaggataatgctctctatgtcctattgtgtagaatccagtacccacacatcccaaaggatagtgccatcctcattgaaagtctgggactggcaggtccctgaggcccttgctggtttaaattcttaaggtattcaaattgtaaaggagaaggagaaaaatgggtacagaagtagggagcccagggataaggagcagaaggtcccgcctctccttttattctacttcctggcttaactttaagagattccagctgctgtggtagatgcttgctaaccttaaatgctttgatcttttagaaccttatcatttcttgccactttctttctctttattttttaaatggtcacatagcttctctgtcttgttatgatttgcttttagattgaggttcttttgaaaataatgtgacccttgggttgccagatttttttaagtatgattatgtctatcatcactattaatgatattaagaatgtcagttttatgatgatgtgtgagaaaagttaaaaatttcttttaattttaaaatgattttgtttaagcatcctattctttgtgtcttcagtgttcaagttcagtttcatgcttagcagcattctgtgaatcattctggacttctgaagacagtttaggatatagaaagtatttcccatgctctatagcatggcttatttcataaacaactcccttttaacctttggaagatgaaattacattttatatgttggtgcgaaagtgagggtaaatgatcatgttgtgtttttttctccaggaatatttagaaagccaaagtcagaaaccggtgtctgctctactttccctggagcaagcgttgttaccagctatacttccttcaaggtaaaatttgtagaattgtttaatgctaatcaagctaccaaacatctgttatttcttgtattgcatagattaaccttattaaaaatgcgcacattcttattttcacttcttaagtaacttttctcttagtccaataagggatattgagatgacatgttttcatattagtttggaagtttttaattatttgcttataactttttgattttgtgactcatataggagttcttaattgatttatgctttttgaatgcttgaactgagtttttttggtagtaggttatgcatattgtatgtaagtgaaccatatgaaagaattttgctgtttgaatttttcggaattcaaattgaattcaaattgaattttaacttagagaaattgaatatgaagcctttcctttaatatgtggaagtgttgaattttcagaaatgttttaaaataaggcatcttgaattatgtattctttctgccagtggttttggaagtggtgtatcaacagactatttagtcataactaaataacatgttttggccaggccataatgtagtatattatggaggaaaataagccatagaaataatctgtttgtggtttaaatttaaaatatatagtaatttaaagtatctaattcatttaaagatcagttccacatggtttgccttgtaaaagagtgcggttgtgtgttgatttttgaaacactttttagacccagtgatgctgctgatgaaggtagagacacgtccattaatgagtccagaaatgcaacagatgaatccctacgaaggaggttgattgcaaatctggctgagcatattctattcagtaagtagaataccagactctttaattctacaatttagattcatcatggtatggtgcaaactatacagattttggaatctgctgaggttagtttccacttctattaattcacaggtggtgataggtaagttattagaaagaaggtgataccgtgaggatttgagataattggagttaaacatcttatacagtgtctggtaattagtaggtgttcatgtggtagttgttgtactttttaaaaatagttattattaaaatgatttgttgcctctgttgctcattaatgtgttttatttgaactacttgaactaatttgtggtacaacgtctagatttatagttgcatgtccatttctcctttgaccccatgagtttttgtaatgcagcaaatatgagtggccttgtataaagctttgccttagccgtggagaatacaaaatgaataattaatggttctgaactcaatgagcttatgttcttgtcagtgggatggttatacacaaataattaaagtgaacctagagaggagaggagtgtgagttcactgcaggaccagtcttctttaagttccaagctgtacaattgtgtactgttgtggaggctgctttaaaatcccaaggcagcacagtggttctttggtgctcagcctagttgtgatttctttcttgctgctctctgggccaggaccctgatgatactctccgcaccccctgccaatgctactggggaggtcttcctccctcaccttgctcctcccattctggaggcccctattcctgcttgactttgggtcagaggattctttctcaagcacatttgattatgtctctatcctgcttaaaatctggatacaaaaaataataccttattgttaaaaagtttgaaaataaagatgaaatagacagcaaaaacccattacccaaataaccagtggtgttgctgtgtagtctctaaaaatattttttacagttttgtattgtgcagctttccatttataacacaatattcatatttaaaaattctttataaatatttttaatagctatacatatagtactgagtacttttttttaacactggagacgacttcagttaccagtggtgaatgaagtattcttaaatttattgttatcttagaaaactcacaaatgttatcgtgatttaagattattccatgggtgttctcagatttgatttatataattataattttatgcaaattcagtgtaggggtgtgcactgtagtcattctcaaatgtgtgtctgtgcccttccttcttacctctgggtcaggttgaaagaggaaatacaaatatgctgagcacccattactgtggctcctgtgccgggccagtcattttattctcatgtggtctaatgagctaggtaggattttcattttatcagcaaggaaggtgaggccaggaatttaagtaatttgccccaagtcatgaagttagttagtgagtaaattaggatttgagcccagatttttctgacttcaagtcagctgttatccttctactacaccttcgttttaatatggtgaataccagcaagaagagtgaagtagagttgtagttataatagactgtaggtgggagcaagtaaaggaatatggaaatgggagatcccgtagaactaaaacagccttgtactttgggtcttgatatttcaataaaagtgtctcttcgtcattcagtaactaaattccactggcctggattgaagttggacctgtcttacttagcctgttgtatgatgaatagtgatctctaatgaggttccattataccagaacactgtagtattataacttccttttggtaaatcacacttatgggaaatgtgatagaagactttcacagttttaaatactcctgtttaaaatttaggtatttcaaaaaagttcagctatgtttgtaaaaagtgcaactaaaaaaagctttgtgtttgccttccagctgctagcaagtcctgtgccattatgtccacacacattgtggcttgcctgctcctctacagacacaggcaggtatgtctgcaatgtcacctgcagttaagtaggaagcaggagaatgttttgtcagctgctcagatacatactcaggggcagatttggcccactcatggctagtactatgaggtaaacagtaaaataagctgaggaccctgcccaaaggagcttatcaacttggtggaggaagaatctacatttaaaaacaaaacaaaacagactgcttaatatatatagtactaagctattgcttgattaatactgttaatgtaaataaggtctaactcattaacatgtggtatatattctgaaagcaaatgctttccaaatgaattgactggcattttagaacccaccctttatcctcccaaaatacatttgtctgagctttatgatggaagagaccacaccagtcttgttcagtcatattctcaacaccaaacagtgccaatatatataggcaccaaataaatatttgccagataaatatttaaaatgaaggaaattttgctaaaaaataagcactccttacttgcagatggcttttttcctcccccttgtgatcttatgtgtgcatatcatgtgtcatggctagagtgttcactagcatgtgtaataccttgacattcctggaggaccagctttttaagtagttttctgactcgctctgcattggtgttctggccctacttctctatgtgctagcagtgagctgcaggaaagccaaggcaagaagaaagtcttggaatgttggcctctcagctttttggaaagtaggttttccaccatcatgataccgtttccatctttctttagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcataggtatgtcagaccttgaaatattttcagtaattttcatggaaaataaaaaggcctaaaacacatagtggtatctgctttaatttgggtgatagccccttgcttgagggaatgtggctataattttagttttcaaacgaaaatgtaatctcctgttatatcactgtttacaatgagtagttactgaattaccgtttgtatagcattacccttatttttcaggagaggtagagtgtcttgctatccaggagctgaaaatctaactgagatgagacatacacagagcagcaaagtgagtggagggcctcatttgtgattaagagaaaagagaggaaggagagaagagggggcttatgtctttggagaaggtttttgatgcagttgggatcatatggaatcataagaatttagtcttatgggatttacttaaatttctccaaatacaggccattatttttgatgtattaataatctcttaaggttattcaagccagttgaagtttttcttggagactttgagctggcaacttcattgttgcttttcttttcttagcttgcagcctttatgcagttctgaacaagaggggactggggggtcccactagcaccccacctaacctgatcagccaggagcagctggtgcggaaggcggccagcctgtgctaccttctctccaatgaaggcaccatctcactggtgagtgaagacccttcagtgttgcctggtctcttttcttgagcgttttcatgaaaacgtaaccccccgtgtttttctgttagataatgtttgacccattgttgatactttatattccactcctggatttcagtgaccaccaaaccaatatgtcatttactatcttggtttgctctctgggtcactgctccactgaatctatcctgcagctatgagccatgataggacagattgctcaatgttctcaggcagtcacatccaaagctagtgagcacgtattgtgtgtgaggcactgggtcagtggtttgtaaataattaacacacccacctgtagatgatggaaccacagatggtaagagtcagaagcagccttaaaaatcatctgctcggccaggtgcggtggctcacgcctgtaatctcagcactttgggaggctgaggcgggcagatcacgaggtcaggagatcgagaccatcctggctaacgtggagaaaccccgtctctactaaaaatacaaaaaaaaattagccaggtgtagtagcacctgcctgtagtcccagctactttggaggctgaggcaggaggatcacttgaacctgggaggcggaggatgcagtgagctgagattgcgccactgttctccagcctgagtgacagagcgagactccatctcaaaaaaaaaaaaaaaaaaaaaagaaaatcatctgctcaacacctctcaatttacaaatgaggaaaaccaagcccagcagtactaatgagcatttatattatatacccaaacacttttaggcatttatcatgagacatatgtattaagtagtagatttccttcttctcaaagtcatcaacatgtagaattcatattttatttgaattccgatattttcttttgtatgaatgtgtcttgagatgttgacgagatgagtgcatgtgttaggagtaccgccccctccatgccatgctttgtgtgaggtgtgctcgtggtcctgttggggaatctgttttgcatgaaccttgggtggtgggatggaagggatgagggcagataagacttccaagattgaccttgtcctttctctggtatttatttacttattccaatgttgcctttcctcaatcaccacttttgtgtacagtatttattataagataactattgcagagaaattgtcttataaagtaaaagaaatagatggtggggggctgataagcagtcttcagagggctttccacactgtgggtgtggggagagaattgagctcaggagtaccccttgcaggtggtgcagtgttgggtgctctgccaactttaagtgacagaggtcttttcttgggctttgtccatgaccgtgtgtgtatcaagacagtgggcactgggaatcacaggacttgctcagtggggtgatggaggtaacagaatggaaggctttggcagctaccttgtaccatgttttctgcgtttaaaaaaagaagtataaaatggtggcatttatatttaaatgaattttcttttcagccttgccagacattttaccaagtctgccatgaaacagtaggaaagtttatccagtatggcattcttacagtggcagaggtaagaggtagagctttcctttatttgtctttatacttactcttcattcccttaccttttctacaacttatttaaaccaataattggccaggcatggtggcatgtgcctgtaatcccagcactttgggaggtgaaggcgggtggatcacctgaggtcaggagtacgagagcagcctggccaacatggcacaaccctgtctttcctaaaaatacaaaaattagctgggcatggtggcacacacctgtaatcccagctacttgggaggctgaggcacaagaatcggagtggtggaggtttcagtgagccaagatcatgtcagtgcactccagcctgggtgacagagtgagactcagtctcataaataagtaaacaaaccaataattacatttggtatatagtcatgttcagatgtagagaagcaaaaaataagaatggggagaataagtgtcctttgggattatttaatattttggttcctatttgtaagttgtgttgtctaaaacatatcctgacttgattccgtgacatccagaagtttgtatgctaagttgagacgttacacctatcagtatgcttgcagcttgcacatctgcagttttcatttttgagattctattttaattttggtagatttagtgtagaggagatggcctggaaaagtacatatcaatttagaacatgtgggggtacttttatttgatctttctttctggtagctctcccctcccacctgccctttcttttgtctcctgctgtctcctgactgagtttgagtttggaagggaagtggactgacattatgagtggctaaacagagaagagccaggaagggtcagagaagaggactgccttgtcctggctccactgggcagttgtttgtgtgccagtgcatatttatttactatccatcatctccttggcctagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtacttgggtgaagaattctggtggatattacagggatatgttgaggtttatgctgcagtgagatcagctggagtcctggtgatgtcttcttatctaaagaatcccccaactagctctggtaccttctgtgtggtaaagacactcaaactgtttgagttgaattaatagcattttaagtagaaaaggaaaggagagtctgaaaagtcaggaagatgaatgtcataggtgagactttcaccatccttttatgaaatacacaggtgcatacctgtttacctacacctgcacccctcatgaggcagcagttttgctattgagctgccactgacctggctgctctttttgagtcactcttgctgtccctcccaaaatttcatatattaagctctttgctgtcaattaaaacaaataccattataggagaaaattgagattaaaaaaaaagtccctgatttagaaaaatcaattttgtctaatttataattttagaacttagtaataatgacccgtcttttctgaatactctaagaggattactcttttttgacatttagaaattgtcttctttttcacttgggtggtattagtttagtttcaagatggggcagtgatcttgctttcacactccagaggggcttgaccgaaccagtgtgttttgggtaggtacagtgagagcctctcggccataaagcaccgccgtcacagtggccatcattccccacagtgctggactgtgggcaaaggccatttaggggaggcagggaataggtgctgcagaaggagtgagataatcttggtggtctcccattggtctttctgcagacttgagtgactgttgagcccaggcttcaaaccatggagggcctggtcttaggagcggctatttttagtgatgacagcgtatcacaagtagggcattcatttatgaaaatttcttctaggtggctgttcaatgaacacaagcctcaaataccataaaaaagtgaatgattacaataaagaatgtgtttgaaagccagcagttgtttccagcagagattctctgcatgaggggcagggggccgctttcatgtagtgctgatgtgagtggccatcttctcacgttactggctctccagagaaagtcctctgtccacttgccttgtgtctcttgtcccttcctcatgacttcatctcctgcttttgcacactcaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatggtatgttaagtcactatttattcttttaaaatcttttttttttttttggatttcagaaatttgctaattgtagaaaattggaaaaatgcaaacttatcctgacctctaacaccgtagagtattactattatcttctttggcatgttatataagttgaatgatacagttatacactcttacatctggcttttttcacgtaacgttatttttgagattcatacatgttgcatatagttgttcctttgttcttgttgtgtagtgttccattgtataaatatttaccacattttattcattctgctgttgatggatatttgggttgttttcagttcacagctgctttgaacagtgttgctatgagcatatttgaatatgtcttttggtgaatatctgtgcccctatatacagcatgtatgtgggaacagaattgcctggttgtaggctctgggtatattcggctttggtagataccaccaaacagtttccaagggattatacctacttgtactcccacaacagtgtgtgtttcacttgctgcacatcttggccagcgctccgtcttctttgttttaggaggtgtgattggggtgggagtactattttatacagttgtagtttggagctgaaactacttgaatcactttttagagccaaaaggaatctaatctaatcacctcattttatagttgaggaaaataccccaggtcacactgctaattaatgggtaatctcgggatcagaattcttgtttcctaacctcgaggcctgtgctttctctggttccactgatgtacaatcatctgtgtaccactgggtagcttaaagaatatttatacactatctcatttgattctcacaacagtcctggaaaatggatgttttaggtatttctacttccctctgttctccctatttctctctctacaccctcttttctttccccttccctttcttatttacctcaatataggacaaagtaggtgtaagcaaagtaggtttaacagcaagtcatctgagaactcacaggttgccagtggtggaagtggggccccacccttgtctgctgacttgccctcattctcttcctagctccccatactttcttactgtggctgctgggattgtcatgatttgttgagcgtaaccatttgacagggttttattctctctcttcagctgagagtgccacatattgtcttgtgaagaatgctgtgaaaatgtttaaggatattggggtaggtgtccaccatttatggtataaaagctatctcaacttctgttctctttagatctagtctgtttgagctacctttgtggtggggtggaccccgagagaagcagtttctgctggcttaatgataaaggcatttttgggaacggggcatgtaggatgggttgggctatgttgaaggtgaagcccatcagtgtagatttatttgaatgttctggaattttactggtttcacatttattcccaagctgctatatataactggtatcaatatgtttcaaggtgctacaggttgaatatcccttatccaaaatgttgggaccagaaatgttttggatttcagatttttttgggattttggaatatgtgcattagacttacaggttgagcgtctctaatccaaaacccaaaatgctctgatgagcatgtcttttgagcatcatattgatgttcaaaatctttcagattttggagtacttcagatttttgattttgggattaggaccacaatcttggctcttaattattccatatgatttttagttactttcttgtatttttacaagaatttctagaagtatcctcaggtgtcctttaccttcatgattcatggggaatgtaagttctatagagatactagcggccttttgtgagggactgtttgtgactttattctaagtcattttagagtatagttatgttggttgaaattttagaatatagttaatgttggttgaactccatcaaaacaaaaaaaaaaaaatagaaaaagaaaaccttgtttgcattggaaactcccacctcatgctcacacacacccaggcatacatttacttaatgccaggatatggttttttgtgcctttactgccttctgaggtctaggacaccacaggtcaggttggtaatggaaattttttttattttttattttttgcctttaatttaatctaaaccttttctttaggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacata

SEQ ID NO: 2 - DNA, 33901 bp, Homo sapiens
  source 1..33901: mol_type = genomic DNA
gcgccactgcagctggcattggccgggactggaagtgcgggcttctgcagcagccgaagctggagctgctagggtgcgaactgccagggcaggtgtgggctgcgggaggctactcgggccgggagcgccgggacaagtgcatggaggtggtgcgactcctgcaagttgctggggcagtcgtgtcccctattccctgacacccgagctccggtcttctccctgagaaatcggtcgggctcgtgcaggggtggggatcaggattcatctctcctggggagactgcgctggggcatgggttcccctgggttggaaggagtccttatggactcagtcccctaagtatctgcacgcgcggaggcgccgccagccagcccgcctgggtccctagggaacattgaggcagtaaggggtactctgagagcagccggtatgctggagataattaatgacagcgttaccattgattggactgagcgctcactgtgtgttacacactgtggtagaccctttgtttatattattccgtcctctcaacaatccaggaggttaagagctatcattctgcccattttccagatgggaaaactaaggcatagaagttaaccaacttatggtaataaaaaatcacacagatggtaagcagtggaagatttaaacctcacagctggtcttccagcctctgtccttttcagatcctctagtcagcttctctcagcgcatgctgctggctggtgcctctgggatattgcttctttattactccttgacctagagcgaactgtgcggagggaggaagtgcaaaacgtggacctcctgttggcacaaacgagcctctttgtcctctcctcatcctctcttcacttcattttctcaggcagcagcggctcccctgttgtatggacattctgcacccgaaactgatagctgagtcctgaagttttatgttatgaaacagaagaactttcatcccaggtgcattggtttattctctcccctagcctgttatgttagattgtacttatgttaaatttttaaggacataggtggttatgccgagaataacagtgactgtagccaacccatgtggggattgtaaagaaattggaatatgccttccaaaggaacactgttaatctagacatgacctttcaggattttaaaggaataattttaggccaggtgagaaacagccaaaagcaaaaataatgtgaggctagagattatgtttgaaagtttttatattttggattaatatctattctaggagtttactcttagtgaatctttttccttagggtagaaaaaagcttatcgaaaaggatgaaaacttatacatcagatatttcaaaatagtgtgcttttctttcagagctttgtagctttgtattacctttgtattaaaagtgaaaatgttcttttaggacatcacaagaaaattaagatcttaatttaagtacgggaacttaaggggtgccctaattgccttaattaaataaacatgccttccttctgccaggaactattcaattaacaaaaatgaaataagaataagctcttattttcatttgcaaatgactattcatagctattttgtttggatacaattcatagctattttgtttggatactattttgtttgcatactatgttttgtttggatactattcatagctattttgtgtggatacaaaggatacaatgttgtatccttatatattcaggaagtcttagaatacaaatgctgattttttttttttctagtcagggaagcctaactgtgttcatataaacacatcaaagtcagtttttaccttgtgactcaacatggtagaagtctgtcagttcatgtgatatcccattgtggagcaaagtaatgtagaaatcagaattttgagttctagtatttactctctcgattccttgttaatttaaatggtacctattttttatagcacatgatttgggaattacactttgtgacatggatgaatctgcactgacccttggtacaatagatgtttcttatctgccacattcatcagaatacagtgttggtcgatgtaagcacacaagtgaggaatgggtaagtttaggataagagttaaaagcactcagcctaaaaatgttcatttcaatgatgtttatgaaggtgcaaatactggcagcaacataatatcacatcaagattaatagctacagaaactggtatgtttatgaagcctaataaaaaattttatggcattatattgggaagaaaaaatggttagaaaatttatgtacactgattatgttagaaaacaaaataatcattttgcataggaaaaaaaccctagtagtagttttgggtcggtaagaaatttttttttctttctttctcttggtatgtactttccatgagttatacttgaaaagcatgaattttataatagaagtaaactttaaaaaattaggaacttatagtatgaattcaatccaaaaggcgtacattttcttggattgtttttcctatttcaatataacatgacgtattttaaatatcttaattgtctttatataaaaaatggtggggtttttttttttgcttaattttgagaatgttatatctgtataacagagatgtgagcaactgagctcaaagttaagcatttttagaatagaagagcaaggtttgaccctaattgaatagtatagctataatttaagttctggtctttaagttcccttgataagataaaggaatgttatataagtttacctgttttttatttaaagtaaaattataaaaacttgctcactatgtaaggcctttatagttctgtagtgcgactgtgtgatttaaaagggggtaaggttaattgttagactagagatccaggagtctgaattgatttatctagctgtcagaagtctgtttgaccttctacaatttagtttttaaatttttgaatactgccttcagtgtgtagctattactatccttgggtgctcagcttttgcttatgtggtaagcttttatccaagtgttgatcatggagggagtacttaaatgaaaatcacccagaatatgccataagtttttcttctgttcaaaatactttagattttgagatcaagtagacaagtattctcttgccatttagaaactatatttttttcttaatatttgtcagggtgagtgtggctttagacccaccgtcttcagatctgcaactttaaaatggaaagaaagcctaatgagtcggaaaaggccatttgttggaagatgttgttactcctgcactccccagagctgggtaagaaccttactgcttattgaccctaatactcagcataaaatattatttccaactttgtagtaggcagtcttattggactaaattcactttcagaacagagacccatgtttatgaaccaaatatttcaagtaaagatgttgacacactttttctttccatttatttattcagtaaatattggtcccataattgatactaggtaacattctagttcttttgaggttttagtaggattttaagggggagagtagatcttacttttggttaagtcttcaaaggagttagtgggaaccttcatgaacttttaggtgttgaactttagcacttaacattaaaattctagcaagataagaaagagataatgggacagaataagaagttaaagctgtgactttgctctgagcctttgctaatggaaggtgtctgttgactttgtgatagtgaaagattgtcactaaaatgttagaatgaagatttaagagagtgtatatgggtttgttgtttgtattttggtgaaaaaacacaagccatgcaatatcttttgcattacagtgcagtctttgagaagagccaagtgggtgagaggtatattttcggtggtagttgaagagaaggacaaattagcacaggaacaagaacttcacgtagttgtgtttgaaggcagtagaattgccttttaaaagtcatatctggatgttaagctctctctgggatccagttattaggatgaagaaattctgccgtttaagtgcctgccatttatagaggttgcttgtaacttgtgtggctaggtaattgtgctgtgtgaattttctactcaaggttggtttggcagaaagtagaattctgagtctggtataaaggggtttactaacatgggagagatttgtgtggaacccaagcagtttatgttaaatgttcagatctgctgaagaatgtcttgttataaatattgcttcttttaaaccacaactaccttgttgcaagaatttggtagaattcaagaatcacagaaatttctagcctaggagtaaagctagttcagattaataggtcacttgaaatcaagatttaaacagaatgtcattttctttttagatctgacatctagtctaattctaattttgaatacttaaaacaataactacaatgtgttcttaagtcagagggtataggaaataatttggtggtggtaatggtttggagtaaagggtaaaggtagatagtacaacagacaattgatttttgccctactatagtacctgatggagttgaacttgatcttgaattacttccatgacatgatcatttaaaataatttgtttaaaaatgtttttattggtaactgtataatatttgctatcttttctctgaaatcgtattttccttttctctaaagagattccacttaagtagttttatatttgttgagggaacattgtgctttttcagcatttaagtgtggaacagttacctttttgcccatattgataaagctatagactatcttttcagcaaagctatctctggattatctttcaagatcacatgtaaagagggtgaccagtagacagacgatttctcattaaatggacagttatttaggggtaatagaaagaccatactctttagatcaagacataagattgaatcctaaacctctcccggtcttagtttctcacctagaaaatggtgataactcttttgtgtcagggattttgggagatttagtaagcaacaataactgcatggtacttagcgtggtctttgtattttgggaaaactgaaatctctgacattcttgtagcttcacttctttcagggtggagatgaggagtcaagcagcaaggggtactgtttcctttggttttaaggtggtttgtttgtttgtttgttttttgtttttgtttttgtttttttgagatggagtcttgctttgtcgcccaggctggagtacaatggcatcgtgttggctcactgcaacctccgtctcttaggttcaagcaattctcctgcctcagcctcccaagtagctgggattacaggcaccgaccaccacacccagctaatttttatatttttggtagagacggagtttcaccatgttggccaggctggtctcaaactaatgacctcaggtgatctgcccgcttcggcctcccaaagtgctgagattacgggtgtgagccactgcgcctggctagttttaagattttaaagatgaattttgagtttatgacattctttgaaggctaaaaagtacttttcaataaaacttttttcttccctaagatttttgaggcaatatctgtttttatgcttgatattaaagttatcaagaactcataatgagagctcttcaccgtcaatctagttgtgtatttatgtctacatttactttgttttatataggacaaatttttcaaccccagtatcccgtctttgggtttgcggaatgttatttatatcaatgaaactcacacaaggtaaaaagaaatttcttatgtttttaatacatctcaaaagaaaaacttcataataatcagttaacacactttaagctgaattgctttgcattactgagagtgaatagaatatgagtttctgacacaagagagattatctgaaaattggaagcagtatagccacagagagaactattactgttaccacttgtgtacttacagataatttcaggggagtgtagatgtcacttagaatgttctttgacttatattgatttgcatatgaagtatgccaaaatgtgggaaatatttgtctgagaagaccggtaggtttgaatctcttatgatttattatgactatttaatacttaaaaaaagtataaattattgaaatctgcccaatctgtagggtgttaaaagacattttctctgttgagcaaatgttgtttgagttactatttacccaagactttacttagagttcttacaatttctaggcatatggggataggcttgtaagcaccagtgtgagagaaaaaggtctttgttttatctgactctaggccaggggacagggaaggagggtcgaaaggttgtgtgaatgtctgtgaggcaagagaatcagtagccaaatgctagcagtccttgtaattctaaaaatagatacctggagtaggcaggcagataggctcagatgagaaatacttgaaattctatgaaaattagtgcaagaaatcaactacttgaagcccttatgatattacttctcataatttgatgatgagatcacatatgagaatgataatacttggatatatagtaaacttttaaaaatagaacttaagtctaagtacatgagcagatcattcccaggctgctggtctctctctctctctcttttaaatttgcatttaaatttaaatttaaaaaagaggggagagactttggtccgttgaattttttttgataagatgataagatcctaagaaaagggatctcagagtcttccttatgtaagtagatattttatatataaagccaagaggtttgtttcttgccctcaattagagacagttaaggaaaatcgcatttgctatgatgacgaggcagaatgaaactattcagtgaaataagctgcatgttcagctatttacttcttctgtgtattctatgaagcaaagcctaaaacatttttacacattcacatagagtcacacactctgtcactgtcacactcatgcacatgtgcgcacacacacacaatgctttcagtcagttgcaaagggatttcatagagagcttggactttgtacataccaataatatttctgaaaatgttgactgaaggaggagtttaggattaacatttaatatattgggccaaatttttggaagatttagacaaaggaagggaggaagaaaaagagaatggcaggaaggagaggtgaagaaataggtgattcataaacagttctctttcaagttactttttctctattgagggtattctgggtcttttcagagttactgaattgataataataatgtgatatcatgtaatcattacaaatgttttcaaagtattacataatgaaagcttgtagcattcttattctgaaactcatctctccatcctcccatctaaacaccacatagtcatttataactgacaggaatatgttctatccctcaggtgtatttaaaatgtgaagggggaaaaagtgagaaattctggcttcgttagcctagagtacaaatatttagatcatgcttgacttctttcaactgatctgccttgtcaattcaattagagaatcagtgattatcttaggaaaaaacaaacaaaaacttaacaaaaaaaattagtctttgatcataggaaaagacttccaaatttatctttttatagcccatagctataaatataagctgtatggtttatcagaatagatatagcttatatatagcattaaacatttttgcctttttaatggtattaataaagccagatgttcttatttcttttgttttcatattagggttattcggcacagtaacactattcaaacattaatacacaaattccaagtttcagttttagctgagagtacatgaataaaattggctgcacaacattctgaactactaaggagaatttgctgtaggagaaccctaaatctcagaaagagatgacattaggctttgtttttatgggcggaaaattagtttatgatttaagcaacttgttttaggggtacctctaatacttctctgctaaatcattttcacagacaccgcggatggcttgcaagacgcctttcttacgttctttttattcaagagcgagatgtgcataagggcatgtttgccaccaatgtgactgaaaatgtgctgaacagcagtaggtaagggcggggcaaggtgtaaaggtgggaagaggcagggatgggcggtaggagtgggagggggaagtaattcttagtaacctttagtttctctgggactgtgtctcaagaagtgttcacatttccaaacattaaatgttaagctccaaaggaaactaaagaaaggcaggagaacaggtgctttggtgacagtgccatgtgggaatagcagccttgttttatggattaataattgaatgggtttctaaaacatgaaaggcaaataagagttcagagatgcacatatttccagagttctctgctttctgtaatataaatgttggagacattgtatttcctattttatagccgaaagagaattgagtttgatatttttgccagttactgagtatctaggtacaaattcttgtactggaaagtcattgctagaaatcttaacagaatgaattactgcctttgctcttacaggagcttgcttacaataggaaagactgattggaactcaggtggaatggggaaataaatattctagaatatgggagtttgggtgaagatgttccaaactcccgtactctagaatattgattctagagtactggcctcattggtgtggccagtttcagagatctacctggatttggggagcaggagaccagggtgaattaaaaggctgctttaaattcataagtgtcatctgtagactcttcgttatgtttgttaaggcctgaacaatgctggcacataatatgtactcaagaaatacttctcgaatggatgagtgagtagttgataatggaggtgcatcagtcatggtaatgacgtgtagaaacattcctggtcagaatgcaatgaatgctttttagggaatagacttggggggcatcagtgtcctgtggtattggtctttttgtattagtgaattatggggctatgtggaattttgctgtacttcactagtccaaaatcagcaccactgggataaaacacaattatgtgaaattcttagattattatatattctgttttcttgttaaaaccttcaagtacatttatttcatccagatgttttgactagtaactgggatgttagaagtggtttaaggaggttgaacctcatgctttctcccctaagtcagctggaagatttataaaactggattaaatgtttccttttatctcactgcttattattctgtgctgtgctcttttgatcttgagctgtgccttttaaaaattactggtttgagttgaaaaaatttgtaaatatttatgaccatttttggtccctcccagagttttacatatagttttactaatggagtcacaatacaaattaaaagtaaattaagtcttcaaaagaaaaatcacacattaatttggtagtagacttttggatacctctgaattatatgcctaaagggataacctttctttttctctcttctgctagagtacaatttcgtgtccctcatactttaatgggaacattcaaattcacccagtaaatttgaattaaattcttagtctgctttccagattcctcagagattcattgatttaggaatatagtattcttaatatttaatagatagccagaaagagcacttctagcttgagcttattctttcatgttaccgcattttaaagagaacatacattttgttttgtgatttacagctgcagtttagaaaatactgaatacagaatgtagtatagaaacaattacattgtctctttttccattttcataccagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcaggtattaaaaggcaaggcagaaagctttcaatcacagccatgctgatttgagcaaataagactgaattattgaactttcctagcacagttctctttgtccaaaagatgttttaatgaatcctgatattaaattaaactcttcttatattcttattcagctttcgtgcaaagtagcactgtggtaggcttgaaggtgccttcatatctgttaattgatagcagccaatgtattactactaacatctgctcacctagactgcttgtgaatcaactttcacgtatctgagtcataaaaggagtggtttgtgtacatcagagggatcattctgctcactaaatattcattcgccttggtcatttgcttctgcagtataaaatgttattagagtgaactatgactgtagtctataataaatggatacttctgtaagtagtatatatatatataaaattagctctagttcagcaattcagacatacgtgcatgcatgtgcacacacactaagtcatttacaagtactttgaggttgtaggtccataaatttaagtagacagaagtcaactgtatgtgcttgtatgtaaaaataacgtgtgttcatttatgttgttttttttgtttgtactttgtttcagactgactgggtgggtgctgctaaaactgttcaacagcttcttttggaacattcaaattcacaaaggtcaacttgagatggttaaagctgcaactgaggcaagaatttctgtgaatctattaccccattttcccctcaattgtagagtggttttagggttcttgtggaaatgttttcttgtgtactgtgatgtatttcaacccatttggaagatttccctcatttatgataacttcaatagctgtgtatttggggaggagaaatgagatacaggagtacttactagcaatatgcaggattccttttgtaagttaccttgcaacaaccctgaaaatctggtagaacaatccctattttgtagatgaagcaatagagacacagatagataaataggttgtctcaagtggcagataataagtgacttgccctggatttcaaatcaaaccaagcctgactccaaaacctgtgctctttttatatcagtggagctgcttctcagaaagggtataggtcaactggaactcttgattgctcttcttcccccaacttatttctgttgttatagttgctctttttctttttagctcagaaagtattacattgaaggaagttcctattattggaaaatgtttttaaagagtgagtcactgattagccagtgtttgagtgactctagccaaatatgcagttgcaaagtgtgattgaattttacatttgagttttaaatttggcatttttccctttgctagacgaatttgccgcttctgtttctaccagttcatagatcccatattgactatctgctgctcactttcattctcttctgccataacatcaaagcaccatacattgcttcaggcaataatctcaacatcccaatcttcaggtaagatgagcttattttggaatttaaggaattaaaaagattatgttttgaattttaattttttaaattatagctttcgatatgttattgtaaagatgacattagccttttttcttttattataaaagtgatacatagttattgtgtaaaatttggagagtgcaaaaatgcattaagactatcatcattatttatatattttatatgggatacttggtgaactgtgactacctaaactttatgggcattgtttatcaaatagataaaaggcaattagaaaggcaagatagcaaattaccataccaggctcagcagtagggagttgtttggtggaggagggggcagtgaagtgggagaagggttagtgatataaagacaaccatacctgttactttgacctcataatttagtagagctagaaccacttaaaaagtacaatatcatgtgaaaggtatgttttagaggttttcaccttttgaaaggttttttagaggttttcctcaatgcgttccattcaggggaaatagtatgtgcaaatgcggagagatacaggagcatggtatggttgggaagtgatggacaattgggtaaggtgggaatatgggatgcattaggaaggatcagagactggaggtggtagctgtgtaggtagtgaaagctggatcaaaacaatttctaagccaagagattagagtctgtcctgtacttgagaaatgacatgttcaggtttgtattttgaaaggtcattctggattagaaagaatgaaatcaattctgcttccattaggatagttcaggcagaagataatggaggtttaaattgtagtattgtttttggacaggagggcatagatttaaaaagtacttagtagctcgaatttaaagacccagtgctagtccctatgggggctcaatagagggggagaggaaggagcaagattcccaggtgtttaccacctaggaaactgggtaggtggtaggtctgatcaccaacaaagaattggtgaggaactggttgagctggtttgggaaattaaaagtttcaaacctgaaaggacaaagcatctctaaggtcagctctgtgggtagagagtggataaaaggagcatgaatttagaaagagactggaaggaaagtgggctggcaggttgtgcaggtggagcttcacaagatggccttcgtcacctttgtgtgctgtgtcctcagtatcacagtctaaggagctccttcttaatcccttggttcagagttctataggctttcagagttctgtagctaaccagactcttgtgacccaactgaagagaagccatggattcatgcagtcagtgatatttatccagctgtgctttagacaagagagaacaaaatatatagtccttgccttcacatagcttaatggataatgtagacagacagctagctgtaatagcgtgtgatatgattctacagggtagatgtgcttttccaggaattagaatatctgtgtttgctctccaggggacctctctccaggaacaaggaaccgggaaactgaattagaaagtcatcaggaagtttctggtgggcacagacttagagggaagaggactattccactggggctctagagaagccttgcttctcttgtaattagaatatttcctatacccatttccttcacatttggttctcaattaggatttcttggaactgcttaataaacactgataattttttgtaatccaacttccctttcatggactctatgccagtgactgtgtagctcacctgtccttcagacacagcaggctgtagttagcccagcctgtttttgtgcagctttcttttgttacatgacagtctacccattcacaaaccacttctcaggtgttatttttgtggatggctcatctcattcattttattctgcagagttttactctcaaagagtgatactaagctttggcaacaccttactgtggcctccttctatttactgtagagcagtggtccccaacctttttggcatcagggaccagtttcatggaagacaatttccacagaccatgggggtggggaggatggttttgggatgattcaagcatgttacatttattgtgcactttatttctattattattactttgtaatatataaagaaataattatacaactcatcataatgtagaatcactagacggtcccaactgggggtgataggagacagtgacagatcatcaggcattagattctcataaggaatgcacaacctagatccctcatgggcagatgtgcagttcacaataggatttgcgctcctatcctatgagaatctaatgctgttgctgatctgacaggaggcggagctcagagggtaatgcaagtgatggggaatggctgtaaatatagatgaagctttgcttgcttgcctgccactcatctcctgctgtgtggcccggttcctaacaggccggggaccggtattggtccgtggccccggggtttgggaccactgctgtagaggataccaaaaatatatattaatgtttatgttagtatcactctacttgaaaagaataggaggctgtgagtttggaaagattgacttagacagtttcaacttcatatcaaaattagtgttgtagaaagagacatagggttaggaaaagaatcgttttatgcctgtgtatgtgcaaatgaagcacatttttgtttcatgttttggaaagatggttatgcttggtttcagttggataaaattgtttttgtctcacaaggatcctctgtaaacaataattatttgagaaaatttcccccaaggagctttcgtaataaacatcatattcattttgtagtaaacatgccatgcgcaaatttcagaatgtctaagggtcagtgtcttgagaccctgggcaggattttaaaatttagaagtgttctataatcctcttaaaatctatactatcatttttacgggtaatatgtcatgcaaactccttttggcttggaacagatataatagaggctcattatacacagtgatatcccaaagactgatgaacaatgtaaatgactgaaacatgtcgataatatggaaaatgaaaaaaatgcctgcatttttactgtcagctgctcaaacggcacatatggcttgtatccacttggcaaatgttttcaattaacttggaatgtgagtattctttttacatttttctttgtaaataattcatgtattctgaatttatagtaccttgatccataagcttgggggcttcttcatacgacgaaggctcgatgaaacaccagatggacggaaagatgttctctatagagctttgctccatggggtatgtgtgatctctaattatccttagggacattgtgttggttatcacagtggaaggaaaaatgactcttttgagttgagatttttaaaaactgctctttgagtaactggagtgattattgcttgagtttggtaatttgttgttgttgttgttgttgttattaccaaaatatagcattggaggagctgttttattagtgtgaataagatactttgtcagtagtgaagttatcgttttttctgttttgcttttaaagatttaatttttctgagctcattctaagttgagtggtcttgaatgtatgttttactttcttcttatatcccagcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactggtaagggtacctacacaaaagttttctttgcttatcaagtcttttagatttaacttttagaatttacatttgcttttagaattaagaattttttttggtactccctatgtttaaagcactgtatgaaatgtgattggatgggcatggtgagtagctttcatgccctatctttaacttgattggtagtgcttatgtggtgttttcttgagcttggcagaagtgacctggttagatttatcagtgccgaccttggtcatgggattgggaagtgggagaagacttgccaccgtgttgccatctctgctgtggaaggttatctgcggtttgttctccccagggcttaatgaatatgtgcatattttaaattgggcttgaagaacttgtggggtcatgttggcaaaaacacctacagattaatttgagaggtagcaatgactttttcagtatacctttttattgaagtataatagttacaaaagagtatataaatcataggcatgtagcttgaattatcacaaagtgaatacatctatgcaactagcacccagatcaaggaacaatattaccaggacctttgaaaccctccttgtgcttctcctatcatcatccccgcaagggtaatgattaatttcatttcttacagactgggttagttttgcatatttttgcacttacgtaaattggataacatgtggtctgtacgcatctggtttcctttgcttaagcaggtgtttgtgagattcatccacattgttgcatgtagtctattccttctcatagctgtgtagtattccagtgtatggtataccataatttattctactgttaacaaaatgtttggctgtaatcattcttggatattttttcttgatgaccatatatattcattttgttgggtatgtgcctaggagaagaactgatgggtcttaaaaaatatgtattttcagctttagtaggtactgccaaccactttatcagaatggttttaccagcttaccactccagtcagcagttagagttcaagaggaatttagttttgttcttatttattgtggaaattttggctgaggtacttatttcaagcttacggctccagacatgtcagaaattcaaggacgtctgacaatcagaggctttagaaattaagtgataggcttacaaattaagtgatatgatctgtatgacactcttctccttgtgttctatgaatctgtccttgatgtataccctactcatcctgactgtgtctcacgagttcatatctgagtctgtgtccgttatatgtcctgtcattgtgccttatgtgtaatagatactaggtatttactgaatgaatactcagctcaaaggccactgtaaggctggatttgcaattacttgcctaatattagacatttagactctggtgatcatcagacctcagataaatggcctctttgatgttttattcttgtttaagaaccacagaagttttccaggctatgtcattttttccttcattagtttgcaattgtatttttgtgaattgtgtgaacatgatactaaatgaaatagtattaaatgttttgtttttcattgactgaattccatctcccaatgacatgattacatctttgaatactaaacgttttctgacaggccgcagttaatgattacctcctttccttcttcattcattgttcttcttcaagatgaacttacagtttccataaaacaagttattctgtggataatagaatgtttgtgtatgtgttggctttatttctcaccatgttattctcactattggaatttagggcaaacctaagaagaatgagagcctgtggagtgtagcaagaggtgttattagaatgttacgaaaaaactatggttgtgtccgagtggattttgcacagccattttccttaaaggtgagtgtctgttgaaatgaaaccatgttgaagcatctacctggagtcaggaggctccagtgctgactcttaacagcacctctctgtgtgacttggggctgcagtttgctctcctgtaaaatgaggactatgataaaggtggtgttgcctaaatttttccacaaaatttgcctattgacaaaggagagtgagtatctgtttttgggagatatcaggctcgacatttattgctattattatttttttttaattcatgaccatctaaggggaacaaaaaacaggtagaaggaataagttttagtatttgatagtacagtagggaaattatagttagcagtaatttattgtatatttcaaaataactaaaagagaagaattgtaatattcccaaccccccacaaaaagctaagtgtttgaggtgatggttatcccaattaccctgattttatcattacacattgtatatagatatcaaaatatcacatgtaccccaaaatatgtacaagtattatatatcaattaaccccaaaagtcaaaaaaattttgagatggatgaatgaataaatgaataaataattgtaaaaaaataaaaattcatgggaatcttgcatgtgcctttataatacgtatttatatcttatgtttcaaaattgcctttaggggaaatttttccaaacacctcccctaaaagctgggtaactttttttttgagacggagtttcatgttcttgtagcccaggctggagtgcagtggcgaaatctcagctcactgcaacctctgccccctaggttcaagcaattctcctgcgtcagcctcctaagtagctgggattacaggtgcccgctaccacacctggctaatttttgtatttttagtagagacggggtttcaccatgttggccaggctggcctcaaacttctgacctcaggtgatctgcccgcctcctcatctgcctcccaaagtgctgggattacaggtgtgagccacttcgggcagccaaagctgggtgaatttgacaggataatgctctctatgtcctattgtgtagaatccagtacccacacatcccaaaggatagtgccatcctcattgaaagtctgggactggcaggtccctgaggcccttgctggtttaaattcttaaggtattcaaattgtaaaggagaaggagaaaaatgggtacagaagtagggagcccagggataaggagcagaaggtcccgcctctccttttattctacttcctggcttaactttaagagattccagctgctgtggtagatgcttgctaaccttaaatgctttgatcttttagaaccttatcatttcttgccactttctttctctttattttttaaatggtcacatagcttctctgtcttgttatgatttgcttttagattgaggttcttttgaaaataatgtgacccttgggttgccagatttttttaagtatgattatgtctatcatcactattaatgatattaagaatgtcagttttatgatgatgtgtgagaaaagttaaaaatttcttttaattttaaaatgattttgtttaagcatcctattctttgtgtcttcagtgttcaagttcagtttcatgcttagcagcattctgtgaatcattctggacttctgaagacagtttaggatatagaaagtatttcccatgctctatagcatggcttatttcataaacaactcccttttaacctttggaagatgaaattacattttatatgttggtgcgaaagtgagggtaaatgatcatgttgtgtttttttctccaggaatatttagaaagccaaagtcagaaaccggtgtctgctctactttccctggagcaagcgttgttaccagctatacttccttcaaggtaaaatttgtagaattgtttaatgctaatcaagctaccaaacatctgttatttcttgtattgcatagattaaccttattaaaaatgcgcacattcttattttcacttcttaagtaacttttctcttagtccaataagggatattgagatgacatgttttcatattagtttggaagtttttaattatttgcttataactttttgattttgtgactcatataggagttcttaattgatttatgctttttgaatgcttgaactgagtttttttggtagtaggttatgcatattgtatgtaagtgaaccatatgaaagaattttgctgtttgaatttttcggaattcaaattgaattcaaattgaattttaacttagagaaattgaatatgaagcctttcctttaatatgtggaagtgttgaattttcagaaatgttttaaaataaggcatcttgaattatgtattctttctgccagtggttttggaagtggtgtatcaacagactatttagtcataactaaataacatgttttggccaggccataatgtagtatattatggaggaaaataagccatagaaataatctgtttgtggtttaaatttaaaatatatagtaatttaaagtatctaattcatttaaagatcagttccacatggtttgccttgtaaaagagtgcggttgtgtgttgatttttgaaacactttttagacccagtgatgctgctgatgaaggtagagacacgtccattaatgagtccagaaatgcaacagatgaatccctacgaaggaggttgattgcaaatctggctgagcatattctattcagtaagtagaataccagactctttaattctacaatttagattcatcatggtatggtgcaaactatacagattttggaatctgctgaggttagtttccacttctattaattcacaggtggtgataggtaagttattagaaagaaggtgataccgtgaggatttgagataattggagttaaacatcttatacagtgtctggtaattagtaggtgttcatgtggtagttgttgtactttttaaaaatagttattattaaaatgatttgttgcctctgttgctcattaatgtgttttatttgaactacttgaactaatttgtggtacaacgtctagatttatagttgcatgtccatttctcctttgaccccatgagtttttgtaatgcagcaaatatgagtggccttgtataaagctttgccttagccgtggagaatacaaaatgaataattaatggttctgaactcaatgagcttatgttcttgtcagtgggatggttatacacaaataattaaagtgaacctagagaggagaggagtgtgagttcactgcaggaccagtcttctttaagttccaagctgtacaattgtgtactgttgtggaggctgctttaaaatcccaaggcagcacagtggttctttggtgctcagcctagttgtgatttctttcttgctgctctctgggccaggaccctgatgatactctccgcaccccctgccaatgctactggggaggtcttcctccctcaccttgctcctcccattctggaggcccctattcctgcttgactttgggtcagaggattctttctcaagcacatttgattatgtctctatcctgcttaaaatctggatacaaaaaataataccttattgttaaaaagtttgaaaataaagatgaaatagacagcaaaaacccattacccaaataaccagtggtgttgctgtgtagtctctaaaaatattttttacagttttgtattgtgcagctttccatttataacacaatattcatatttaaaaattctttataaatatttttaatagctatacatatagtactgagtacttttttttaacactggagacgacttcagttaccagtggtgaatgaagtattcttaaatttattgttatcttagaaaactcacaaatgttatcgtgatttaagattattccatgggtgttctcagatttgatttatataattataattttatgcaaattcagtgtaggggtgtgcactgtagtcattctcaaatgtgtgtctgtgcccttccttcttacctctgggtcaggttgaaagaggaaatacaaatatgctgagcacccattactgtggctcctgtgccgggccagtcattttattctcatgtggtctaatgagctaggtaggattttcattttatcagcaaggaaggtgaggccaggaatttaagtaatttgccccaagtcatgaagttagttagtgagtaaattaggatttgagcccagatttttctgacttcaagtcagctgttatccttctactacaccttcgttttaatatggtgaataccagcaagaagagtgaagtagagttgtagttataatagactgtaggtgggagcaagtaaaggaatatggaaatgggagatcccgtagaactaaaacagccttgtactttgggtcttgatatttcaataaaagtgtctcttcgtcattcagtaactaaattccactggcctggattgaagttggacctgtcttacttagcctgttgtatgatgaatagtgatctctaatgaggttccattataccagaacactgtagtattataacttccttttggtaaatcacacttatgggaaatgtgatagaagactttcacagttttaaatactcctgtttaaaatttaggtatttcaaaaaagttcagctatgtttgtaaaaagtgcaactaaaaaaagctttgtgtttgccttccagctgctagcaagtcctgtgccattatgtccacacacattgtggcttgcctgctcctctacagacacaggcaggtatgtctgcaatgtcacctgcagttaagtaggaagcaggagaatgttttgtcagctgctcagatacatactcaggggcagatttggcccactcatggctagtactatgaggtaaacagtaaaataagctgaggaccctgcccaaaggagcttatcaacttggtggaggaagaatctacatttaaaaacaaaacaaaacagactgcttaatatatatagtactaagctattgcttgattaatactgttaatgtaaataaggtctaactcattaacatgtggtatatattctgaaagcaaatgctttccaaatgaattgactggcattttagaacccaccctttatcctcccaaaatacatttgtctgagctttatgatggaagagaccacaccagtcttgttcagtcatattctcaacaccaaacagtgccaatatatataggcaccaaataaatatttgccagataaatatttaaaatgaaggaaattttgctaaaaaataagcactccttacttgcagatggcttttttcctcccccttgtgatcttatgtgtgcatatcatgtgtcatggctagagtgttcactagcatgtgtaataccttgacattcctggaggaccagctttttaagtagttttctgactcgctctgcattggtgttctggccctacttctctatgtgctagcagtgagctgcaggaaagccaaggcaagaagaaagtcttggaatgttggcctctcagctttttggaaagtaggttttccaccatcatgataccgtttccatctttctttagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcataggtatgtcagaccttgaaatattttcagtaattttcatggaaaataaaaaggcctaaaacacatagtggtatctgctttaatttgggtgatagccccttgcttgagggaatgtggctataattttagttttcaaacgaaaatgtaatctcctgttatatcactgtttacaatgagtagttactgaattaccgtttgtatagcattacccttatttttcaggagaggtagagtgtcttgctatccaggagctgaaaatctaactgagatgagacatacacagagcagcaaagtgagtggagggcctcatttgtgattaagagaaaagagaggaaggagagaagagggggcttatgtctttggagaaggtttttgatgcagttgggatcatatggaatcataagaatttagtcttatgggatttacttaaatttctccaaatacaggccattatttttgatgtattaataatctcttaaggttattcaagccagttgaagtttttcttggagactttgagctggcaacttcattgttgcttttcttttcttagcttgcagcctttatgcagttctgaacaagaggggactggggggtcccactagcaccccacctaacctgatcagccaggagcagctggtgcggaaggcggccagcctgtgctaccttctctccaatgaaggcaccatctcactggtgagtgaagacccttcagtgttgcctggtctcttttcttgagcgttttcatgaaaacgtaaccccccgtgtttttctgttagataatgtttgacccattgttgatactttatattccactcctggatttcagtgaccaccaaaccaatatgtcatttactatcttggtttgctctctgggtcactgctccactgaatctatcctgcagctatgagccatgataggacagattgctcaatgttctcaggcagtcacatccaaagctagtgagcacgtattgtgtgtgaggcactgggtcagtggtttgtaaataattaacacacccacctgtagatgatggaaccacagatggtaagagtcagaagcagccttaaaaatcatctgctcggccaggtgcggtggctcacgcctgtaatctcagcactttgggaggctgaggcgggcagatcacgaggtcaggagatcgagaccatcctggctaacgtggagaaaccccgtctctactaaaaatacaaaaaaaaattagccaggtgtagtagcacctgcctgtagtcccagctactttggaggctgaggcaggaggatcacttgaacctgggaggcggaggatgcagtgagctgagattgcgccactgttctccagcctgagtgacagagcgagactccatctcaaaaaaaaaaaaaaaaaaaaaagaaaatcatctgctcaacacctctcaatttacaaatgaggaaaaccaagcccagcagtactaatgagcatttatattatatacccaaacacttttaggcatttatcatgagacatatgtattaagtagtagatttccttcttctcaaagtcatcaacatgtagaattcatattttatttgaattccgatattttcttttgtatgaatgtgtcttgagatgttgacgagatgagtgcatgtgttaggagtaccgccccctccatgccatgctttgtgtgaggtgtgctcgtggtcctgttggggaatctgttttgcatgaaccttgggtggtgggatggaagggatgagggcagataagacttccaagattgaccttgtcctttctctggtatttatttacttattccaatgttgcctttcctcaatcaccacttttgtgtacagtatttattataagataactattgcagagaaattgtcttataaagtaaaagaaatagatggtggggggctgataagcagtcttcagagggctttccacactgtgggtgtggggagagaattgagctcaggagtaccccttgcaggtggtgcagtgttgggtgctctgccaactttaagtgacagaggtcttttcttgggctttgtccatgaccgtgtgtgtatcaagacagtgggcactgggaatcacaggacttgctcagtggggtgatggaggtaacagaatggaaggctttggcagctaccttgtaccatgttttctgcgtttaaaaaaagaagtataaaatggtggcatttatatttaaatgaattttcttttcagccttgccagacattttaccaagtctgccatgaaacagtaggaaagtttatccagtatggcattcttacagtggcagaggtaagaggtagagctttcctttatttgtctttatacttactcttcattcccttaccttttctacaacttatttaaaccaataattggccaggcatggtggcatgtgcctgtaatcccagcactttgggaggtgaaggcgggtggatcacctgaggtcaggagtacgagagcagcctggccaacatggcacaaccctgtctttcctaaaaatacaaaaattagctgggcatggtggcacacacctgtaatcccagctacttgggaggctgaggcacaagaatcggagtggtggaggtttcagtgagccaagatcatgtcagtgcactccagcctgggtgacagagtgagactcagtctcataaataagtaaacaaaccaataattacatttggtatatagtcatgttcagatgtagagaagcaaaaaataagaatggggagaataagtgtcctttgggattatttaatattttggttcctatttgtaagttgtgttgtctaaaacatatcctgacttgattccgtgacatccagaagtttgtatgctaagttgagacgttacacctatcagtatgcttgcagcttgcacatctgcagttttcatttttgagattctattttaattttggtagatttagtgtagaggagatggcctggaaaagtacatatcaatttagaacatgtgggggtacttttatttgatctttctttctggtagctctcccctcccacctgccctttcttttgtctcctgctgtctcctgactgagtttgagtttggaagggaagtggactgacattatgagtggctaaacagagaagagccaggaagggtcagagaagaggactgccttgtcctggctccactgggcagttgtttgtgtgccagtgcatatttatttactatccatcatctccttggcctagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtacttgggtgaagaattctggtggatattacagggatatgttgaggtttatgctgcagtgagatcagctggagtcctggtgatgtcttcttatctaaagaatcccccaactagctctggtaccttctgtgtggtaaagacactcaaactgtttgagttgaattaatagcattttaagtagaaaaggaaaggagagtctgaaaagtcaggaagatgaatgtcataggtgagactttcaccatccttttatgaaatacacaggtgcatacctgtttacctacacctgcacccctcatgaggcagcagttttgctattgagctgccactgacctggctgctctttttgagtcactcttgctgtccctcccaaaatttcatatattaagctctttgctgtcaattaaaacaaataccattataggagaaaattgagattaaaaaaaaagtccctgatttagaaaaatcaattttgtctaatttataattttagaacttagtaataatgacccgtcttttctgaatactctaagaggattactcttttttgacatttagaaattgtcttctttttcacttgggtggtattagtttagtttcaagatggggcagtgatcttgctttcacactccagaggggcttgaccgaaccagtgtgttttgggtaggtacagtgagagcctctcggccataaagcaccgccgtcacagtggccatcattccccacagtgctggactgtgggcaaaggccatttaggggaggcagggaataggtgctgcagaaggagtgagataatcttggtggtctcccattggtctttctgcagacttgagtgactgttgagcccaggcttcaaaccatggagggcctggtcttaggagcggctatttttagtgatgacagcgtatcacaagtagggcattcatttatgaaaatttcttctaggtggctgttcaatgaacacaagcctcaaataccataaaaaagtgaatgattacaataaagaatgtgtttgaaagccagcagttgtttccagcagagattctctgcatgaggggcagggggccgctttcatgtagtgctgatgtgagtggccatcttctcacgttactggctctccagagaaagtcctctgtccacttgccttgtgtctcttgtcccttcctcatgacttcatctcctgcttttgcacactcaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatggtatgttaagtcactatttattcttttaaaatcttttttttttttttggatttcagaaatttgctaattgtagaaaattggaaaaatgcaaacttatcctgacctctaacaccgtagagtattactattatcttctttggcatgttatataagttgaatgatacagttatacactcttacatctggcttttttcacgtaacgttatttttgagattcatacatgttgcatatagttgttcctttgttcttgttgtgtagtgttccattgtataaatatttaccacattttattcattctgctgttgatggatatttgggttgttttcagttcacagctgctttgaacagtgttgctatgagcatatttgaatatgtcttttggtgaatatctgtgcccctatatacagcatgtatgtgggaacagaattgcctggttgtaggctctgggtatattcggctttggtagataccaccaaacagtttccaagggattatacctacttgtactcccacaacagtgtgtgtttcacttgctgcacatcttggccagcgctccgtcttctttgttttaggaggtgtgattggggtgggagtactattttatacagttgtagtttggagctgaaactacttgaatcactttttagagccaaaaggaatctaatctaatcacctcattttatagttgaggaaaataccccaggtcacactgctaattaatgggtaatctcgggatcagaattcttgtttcctaacctcgaggcctgtgctttctctggttccactgatgtacaatcatctgtgtaccactgggtagcttaaagaatatttatacactatctcatttgattctcacaacagtcctggaaaatggatgttttaggtatttctacttccctctgttctccctatttctctctctacaccctcttttctttccccttccctttcttatttacctcaatataggacaaagtaggtgtaagcaaagtaggtttaacagcaagtcatctgagaactcacaggttgccagtggtggaagtggggccccacccttgtctgctgacttgccctcattctcttcctagctccccatactttcttactgtggctgctgggattgtcatgatttgttgagcgtaaccatttgacagggttttattctctctcttcagctgagagtgccacatattgtcttgtgaagaatgctgtgaaaatgtttaaggatattggggtaggtgtccaccatttatggtataaaagctatctcaacttctgttctctttagatctagtctgtttgagctacctttgtggtggggtggaccccgagagaagcagtttctgctggcttaatgataaaggcatttttgggaacggggcatgtaggatgggttgggctatgttgaaggtgaagcccatcagtgtagatttatttgaatgttctggaattttactggtttcacatttattcccaagctgctatatataactggtatcaatatgtttcaaggtgctacaggttgaatatcccttatccaaaatgttgggaccagaaatgttttggatttcagatttttttgggattttggaatatgtgcattagacttacaggttgagcgtctctaatccaaaacccaaaatgctctgatgagcatgtcttttgagcatcatattgatgttcaaaatctttcagattttggagtacttcagatttttgattttgggattaggaccacaatcttggctcttaattattccatatgatttttagttactttcttgtatttttacaagaatttctagaagtatcctcaggtgtcctttaccttcatgattcatggggaatgtaagttctatagagatactagcggccttttgtgagggactgtttgtgactttattctaagtcattttagagtatagttatgttggttgaaattttagaatatagttaatgttggttgaactccatcaaaacaaaaaaaaaaaaatagaaaaagaaaaccttgtttgcattggaaactcccacctcatgctcacacacacccaggcatacatttacttaatgccaggatatggttttttgtgcctttactgccttctgaggtctaggacaccacaggtcaggttggtaatggaaattttttttattttttattttttgcctttaatttaatctaaaccttttctttaggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacata

SEQ ID NO: 3 - RNA, 6372 nt, Homo sapiens
  source 1..6372: mol_type = mRNA
gcgccactgcagctggcattggccgggactggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacata

SEQ ID NO: 4 - RNA, 5664 nt, Homo sapiens
  source 1..5664: mol_type = mRNA
gaagctggagctgctagggtgcgaactgccagggcaggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtacttgggtgaagaattctggtggatattacagggatatgttgaggtttatgctgcagtgagatcagctggagtcctggtgatgtcttcttatctaaagaatcccccaactagctctggtaccttctgtgtggtaaagacactcaaactgtttgagttgaattaatagcattttaagtagaaaaggaaaggagagtctgaaaagtcaggaagatgaatgtcataggtgagactttcaccatccttttatgaaatacacaggtgcatacctgtttacctacacctgcacccctcatgaggcagcagttttgctattgagctgccactgacctggctgctctttttgagtcactcttgctgtccctcccaaaatttcatatattaagctctttgctgtcaattaaaacaaataccattataggagaaaattgagattaaaaaaaaagtccctgatttagaaaaatcaattttgtctaatttataattttagaacttagtaataatgacccgtcttttctgaatactctaagaggattactcttttttgacatttagaaattgtcttctttttcacttgggtggtattagtttagtttcaagatggggcagtgatcttgctttcacactccagaggggcttgaccgaaccagtgtgttttgggtaggtacagtgagagcctctcggccataaagcaccgccgtcacagtggccatcattccccacagtgctggactgtgggcaaaggccatttaggggaggcagggaataggtgctgcagaaggagtgagataatcttggtggtctcccattggtctttctgcagacttgagtgactgttgagcccaggcttcaaaccatggagggcctggtcttaggagcggctatttttagtgatgacagcgtatcacaagtagggcattcatttatgaaaatttcttctaggtggctgttcaatgaacacaagcctcaaataccataaaaaagtgaatgattacaataaagaatgtgtttgaaagccagcagttgtttccagcagagattctctgcatgaggggcagggggccgctttcatgtagtgctgatgtgagtggccatcttctcacgttactggctctccagagaaagtcctctgtccacttgccttgtgtctcttgtcccttcctcatgacttcatctcctgcttttgcacactcaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatggtatgttaagtcactatttattcttttaaaatcttttttttttttttggatttcagaaatttgctaattgtagaaaattggaaaaatgcaaacttatcctgacctctaacaccgtagagtattactattatcttctttggcatgttatataagttgaatgatacagttatacactcttacatctggcttttttcacgtaacgttatttttgagattcatacatgttgcatatagttgttcctttgttcttgttgtgtagtgttccattgtataaatatttaccacattttattcattctgctgttgatggatatttgggttgttttcagttcacagctgctttgaacagtgttgctatgagcatatttgaatatgtcttttggtgaatatctgtgcccctatatacagcatgtatgtgggaacagaattgcctggttgtaggctctgggtatattcggctttggtagataccaccaaacagtttccaagggattatacctacttgtactcccacaacagtgtgtgtttcacttgctgcacatcttggccagcgctccgtcttctttgttttaggaggtgtgattggggtgggagtactattttatacagttgtagtttggagctgaaactacttgaatcactttttagagccaaaaggaatctaatctaatcacctcattttatagttgaggaaaataccccaggtcacactgctaattaatgggtaatctcgggatcagaattcttgtttcctaacctcgaggcctgtgctttctctggttccactgatgtacaatcatctgtgtaccactgggtagcttaaagaatatttatacactatctcatttgattctcacaacagtcctggaaaatggatgttttaggtatttctacttccctctgttctccctatttctctctctacaccctcttttctttccccttccctttcttatttacctcaatataggacaaagtaggtgtaagcaaagtaggtttaacagcaagtcatctgagaactcacaggttgccagtggtggaagtggggccccacccttgtctgctgacttgccctcattctcttcctagctccccatactttcttactgtggctgctgggattgtcatgatttgttgagcgtaaccatttgacagggttttattctctctcttcagctgagagtgccacatattgtcttgtgaagaatgctgtgaaaatgtttaaggatattggggtaggtgtccaccatttatggtataaaagctatctcaacttctgttctctttagatctagtctgtttgagctacctttgtggtggggtggaccccgagagaagcagtttctgctggcttaatgataaaggcatttttgggaacggggcatgtaggatgggttgggctatgttgaaggtgaagcccatcagtgtagatttatttgaatgttctggaattttactggtttcacatttattcccaagctgctatatataactggtatcaatatgtttcaaggtgctacaggttgaatatcccttatccaaaatgttgggaccagaaatgttttggatttcagatttttttgggattttggaatatgtgcattagacttacaggttgagcgtctctaatccaaaacccaaaatgctctgatgagcatgtcttttgagcatcatattgatgttcaaaatctttcagattttggagtacttcagatttttgattttgggattaggaccacaatcttggctcttaattattccatatgatttttagttactttcttgtatttttacaagaatttctagaagtatcctcaggtgtcctttaccttcatgattcatggggaatgtaagttctatagagatactagcggccttttgtgagggactgtttgtgactttattctaagtcattttagagtatagttatgttggttgaaattttagaatatagttaatgttggttgaactccatcaaaacaaaaaaaaaaaaa

SEQ ID NO: 5 - RNA, 6368 nt, Homo sapiens
  source 1..6368: mol_type = mRNA
gcgccactgcagctggcattggccgggactggaagtgcgggcttctgcagcagccgaagctggagctgctaggcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacata

SEQ ID NO: 6 - RNA, 4918 nt, Homo sapiens
  source 1..4918: mol_type = mRNA
ggccactgcagctggcattggccgggactggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaactttcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccggtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctcccaagttctgcatttaaaatatattttttctttataggaattgatgtataccatgaagtcattgtcagttgtagtagctctgatgttgaatgagatatcatgttttagcattccattttactgactagggtagaagaacactcttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaaaaaaaaaaaaaaaa

SEQ ID NO: 7 - RNA, 3490 nt, Homo sapiens
  source 1..3490: mol_type = mRNA
agcgggctggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatatgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaacaccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtacaagcttctggctcaagagttgaaggtgccatcgcagggtcaggcctgccctgtcccgaagtgatctcctggaagacaagtgccttctccatccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaactcaaaaaaaaaaaaaaaaa

SEQ ID NO: 8 - RNA, 2855 nt, Homo sapiens
  source 1..2855: mol_type = mRNA
aactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttactagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttcctgaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccgtcgcagggtcaggcctgccctgtcccgaggtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagaggtcttttaaggcaaaaaaaaaaaaaaaaaaaaa

SEQ ID NO: 9 - RNA, 6372 nt, Homo sapiens
  source 1..6372: mol_type = mRNA
gcgccactgcagctggcattggccgggactggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacata

SEQ ID NO: 10 - RNA, 5664 nt, Homo sapiens
  source 1..5664: mol_type = mRNA
gaagctggagctgctagggtgcgaactgccagggcaggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtacttgggtgaagaattctggtggatattacagggatatgttgaggtttatgctgcagtgagatcagctggagtcctggtgatgtcttcttatctaaagaatcccccaactagctctggtaccttctgtgtggtaaagacactcaaactgtttgagttgaattaatagcattttaagtagaaaaggaaaggagagtctgaaaagtcaggaagatgaatgtcataggtgagactttcaccatccttttatgaaatacacaggtgcatacctgtttacctacacctgcacccctcatgaggcagcagttttgctattgagctgccactgacctggctgctctttttgagtcactcttgctgtccctcccaaaatttcatatattaagctctttgctgtcaattaaaacaaataccattataggagaaaattgagattaaaaaaaaagtccctgatttagaaaaatcaattttgtctaatttataattttagaacttagtaataatgacccgtcttttctgaatactctaagaggattactcttttttgacatttagaaattgtcttctttttcacttgggtggtattagtttagtttcaagatggggcagtgatcttgctttcacactccagaggggcttgaccgaaccagtgtgttttgggtaggtacagtgagagcctctcggccataaagcaccgccgtcacagtggccatcattccccacagtgctggactgtgggcaaaggccatttaggggaggcagggaataggtgctgcagaaggagtgagataatcttggtggtctcccattggtctttctgcagacttgagtgactgttgagcccaggcttcaaaccatggagggcctggtcttaggagcggctatttttagtgatgacagcgtatcacaagtagggcattcatttatgaaaatttcttctaggtggctgttcaatgaacacaagcctcaaataccataaaaaagtgaatgattacaataaagaatgtgtttgaaagccagcagttgtttccagcagagattctctgcatgaggggcagggggccgctttcatgtagtgctgatgtgagtggccatcttctcacgttactggctctccagagaaagtcctctgtccacttgccttgtgtctcttgtcccttcctcatgacttcatctcctgcttttgcacactcaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatggtatgttaagtcactatttattcttttaaaatcttttttttttttttggatttcagaaatttgctaattgtagaaaattggaaaaatgcaaacttatcctgacctctaacaccgtagagtattactattatcttctttggcatgttatataagttgaatgatacagttatacactcttacatctggcttttttcacgtaacgttatttttgagattcatacatgttgcatatagttgttcctttgttcttgttgtgtagtgttccattgtataaatatttaccacattttattcattctgctgttgatggatatttgggttgttttcagttcacagctgctttgaacagtgttgctatgagcatatttgaatatgtcttttggtgaatatctgtgcccctatatacagcatgtatgtgggaacagaattgcctggttgtaggctctgggtatattcggctttggtagataccaccaaacagtttccaagggattatacctacttgtactcccacaacagtgtgtgtttcacttgctgcacatcttggccagcgctccgtcttctttgttttaggaggtgtgattggggtgggagtactattttatacagttgtagtttggagctgaaactacttgaatcactttttagagccaaaaggaatctaatctaatcacctcattttatagttgaggaaaataccccaggtcacactgctaattaatgggtaatctcgggatcagaattcttgtttcctaacctcgaggcctgtgctttctctggttccactgatgtacaatcatctgtgtaccactgggtagcttaaagaatatttatacactatctcatttgattctcacaacagtcctggaaaatggatgttttaggtatttctacttccctctgttctccctatttctctctctacaccctcttttctttccccttccctttcttatttacctcaatataggacaaagtaggtgtaagcaaagtaggtttaacagcaagtcatctgagaactcacaggttgccagtggtggaagtggggccccacccttgtctgctgacttgccctcattctcttcctagctccccatactttcttactgtggctgctgggattgtcatgatttgttgagcgtaaccatttgacagggttttattctctctcttcagctgagagtgccacatattgtcttgtgaagaatgctgtgaaaatgtttaaggatattggggtaggtgtccaccatttatggtataaaagctatctcaacttctgttctctttagatctagtctgtttgagctacctttgtggtggggtggaccccgagagaagcagtttctgctggcttaatgataaaggcatttttgggaacggggcatgtaggatgggttgggctatgttgaaggtgaagcccatcagtgtagatttatttgaatgttctggaattttactggtttcacatttattcccaagctgctatatataactggtatcaatatgtttcaaggtgctacaggttgaatatcccttatccaaaatgttgggaccagaaatgttttggatttcagatttttttgggattttggaatatgtgcattagacttacaggttgagcgtctctaatccaaaacccaaaatgctctgatgagcatgtcttttgagcatcatattgatgttcaaaatctttcagattttggagtacttcagatttttgattttgggattaggaccacaatcttggctcttaattattccatatgatttttagttactttcttgtatttttacaagaatttctagaagtatcctcaggtgtcctttaccttcatgattcatggggaatgtaagttctatagagatactagcggccttttgtgagggactgtttgtgactttattctaagtcattttagagtatagttatgttggttgaaattttagaatatagttaatgttggttgaactccatcaaaacaaaaaaaaaaaaa

SEQ ID NO: 11 - RNA, 6368 nt, Homo sapiens
  source 1..6368: mol_type = mRNA
gcgccactgcagctggcattggccgggactggaagtgcgggcttctgcagcagccgaagctggagctgctaggcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacata

SEQ ID NO: 12 - RNA, 4918 nt, Homo sapiens
  source 1..4918: mol_type = mRNA
ggccactgcagctggcattggccgggactggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaactttcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccggtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctcccaagttctgcatttaaaatatattttttctttataggaattgatgtataccatgaagtcattgtcagttgtagtagctctgatgttgaatgagatatcatgttttagcattccattttactgactagggtagaagaacactcttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaaaaaaaaaaaaaaaa

SEQ ID NO: 13 - RNA, 3490 nt, Homo sapiens
  source 1..3490: mol_type = mRNA
agcgggctggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatatgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaacaccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtacaagcttctggctcaagagttgaaggtgccatcgcagggtcaggcctgccctgtcccgaagtgatctcctggaagacaagtgccttctccatccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaactcaaaaaaaaaaaaaaaaa

SEQ ID NO: 14 - RNA, 2855 nt, Homo sapiens
  source 1..2855: mol_type = mRNA
aactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttactagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttcctgaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccgtcgcagggtcaggcctgccctgtcccgaggtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagaggtcttttaaggcaaaaaaaaaaaaaaaaaaaaa

SEQ ID NO: 15 - DNA, 6372 bp, Homo sapiens
  source 1..6372: mol_type = genomic DNA
gcgccactgcagctggcattggccgggactggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacata

SEQ ID NO: 16 - DNA, 5664 bp, Homo sapiens
  source 1..5664: mol_type = genomic DNA
gaagctggagctgctagggtgcgaactgccagggcaggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtacttgggtgaagaattctggtggatattacagggatatgttgaggtttatgctgcagtgagatcagctggagtcctggtgatgtcttcttatctaaagaatcccccaactagctctggtaccttctgtgtggtaaagacactcaaactgtttgagttgaattaatagcattttaagtagaaaaggaaaggagagtctgaaaagtcaggaagatgaatgtcataggtgagactttcaccatccttttatgaaatacacaggtgcatacctgtttacctacacctgcacccctcatgaggcagcagttttgctattgagctgccactgacctggctgctctttttgagtcactcttgctgtccctcccaaaatttcatatattaagctctttgctgtcaattaaaacaaataccattataggagaaaattgagattaaaaaaaaagtccctgatttagaaaaatcaattttgtctaatttataattttagaacttagtaataatgacccgtcttttctgaatactctaagaggattactcttttttgacatttagaaattgtcttctttttcacttgggtggtattagtttagtttcaagatggggcagtgatcttgctttcacactccagaggggcttgaccgaaccagtgtgttttgggtaggtacagtgagagcctctcggccataaagcaccgccgtcacagtggccatcattccccacagtgctggactgtgggcaaaggccatttaggggaggcagggaataggtgctgcagaaggagtgagataatcttggtggtctcccattggtctttctgcagacttgagtgactgttgagcccaggcttcaaaccatggagggcctggtcttaggagcggctatttttagtgatgacagcgtatcacaagtagggcattcatttatgaaaatttcttctaggtggctgttcaatgaacacaagcctcaaataccataaaaaagtgaatgattacaataaagaatgtgtttgaaagccagcagttgtttccagcagagattctctgcatgaggggcagggggccgctttcatgtagtgctgatgtgagtggccatcttctcacgttactggctctccagagaaagtcctctgtccacttgccttgtgtctcttgtcccttcctcatgacttcatctcctgcttttgcacactcaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatggtatgttaagtcactatttattcttttaaaatcttttttttttttttggatttcagaaatttgctaattgtagaaaattggaaaaatgcaaacttatcctgacctctaacaccgtagagtattactattatcttctttggcatgttatataagttgaatgatacagttatacactcttacatctggcttttttcacgtaacgttatttttgagattcatacatgttgcatatagttgttcctttgttcttgttgtgtagtgttccattgtataaatatttaccacattttattcattctgctgttgatggatatttgggttgttttcagttcacagctgctttgaacagtgttgctatgagcatatttgaatatgtcttttggtgaatatctgtgcccctatatacagcatgtatgtgggaacagaattgcctggttgtaggctctgggtatattcggctttggtagataccaccaaacagtttccaagggattatacctacttgtactcccacaacagtgtgtgtttcacttgctgcacatcttggccagcgctccgtcttctttgttttaggaggtgtgattggggtgggagtactattttatacagttgtagtttggagctgaaactacttgaatcactttttagagccaaaaggaatctaatctaatcacctcattttatagttgaggaaaataccccaggtcacactgctaattaatgggtaatctcgggatcagaattcttgtttcctaacctcgaggcctgtgctttctctggttccactgatgtacaatcatctgtgtaccactgggtagcttaaagaatatttatacactatctcatttgattctcacaacagtcctggaaaatggatgttttaggtatttctacttccctctgttctccctatttctctctctacaccctcttttctttccccttccctttcttatttacctcaatataggacaaagtaggtgtaagcaaagtaggtttaacagcaagtcatctgagaactcacaggttgccagtggtggaagtggggccccacccttgtctgctgacttgccctcattctcttcctagctccccatactttcttactgtggctgctgggattgtcatgatttgttgagcgtaaccatttgacagggttttattctctctcttcagctgagagtgccacatattgtcttgtgaagaatgctgtgaaaatgtttaaggatattggggtaggtgtccaccatttatggtataaaagctatctcaacttctgttctctttagatctagtctgtttgagctacctttgtggtggggtggaccccgagagaagcagtttctgctggcttaatgataaaggcatttttgggaacggggcatgtaggatgggttgggctatgttgaaggtgaagcccatcagtgtagatttatttgaatgttctggaattttactggtttcacatttattcccaagctgctatatataactggtatcaatatgtttcaaggtgctacaggttgaatatcccttatccaaaatgttgggaccagaaatgttttggatttcagatttttttgggattttggaatatgtgcattagacttacaggttgagcgtctctaatccaaaacccaaaatgctctgatgagcatgtcttttgagcatcatattgatgttcaaaatctttcagattttggagtacttcagatttttgattttgggattaggaccacaatcttggctcttaattattccatatgatttttagttactttcttgtatttttacaagaatttctagaagtatcctcaggtgtcctttaccttcatgattcatggggaatgtaagttctatagagatactagcggccttttgtgagggactgtttgtgactttattctaagtcattttagagtatagttatgttggttgaaattttagaatatagttaatgttggttgaactccatcaaaacaaaaaaaaaaaaa

SEQ ID NO: 17 - DNA, 6368 bp, Homo sapiens
  source 1..6368: mol_type = genomic DNA
gcgccactgcagctggcattggccgggactggaagtgcgggcttctgcagcagccgaagctggagctgctaggcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacata

SEQ ID NO: 18 - DNA, 4918 bp, Homo sapiens
  source 1..4918: mol_type = genomic DNA
ggccactgcagctggcattggccgggactggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaactttcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccggtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctcccaagttctgcatttaaaatatattttttctttataggaattgatgtataccatgaagtcattgtcagttgtagtagctctgatgttgaatgagatatcatgttttagcattccattttactgactagggtagaagaacactcttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaaaaaaaaaaaaaaaa

SEQ ID NO: 19 - DNA, 3490 bp, Homo sapiens
  source 1..3490: mol_type = genomic DNA
agcgggctggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatatgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaacaccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtacaagcttctggctcaagagttgaaggtgccatcgcagggtcaggcctgccctgtcccgaagtgatctcctggaagacaagtgccttctccatccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaactcaaaaaaaaaaaaaaaaa

SEQ ID NO: 20 - DNA, 2855 bp, Homo sapiens
  source 1..2855: mol_type = genomic DNA
aactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccatcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttactagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttcctgaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccgtcgcagggtcaggcctgccctgtcccgaggtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagaggtcttttaaggcaaaaaaaaaaaaaaaaaaaaa

SEQ ID NO: 21 - DNA, 6372 bp, Homo sapiens
  source 1..6372: mol_type = genomic DNA
gcgccactgcagctggcattggccgggactggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacata

SEQ ID NO: 22 - DNA, 5664 bp, Homo sapiens
  source 1..5664: mol_type = genomic DNA
gaagctggagctgctagggtgcgaactgccagggcaggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtacttgggtgaagaattctggtggatattacagggatatgttgaggtttatgctgcagtgagatcagctggagtcctggtgatgtcttcttatctaaagaatcccccaactagctctggtaccttctgtgtggtaaagacactcaaactgtttgagttgaattaatagcattttaagtagaaaaggaaaggagagtctgaaaagtcaggaagatgaatgtcataggtgagactttcaccatccttttatgaaatacacaggtgcatacctgtttacctacacctgcacccctcatgaggcagcagttttgctattgagctgccactgacctggctgctctttttgagtcactcttgctgtccctcccaaaatttcatatattaagctctttgctgtcaattaaaacaaataccattataggagaaaattgagattaaaaaaaaagtccctgatttagaaaaatcaattttgtctaatttataattttagaacttagtaataatgacccgtcttttctgaatactctaagaggattactcttttttgacatttagaaattgtcttctttttcacttgggtggtattagtttagtttcaagatggggcagtgatcttgctttcacactccagaggggcttgaccgaaccagtgtgttttgggtaggtacagtgagagcctctcggccataaagcaccgccgtcacagtggccatcattccccacagtgctggactgtgggcaaaggccatttaggggaggcagggaataggtgctgcagaaggagtgagataatcttggtggtctcccattggtctttctgcagacttgagtgactgttgagcccaggcttcaaaccatggagggcctggtcttaggagcggctatttttagtgatgacagcgtatcacaagtagggcattcatttatgaaaatttcttctaggtggctgttcaatgaacacaagcctcaaataccataaaaaagtgaatgattacaataaagaatgtgtttgaaagccagcagttgtttccagcagagattctctgcatgaggggcagggggccgctttcatgtagtgctgatgtgagtggccatcttctcacgttactggctctccagagaaagtcctctgtccacttgccttgtgtctcttgtcccttcctcatgacttcatctcctgcttttgcacactcaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatggtatgttaagtcactatttattcttttaaaatcttttttttttttttggatttcagaaatttgctaattgtagaaaattggaaaaatgcaaacttatcctgacctctaacaccgtagagtattactattatcttctttggcatgttatataagttgaatgatacagttatacactcttacatctggcttttttcacgtaacgttatttttgagattcatacatgttgcatatagttgttcctttgttcttgttgtgtagtgttccattgtataaatatttaccacattttattcattctgctgttgatggatatttgggttgttttcagttcacagctgctttgaacagtgttgctatgagcatatttgaatatgtcttttggtgaatatctgtgcccctatatacagcatgtatgtgggaacagaattgcctggttgtaggctctgggtatattcggctttggtagataccaccaaacagtttccaagggattatacctacttgtactcccacaacagtgtgtgtttcacttgctgcacatcttggccagcgctccgtcttctttgttttaggaggtgtgattggggtgggagtactattttatacagttgtagtttggagctgaaactacttgaatcactttttagagccaaaaggaatctaatctaatcacctcattttatagttgaggaaaataccccaggtcacactgctaattaatgggtaatctcgggatcagaattcttgtttcctaacctcgaggcctgtgctttctctggttccactgatgtacaatcatctgtgtaccactgggtagcttaaagaatatttatacactatctcatttgattctcacaacagtcctggaaaatggatgttttaggtatttctacttccctctgttctccctatttctctctctacaccctcttttctttccccttccctttcttatttacctcaatataggacaaagtaggtgtaagcaaagtaggtttaacagcaagtcatctgagaactcacaggttgccagtggtggaagtggggccccacccttgtctgctgacttgccctcattctcttcctagctccccatactttcttactgtggctgctgggattgtcatgatttgttgagcgtaaccatttgacagggttttattctctctcttcagctgagagtgccacatattgtcttgtgaagaatgctgtgaaaatgtttaaggatattggggtaggtgtccaccatttatggtataaaagctatctcaacttctgttctctttagatctagtctgtttgagctacctttgtggtggggtggaccccgagagaagcagtttctgctggcttaatgataaaggcatttttgggaacggggcatgtaggatgggttgggctatgttgaaggtgaagcccatcagtgtagatttatttgaatgttctggaattttactggtttcacatttattcccaagctgctatatataactggtatcaatatgtttcaaggtgctacaggttgaatatcccttatccaaaatgttgggaccagaaatgttttggatttcagatttttttgggattttggaatatgtgcattagacttacaggttgagcgtctctaatccaaaacccaaaatgctctgatgagcatgtcttttgagcatcatattgatgttcaaaatctttcagattttggagtacttcagatttttgattttgggattaggaccacaatcttggctcttaattattccatatgatttttagttactttcttgtatttttacaagaatttctagaagtatcctcaggtgtcctttaccttcatgattcatggggaatgtaagttctatagagatactagcggccttttgtgagggactgtttgtgactttattctaagtcattttagagtatagttatgttggttgaaattttagaatatagttaatgttggttgaactccatcaaaacaaaaaaaaaaaaa

SEQ ID NO: 23 - DNA, 6368 bp, Homo sapiens
  source 1..6368: mol_type = genomic DNA
gcgccactgcagctggcattggccgggactggaagtgcgggcttctgcagcagccgaagctggagctgctaggcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctctcaagttctgcatttaaaatatattttttctttataggaattgatgtataccatgaagtcattgtcagttgtagtagctctgatgttgaatgagatatcatgttttagcattccattttactgactagggtagaagaacacttttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaacaaaaaaacgaaagcatcttcacaatgaagcttccagatagcaccgttttgctaaaagatacattctcattgttttccaacagtgatggcttccacataaggttaaacaaactaggtgcttgtaaataatttattacagtttactctatcgcatttctgtaacatgaaatgcatgcccttcttcaggggaagactgtggtcaagttaaaaaaaaaaaacaatattaaacaacatgaaactgcagtctgtttttgaaaatgagaatgtcctaagtgattcagaagagaggagggaagttgtgcactctgaaaatgcatgaaaaacaaaggcaaaaactagtgggaaatgtgtagaactgttaactgagatggcttcgagtcttccttctggaatctgttaaatttcacaaagtcatgagggtaaatggagaaaatatttctgggattacaatgaatgtaagcccaaattgtggaattgccagtaacctggatggggaaaagcatttcccatagcactccatgtaatatgagtgctctgtgagatgttcatcagtgttttatagaaatggtgttgctgggaaaccaagtttgcacctggaaacttacaatgcactttagcgcagtaagggcttggcatccggtagtgaaaaactgtctaacccagcattgcccaaactattttgacaccaggacctttttctcctttgggatacttatgaacctctcactaatgtcctgtggagaacattttgggaaacactatgttagatagttctttaaggagacaaaacggtaatgaacagatagcactggggcagaatatgcatgcattttgtaacgtccagtgtggcgttgaatagatgtgtatttcctcccctgcagaaaataagcacagaaaattataatgtaggtgatcggagctctttcctttgatagagagaacagccccaatgatcctggctttttcactgaacgtatcagaatacatggatgaattggggtaaataaggttttaattcagatctagaagaaagtattgtacgtttgaatgcagatttttatccacagatagttgtagtgtttagacatgacaggacctatcgttgaggtttctaagacttactatgggctgtaaacctgttttttaaaactattttagaaacctgagacttgccgtctggcattttagtttaatacaaactaatgattgcatttgaaagagattcttgaccttatttctaaacgtctagagctctgaaatgtcttgatggaaggtattaaactatttgcctgttgtacaaagaaatgttaagactcgtgaaaagaattactataaggtactgtgaaataactgcgattttgtgagcaaaacatacttggaaatgctgattgatttttatgcttgttagtgtattgcaagaaacacagaaaatgtagttttgttttaataaaccaaaaattgaacata

SEQ ID NO: 24 - DNA, 4918 bp, Homo sapiens
  source 1..4918: mol_type = genomic DNA
ggccactgcagctggcattggccgggactggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaactttcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccggtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccatcgcagggtcaggcctgccctgtcccgaagtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaacccttcctgttttatgtctgtacctcgtccacccctcaggtaatacctgcctctcacaggtacagctgtttcttggaaatcctccaaccaaatagcagttttcctaacttgattagcttgagctgacagactgttagaatacagttctctggccacagctgatgagggctttctgtactgcacacagattgtgtactgcaccccagtccaggtgactggtacccactcgagttgtgccgtgcacaacctgtccagtatatgcatgtggtggccctactgactggtaatggttagaggcatttatggatttttagctttgaggaaaaaccatgacttttaacaaatttttatgggttatatgcctaaacccttatgccacatagtggtaaataattatgaaaaatggtctgttcataattggtaggtgccttttgtgagcagggagcataattattggtttattatggtaattatggtgattttttaaatatcatgtaatgttaaaacgttttctaacagtttactgttgcttatctccaagatattatggaattaagaatttttccagatgagtgttacatagattctttgaatttagtataaaagtactgagaattaagtttgtacttccataagcttggattttaaacactgatagtatctcatgagtaatgtgtgttttgggagagggagggatgctgattgatatttcacattgtatgaaataccatgtttgaaactcatagcaataatgctatgctgttgtgatccctcccaagttctgcatttaaaatatattttttctttataggaattgatgtataccatgaagtcattgtcagttgtagtagctctgatgttgaatgagatatcatgttttagcattccattttactgactagggtagaagaacactcttcttggctacatttggaggatacccagggagtcttgggtgttccttatctggggaagcaaacatttcactagtctctttttttcatcctttaaattgtaaattaaggattactcaagctcaccattattcaagattgggactcgcttcccagtcgacactctgccctgcctgtcattgctgcaaagagctgctgctttgccaacctaagcaaagaaaatacggcttctcttgcattattttcccttttggttggtttgttttctagaagtacgttcagatgctttggggaatgcaatgtatgatttgctagctctctcaccacttaactcactgtgaggataaatatgcatgctttttgtaattaactggtgctttgaaaatcttttttaagggagaaaaatctcaaccaaagttatgctcatccagacaagctgacctttgagttaatttcagcacaactcattcttcagtgcctcatgactgaaaacaaaaaaaaaaaaaaaaaaaa

SEQ ID NO: 25 - DNA, 3490 bp, Homo sapiens
  source 1..3490: mol_type = genomic DNA
agcgggctggaagtgcgggcttctgcagcagccgaagctggagctgctagggcagcagcggctcccctgttgtatggacattctgcacccgaaactgatagctgagtcctgaagttttatgttatgaaacagaagaactttcatcccagcacatgatttgggaattacactttgtgacatggatgaatatgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaacaccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttattagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttccagaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtacaagcttctggctcaagagttgaaggtgccatcgcagggtcaggcctgccctgtcccgaagtgatctcctggaagacaagtgccttctccatccatggatctgtgatcttcccagctctgcatcaacacagcagcctgcagataacacttggggggacctcagcctctattcgcaactcataatccgtagactacaagatgaaatctcaataaattatttttgagtttattaaagattgacattttaagtacaacttttaaggactaattactgtgatggacacagaaatgtagctgtgttctggaactgaatcttacatggtatacttagtgctgctgggtaatttgttggtatattatctggttagtggttaatgcttcctttaaaaataattgagtcatccattcactctttttcagttttatctgtcaatagtagctacatttttaatgggagcaccttttatcccaaagtgctttataaattgagtggactgatatatatcacacccaggtatcactgtgctgtcctttgctgtcagatttagaaatgtttttaagagctatgtgaaaacagacaatattagtttaggtcgggaactgagatattgtaatcaaatagttaacatcaggaagttaatttggctggcaaaattctagggaaacttggccagaaaactggtgttgaaggcttttgctcatataaacaagtgccattgagtttcaaatgaccagcaaatatatttagaactcaaaaaaaaaaaaaaaaa

SEQ ID NO: 26 - DNA, 2855 bp, Homo sapiens
  source 1..2855: mol_type = genomic DNA
aactttcatcccagcacatgatttgggaattacactttgtgacatggatgaatctgcactgacccttggtacaatagatgtttcttatctgccacattcatcagaatacagtgttggtcgatgtaagcacacaagtgaggaatggggtgagtgtggctttagacccaccgtcttcagatctgcaactttaaaatggaaagaaagcctaatgagtcggaaaaggccatttgttggaagatgttgttactcctgcactccccagagctgggacaaatttttcaaccccagtatcccgtctttgggtttgcggaatgttatttatatcaatgaaactcacacaagacaccgcggatggcttgcaagacgcctttcttacgttctttttattcaagagcgagatgtgcataagggcatgtttgccaccaatgtgactgaaaatgtgctgaacagcagtagagtacaagaggcaattgcagaagtggctgctgaattaaaccctgatggttctgcccagcagcaatcaaaagccgttaacaaagtgaaaaagaaagctaaaaggattcttcaagaaatggttgccactgtctcaccggcaatgatcagactgactgggtgggtgctgctaaaactgttcaacagcttcttttggaacattcaaattcacaaaggtcaacttgagatggttaaagctgcaactgagacgaatttgccgcttctgtttctaccagttcatagatcccatattgactatctgctgctcactttcattctcttctgccataacatcaaagcaccatacattgcttcaggcaataatctcaacatcccaatcttcagtaccttgatccataagcttgggggcttcttcatacgacgaaggctcgatgaaacaccagatggacggaaagatgttctctatagagctttgctccatgggcatatagttgaattacttcgacagcagcaattcttggagatcttcctggaaggcacacgttctaggagtggaaaaacctcttgtgctcgggcaggacttttgtcagttgtggtagatactctgtctaccaatgtcatcccagacatcttgataatacctgttggaatctcctatgatcgcattatcgaaggtcactacaatggtgaacaactgggcaaacctaagaagaatgagagcctgtggagtgtagcaagaggtgttactagaatgttacgaaaaaactatggttgtgtccgagtggattttgcacagccattttccttaaaggaatatttagaaagccaaagtcagaaaccggtgtctgctctactttccctggagcaagcgttgttaccagctatacttccttcaagacccagtgatgctgctgatgaaggtagagacacgtccattaatgagtccagaaatgcaacagatgaatccctacgaaggaggttgattgcaaatctggctgagcatattctattcactgctagcaagtcctgtgccattatgtccacacacattgtggcttgcctgctcctctacagacacaggcagggaattgatctctccacattggtcgaagacttctttgtgatgaaagaggaagtcctggctcgtgattttgacctggggttctcaggaaattcagaagatgtagtaatgcatgccatacagctgctgggaaattgtgtcacaatcacccacactagcaggaacgatgagttttttatcacccccagcacaactgtcccatcagtcttcgaactcaacttctacagcaatggggtacttcatgtctttatcatggaggccatcatagcttgcagcctttatgcagttctgaacaagaggggactggggggtcccactagcaccccacctaacctgatcagccaggagcagctggtgcggaaggcggccagcctgtgctaccttctctccaatgaaggcaccatctcactgccttgccagacattttaccaagtctgccatgaaacagtaggaaagtttatccagtatggcattcttacagtggcagagcacgatgaccaggaagatatcagtcctagtcttgctgagcagcagtgggacaagaagcttcctgaacctttgtcttggagaagtgatgaagaagatgaagacagtgactttggggaggaacagcgagattgctacctgaaggtgagccaatccaaggagcaccagcagtttatcaccttcttacagagactccttgggcctttgctggaggcctacagctctgctgccatctttgttcacaacttcagtggtcctgttccagaacctgagtatctgcaaaagttgcacaaatacctaataaccagaacagaaagaaatgttgcagtatatgctgagagtgccacatattgtcttgtgaagaatgctgtgaaaatgtttaaggatattggggttttcaaggagaccaaacaaaagagagtgtctgttttagaactgagcagcacttttctacctcaatgcaaccgacaaaaacttctagaatatattctgagttttgtggtgctgtaggtaacgtgtggcactgctggcaaatgaaggtcatgagatgagttccttgtaggtaccagcttctggctcaagagttgaaggtgccgtcgcagggtcaggcctgccctgtcccgaggtgatctcctggaagacaagtgccttctccctccatggatctgtgatcttcccagctctgcatcaacacagcagcctgcagataacacttggggggacctcagcctctattcgcaactcataatccgtagactacaagatgaaatctcaataaattatttttgagtttattaaagaggtcttttaaggcaaaaaaaaaaaaaaaaaaaaa

SEQ ID NO: 27 - protein, 828 aa, Homo sapiens
MDESALTLGTIDVSYLPHSSEYSVGRCKHTSEEWGECGFRPTIFRSATLKWKESLMSRKRPFVGRCCYSCTPQSWDKFFNPSIPSLGLRNVIYINETHTRHRGWLARRLSYVLFIQERDVHKGMFATNVTENVLNSSRVQEAIAEVAAELNPDGSAQQQSKAVNKVKKKAKRILQEMVATVSPAMIRLTGWVLLKLFNSFFWNIQIHKGQLEMVKAATETNLPLLFLPVHRSHIDYLLLTFILFCHNIKAPYIASGNNLNIPIFSTLIHKLGGFFIRRRLDETPDGRKDVLYRALLHGHIVELLRQQQFLEIFLEGTRSRSGKTSCARAGLLSVVVDTLSTNVIPDILIIPVGISYDRIIEGHYNGEQLGKPKKNESLWSVARGVIRMLRKNYGCVRVDFAQPFSLKEYLESQSQKPVSALLSLEQALLPAILPSRPSDAADEGRDTSINESRNATDESLRRRLIANLAEHILFTASKSCAIMSTHIVACLLLYRHRQGIDLSTLVEDFFVMKEEVLARDFDLGFSGNSEDVVMHAIQLLGNCVTITHTSRNDEFFITPSTTVPSVFELNFYSNGVLHVFIMEAIIACSLYAVLNKRGLGGPTSTPPNLISQEQLVRKAASLCYLLSNEGTISLPCQTFYQVCHETVGKFIQYGILTVAEHDDQEDISPSLAEQQWDKKLPEPLSWRSDEEDEDSDFGEEQRDCYLKVSQSKEHQQFITFLQRLLGPLLEAYSSAAIFVHNFSGPVPEPEYLQKLHKYLITRTERNVAVYAESATYCLVKNAVKMFKDIGVFKETKQKRVSVLELSSTFLPQCNRQKLLEYILSFVVL

SEQ ID NO: 28 - protein, 710 aa, Homo sapiens
MDESALTLGTIDVSYLPHSSEYSVGRCKHTSEEWGECGFRPTIFRSATLKWKESLMSRKRPFVGRCCYSCTPQSWDKFFNPSIPSLGLRNVIYINETHTRHRGWLARRLSYVLFIQERDVHKGMFATNVTENVLNSSRVQEAIAEVAAELNPDGSAQQQSKAVNKVKKKAKRILQEMVATVSPAMIRLTGWVLLKLFNSFFWNIQIHKGQLEMVKAATETNLPLLFLPVHRSHIDYLLLTFILFCHNIKAPYIASGNNLNIPIFSTLIHKLGGFFIRRRLDETPDGRKDVLYRALLHGHIVELLRQQQFLEIFLEGTRSRSGKTSCARAGLLSVVVDTLSTNVIPDILIIPVGISYDRIIEGHYNGEQLGKPKKNESLWSVARGVIRMLRKNYGCVRVDFAQPFSLKEYLESQSQKPVSALLSLEQALLPAILPSRPSDAADEGRDTSINESRNATDESLRRRLIANLAEHILFTASKSCAIMSTHIVACLLLYRHRQGIDLSTLVEDFFVMKEEVLARDFDLGFSGNSEDVVMHAIQLLGNCVTITHTSRNDEFFITPSTTVPSVFELNFYSNGVLHVFIMEAIIACSLYAVLNKRGLGGPTSTPPNLISQEQLVRKAASLCYLLSNEGTISLPCQTFYQVCHETVGKFIQYGILTVAEHDDQEDISPSLAEQQWDKKLPEPLSWRSDEEDEDSDFGEEQRDCYLKVLG

SEQ ID NO: 29 - protein, 828 aa, Homo sapiens [sequence omitted]

SEQ ID NO: 30 - protein, 710 aa, Homo sapiens
MDESALTLGTIDVSYLPHSSEYSVGRCKHTSEEWGECGFRPTVFRSATLKWKESLMSRKRPFVGRCCYSCTPQSWDKFFNPSIPSLGLRNVIYINETHTRHRGWLARRLSYVLFIQERDVHKGMFATNVTENVLNSSRVQEAIAEVAAELNPDGSAQQQSKAVNKVKKKAKRILQEMVATVSPAMIRLTGWVLLKLFNSFFWNIQIHKGQLEMVKAATETNLPLLFLPVHRSHIDYLLLTFILFCHNIKAPYIASGNNLNIPIFSTLIHKLGGFFIRRRLDETPDGRKDVLYRALLHGHIVELLRQQQFLEIFLEGTRSRSGKTSCARAGLLSVVVDTLSTNVIPDILIIPVGISYDRIIEGHYNGEQLGKPKKNESLWSVARGVIRMLRKNYGCVRVDFAQPFSLKEYLESQSQKPVSALLSLEQALLPAILPSRPSDAADEGRDTSINESRNATDESLRRRLIANLAEHILFTASKSCAIMSTHIVACLLLYRHRQGIDLSTLVEDFFVMKEEVLARDFDLGFSGNSEDVVMHAIQLLGNCVTITHTSRNDEFFITPSTTVPSVFELNFYSNGVLHVFIMEAIIACSLYAVLNKRGLGGPTSTPPNLISQEQLVRKAASLCYLLSNEGTISLPCQTFYQVCHETVGKFIQYGILTVAEHDDQEDISPSLAEQQWDKKLPEPLSWRSDEEDEDSDFGEEQRDCYLKVLG

SEQ ID NO: 31 - DNA, 20 bp, synthetic construct
  misc_feature 1..20: note = gRNA Recognition Sequence
  source 1..20: mol_type = other DNA
atggccttttccgactcatt

SEQ ID NO: 32 - DNA, 20 bp, synthetic construct
  misc_feature 1..20: note = gRNA Recognition Sequence
  source 1..20: mol_type = other DNA
aaagcctaatgagtcggaaa

SEQ ID NO: 33 - DNA, 20 bp, synthetic construct
  misc_feature 1..20: note = gRNA Recognition Sequence
  source 1..20: mol_type = other DNA
gtaaggttcttacccagctc

SEQ ID NO: 34 - DNA, 20 bp, synthetic construct
  misc_feature 1..20: note = gRNA Recognition Sequence
  source 1..20: mol_type = other DNA
attagggtcaataagcagta

SEQ ID NO: 35 - DNA, 20 bp, synthetic construct
  misc_feature 1..20: note = gRNA Recognition Sequence
  source 1..20: mol_type = other DNA
agtcggaaaaggccatttgt

SEQ ID NO: 36 - DNA, 20 bp, synthetic construct
  misc_feature 1..20: note = gRNA Recognition Sequence
  source 1..20: mol_type = other DNA
taaggttcttacccagctct

SEQ ID NO: 37 - DNA, 20 bp, synthetic construct
  misc_feature 1..20: note = gRNA Recognition Sequence
  source 1..20: mol_type = other DNA
aatatttgtcagggtgagtg

SEQ ID NO: 38 - DNA, 20 bp, synthetic construct
  misc_feature 1..20: note = gRNA Recognition Sequence
  source 1..20: mol_type = other DNA
gttgcagatctgaagatggt

SEQ ID NO: 39 - DNA, 20 bp, synthetic construct
  misc_feature 1..20: note = gRNA Recognition Sequence
  source 1..20: mol_type = other DNA
tacccagctctggggagtgc

SEQ ID NO: 40 - DNA, 20 bp, synthetic construct
  misc_feature 1..20: note = gRNA Recognition Sequence
  source 1..20: mol_type = other DNA
ggaaagaaagcctaatgagt

SEQ ID NO: 41 - DNA, 20 bp, synthetic construct
  misc_feature 1..20: note = gRNA Recognition Sequence
  source 1..20: mol_type = other DNA
aaggttcttacccagctctg

SEQ ID NO: 42 - DNA, 20 bp, synthetic construct
  misc_feature 1..20: note = gRNA Recognition Sequence
  source 1..20: mol_type = other DNA
agttgcagatctgaagatgg

SEQ ID NO: 43 - DNA, 20 bp, synthetic construct
  misc_feature 1..20: note = gRNA Recognition Sequence
  source 1..20: mol_type = other DNA
attatttccaactttgtagt

SEQ ID NO: 44 - DNA, 20 bp, synthetic construct
  misc_feature 1..20: note = gRNA Recognition Sequence
  source 1..20: mol_type = other DNA
taacaacatcttccaacaaa

(19) United States
(12) Patent Application Publication
(10) Pub. No.: US 2025/0197866 A1
(43) Pub. Date: Jun. 19, 2025

(54) Treatment Of Liver Disease With Mitochondrial Glycerol-3-Phosphate Acyltransferase (GPAM) Inhibitors

(71) Applicant: Regeneron Pharmaceuticals, Inc., Tarrytown, NY (US)

(72) Inventors: Luca Andrea Lotta, Tarrytown, NY (US); Niek Verweij, Tarrytown, NY (US); Colm O'Dushlaine, Tarrytown, NY (US); Jonathan Marchini, Tarrytown, NY (US); Aris Baras, Tarrytown, NY (US)

(21) Appl. No.: 19/000,435

(22) Filed: Dec. 23, 2024

Related U.S. Application Data
(62) Division of application No. 17/680,940, filed on Feb. 25, 2022, now Pat. No. 12,215,322.
(60) Provisional application No. 63/154,693, filed on Feb. 27, 2021.

(51) Int. Cl. C12N 15/113; A61K 31/7088; A61K 38/46; A61P 1/16; C12Q 1/6827; C12Q 1/6874; C12Q 1/6883

(52) U.S. Cl. CPC C12N 15/1137; A61K 31/7088; A61K 38/465; A61P 1/16; C12Q 1/6883; C12N 2310/14; C12N 2310/531; C12Q 1/6827; C12Q 1/6874; C12Q 2600/156; C12Q 2600/158

(57) ABSTRACT

The present disclosure provides methods of treating subjects having liver disease, and methods of identifying subjects having an increased risk of developing liver disease.

### REFERENCE TO SEQUENCE LISTING

This application includes a Sequence Listing submitted electronically as an XML file named 381204339SEQ, created on Dec. 11, 2024, with a size of 233,867 bytes. The Sequence Listing is incorporated herein by reference.

### FIELD

The present disclosure relates generally to the treatment of subjects having liver disease with Mitochondrial Glycerol-3-Phosphate Acyltransferase (GPAM) inhibitors, and methods of identifying subjects having an increased risk of developing liver disease.

### BACKGROUND

Chronic liver disease and cirrhosis are leading causes of morbidity and mortality in the United States accounting for 38,170 deaths (1.5% of total deaths) in 2014 (Kochanek et al., Nat'l. Vital Stat. Rep., 2016, 65, 1-122). The most common etiologies of cirrhosis in the U.S. are alcoholic liver disease, chronic hepatitis C, and nonalcoholic fatty liver disease (NAFLD), together accounting for about 80% of subjects awaiting liver transplant between 2004 and 2013 (Wong et al., Gastroenterology, 2015, 148, 547-555). The estimated prevalence of NAFLD in the U.S. is between 19 and 46 percent (Browning et al., Hepatology, 2004, 40, 1387-1395; Lazo et al., Am. J. Epidemiol., 2013, 178, 38-45; and Williams et al., Gastroenterology, 2011, 140, 124-131) and is rising over time (Younossi et al., Clin. Gastroenterol. Hepatol., 2011, 9, 524-530), likely in conjunction with increased rates of obesity, its primary risk factor (Cohen et al., Science, 2011, 332, 1519-1523). While significant advances have been made in the treatment of hepatitis C, there are currently no evidence-based treatments for alcoholic or nonalcoholic liver disease and cirrhosis.

Mitochondrial glycerol-3-phosphate acyltransferase (GPAM) is a member of protein family (pfam) 01553 family of glycerolipid acyltransferases located in the outer mitochondrial membrane. GPAM uses saturated fatty acids as its substrate for the synthesis of glycerolipids. GPAM esterifies the acyl-group from acyl-ACP to the sn-1 position of glycerol-3-phosphate, an essential step in glycerolipid biosynthesis, and the first essential step in triacylglycerols (TAG) synthesis. This metabolic pathway's first step is catalyzed by the encoded enzyme. GPAM is most highly expressed in liver and adipose tissue, but is also present in many other tissues including brain, kidney, heart, and adrenal gland.

### SUMMARY

The present disclosure provides methods of treating a subject having liver disease or having a risk of developing liver disease, the methods comprising administering a GPAM inhibitor to the subject.

The present disclosure also provides methods of treating a subject having fatty liver disease or liver fat or having a risk of developing fatty liver disease or liver fat, the methods comprising administering a GPAM inhibitor to the subject.

The present disclosure also provides methods of treating a subject having hepatocellular carcinoma or having a risk of developing hepatocellular carcinoma, the methods comprising administering a GPAM inhibitor to the subject.

The present disclosure also provides methods of treating a subject having liver cirrhosis or having a risk of developing liver cirrhosis, the methods comprising administering a GPAM inhibitor to the subject.

The present disclosure also provides methods of treating a subject having liver fibrosis or having a risk of developing liver fibrosis, the methods comprising administering a GPAM inhibitor to the subject.

The present disclosure also provides methods of treating a subject having simple steatosis, steatohepatitis, or non-alcoholic steatohepatitis (NASH) or having a risk of developing simple steatosis, steatohepatitis, or NASH, the methods comprising administering a GPAM inhibitor to the subject.

The present disclosure also provides methods of treating a subject with a therapeutic agent that treats or inhibits liver disease, wherein the subject is suffering from liver disease, the methods comprising the steps of: determining whether the subject has a GPAM predicted loss-of-function variant nucleic acid molecule encoding a human GPAM polypeptide by: obtaining or having obtained a biological sample from the subject; and performing or having performed a sequence analysis on the biological sample to determine if the subject has a genotype comprising the GPAM predicted loss-of-function variant nucleic acid molecule; and when the subject is GPAM reference, then administering or continuing to administer to the subject the therapeutic agent that treats or inhibits liver disease in a standard dosage amount, and administering to the subject a GPAM inhibitor; and when the subject is heterozygous for a GPAM predicted loss-of-function variant, then administering or continuing to administer to the subject the therapeutic agent that treats or inhibits liver disease in an amount that is the same as or lower than a standard dosage amount, and administering to the subject a GPAM inhibitor; wherein the presence of a genotype having the GPAM predicted loss-of-function variant nucleic acid molecule encoding the human GPAM polypeptide indicates the subject has a reduced risk of developing liver disease.

The present disclosure also provides methods of identifying a subject having an increased risk for developing liver disease, wherein the methods comprise: determining or having determined the presence or absence of a glycerol-3-phosphate acyltransferase (GPAM) predicted loss-of-function variant nucleic acid molecule encoding a human GPAM polypeptide in a biological sample obtained from the subject; wherein: when the subject is GPAM reference, then the subject has an increased risk for developing liver disease; and when the subject is heterozygous or homozygous for a GPAM predicted loss-of-function variant, then the subject has a decreased risk for developing liver disease.

The present disclosure also provides therapeutic agents that treat or inhibit liver disease for use in the treatment of liver disease in a subject having: a genomic nucleic acid molecule having a nucleotide sequence encoding a human GPAM polypeptide, wherein the nucleotide sequence comprises a guanine at a position corresponding to position 3,195 according to SEQ ID NO:2, or the complement thereof; an mRNA molecule having a nucleotide sequence encoding a human GPAM polypeptide, wherein the nucleotide sequence comprises a guanine at a position corresponding to: position 327 according to SEQ ID NO:9, or the complement thereof; position 291 according to SEQ ID NO:10, or the complement thereof; position 323 according to SEQ ID NO:11, or the complement thereof; position 326 according to SEQ ID NO:12, or the complement thereof; position 305 according to SEQ ID NO:13, or the complement thereof; or position 170 according to SEQ ID NO:14, or the complement thereof; or a cDNA molecule having a nucleotide sequence encoding a human GPAM polypeptide, wherein the nucleotide sequence comprises a guanine at a position corresponding to: position 327 according to SEQ ID NO:21, or the complement thereof; position 291 according to SEQ ID NO:22, or the complement thereof; position 323 according to SEQ ID NO:23, or the complement thereof; position 326 according to SEQ ID NO:24, or the complement thereof; position 305 according to SEQ ID NO:25, or the complement thereof; or position 170 according to SEQ ID NO:26, or the complement thereof.

The present disclosure also provides GPAM inhibitors for use in the treatment of liver disease in a subject having: a genomic nucleic acid molecule having a nucleotide sequence encoding a human GPAM polypeptide, wherein the nucleotide sequence comprises a guanine at a position corresponding to position 3,195 according to SEQ ID NO:2, or the complement thereof; an mRNA molecule having a nucleotide sequence encoding a human GPAM polypeptide, wherein the nucleotide sequence comprises a guanine at a position corresponding to: position 327 according to SEQ ID NO:9, or the complement thereof; position 291 according to SEQ ID NO:10, or the complement thereof; position 323 according to SEQ ID NO:11, or the complement thereof; position 326 according to SEQ ID NO:12, or the complement thereof; position 305 according to SEQ ID NO:13, or the complement thereof; or position 170 according to SEQ ID NO:14, or the complement thereof; or a cDNA molecule having a nucleotide sequence encoding a human GPAM polypeptide, wherein the nucleotide sequence comprises a guanine at a position corresponding to: position 327 according to SEQ ID NO:21, or the complement thereof; position 291 according to SEQ ID NO:22, or the complement thereof; position 323 according to SEQ ID NO:23, or the complement thereof; position 326 according to SEQ ID NO:24, or the complement thereof; position 305 according to SEQ ID NO:25, or the complement thereof; or position 170 according to SEQ ID NO:26, or the complement thereof.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

A common missense variant in the GPAM gene associated with a decreased risk of developing liver disease in human subjects has been identified in accordance with the present disclosure. For example, a genetic alteration that changes the adenine nucleotide of position 3,195 in the human GPAM reference (see, SEQ ID NO:1) to guanine has been observed to indicate that the human having such an alteration may have a decreased risk of developing liver disease. Altogether, the genetic analyses described herein surprisingly indicate that the GPAM gene and, in particular, a variant in the GPAM gene, associates with a decreased risk of developing liver disease. Therefore, subjects that are GPAM reference that have an increased risk of developing liver disease (such as, for example, fatty liver disease (including alcoholic fatty liver disease (AFLD) and NAFLD), hepatocellular carcinoma, liver cirrhosis, liver fibrosis, simple steatosis, steatohepatitis, non-alcoholic steatohepatitis (NASH), and parenchymal liver disease) may be treated such that liver disease is prevented, the symptoms thereof are reduced, and/or development of symptoms is repressed. Accordingly, the present disclosure provides methods of leveraging the identification of such variants in subjects to identify or stratify risk in such subjects of developing liver disease, or to diagnose subjects as having an increased risk of developing liver disease, such that subjects at risk or subjects with active disease may be treated accordingly.

It has been further observed in accordance with the present disclosure that an aggregate burden of certain GPAM variants associate with a lower risk of developing liver disease (such as, for example, fatty liver disease (including AFLD and NAFLD), hepatocellular carcinoma, liver cirrhosis, liver fibrosis, simple steatosis, steatohepatitis, non-alcoholic steatohepatitis (NASH), and parenchymal liver disease). Therefore, it is believed that humans having liver disease may be treated with molecules that inhibit GPAM. Accordingly, the present disclosure provides methods for leveraging the identification of such variants, and an aggregation burden of having such variants, in subjects to identify or stratify risk in such subjects of developing liver disease, or to diagnose subjects as having liver disease, such that subjects at risk or subjects with active disease may be treated.

For purposes of the present disclosure, any particular human can be categorized as having one of three GPAM genotypes: i) GPAM reference; ii) heterozygous for a GPAM predicted loss-of-function variant; or iii) homozygous for a GPAM predicted loss-of-function variant. A human is GPAM reference when the human does not have a copy of a GPAM predicted loss-of-function variant nucleic acid molecule. A human is heterozygous for a GPAM predicted loss-of-function variant when the human has a single copy of a GPAM predicted loss-of-function variant nucleic acid molecule. A GPAM predicted loss-of-function variant nucleic acid molecule is any GPAM nucleic acid molecule (such as, a genomic nucleic acid molecule, an mRNA molecule, or a cDNA molecule) encoding a GPAM polypeptide having a partial loss-of-function, a complete loss-of-function, a predicted partial loss-of-function, or a predicted complete loss-of-function. A human who has a GPAM polypeptide having a partial loss-of-function (or predicted partial loss-of-function) is hypomorphic for GPAM. The GPAM predicted loss-of-function variant nucleic acid molecule can be any nucleic acid molecule encoding GPAM Ile43Val. GPAM Ile43Val is believed to be at least a partial predicted loss-of-function variant. A human is homozygous for a GPAM predicted loss-of-function variant when the human has two copies of a GPAM predicted loss-of-function variant nucleic acid molecule.

For subjects that are genotyped or determined to be GPAM reference, such subjects have an increased risk of developing liver disease (such as, for example, fatty liver disease (including AFLD and NAFLD), hepatocellular carcinoma, liver cirrhosis, liver fibrosis, simple steatosis, steatohepatitis, non-alcoholic steatohepatitis (NASH), and parenchymal liver disease). For subjects that are genotyped or determined to be either GPAM reference or heterozygous for a GPAM predicted loss-of-function variant, such subjects can be treated with a GPAM inhibitor.

In any of the embodiments described herein, the GPAM predicted loss-of-function variant nucleic acid molecule can be any GPAM nucleic acid molecule (such as, for example, genomic nucleic acid molecule, mRNA molecule, or cDNA molecule) encoding a GPAM polypeptide having a partial loss-of-function, a complete loss-of-function, a predicted partial loss-of-function, or a predicted complete loss-of-function. For example, the GPAM predicted loss-of-function variant nucleic acid molecule can be any nucleic acid molecule encoding GPAM Ile43Val. In any of the embodiments described herein, the GPAM variant nucleic acid molecule can be any GPAM nucleic acid molecule that is a missense variant nucleic acid molecule.

In any of the embodiments described herein, the GPAM predicted loss-of-function polypeptide can be any GPAM polypeptide having a partial loss-of-function, a complete loss-of-function, a predicted partial loss-of-function, or a predicted complete loss-of-function. In any of the embodiments described herein, the GPAM predicted loss-of-function polypeptide can be any of the GPAM polypeptides described herein including, for example, GPAM Ile43Val.

In any of the embodiments described herein, the liver disease is a fatty liver disease, including AFLD and NAFLD, hepatocellular carcinoma, liver cirrhosis, liver fibrosis, simple steatosis, steatohepatitis, NASH, or parenchymal liver disease. In some embodiments, the liver disease is a fatty liver disease. In some embodiments, the liver disease is AFLD. In some embodiments, the liver disease is NAFLD. In some embodiments, the liver disease is hepatocellular carcinoma. In some embodiments, the liver disease is liver cirrhosis. In some embodiments, the liver disease is liver fibrosis. In some embodiments, the liver disease is simple steatosis. In some embodiments, the liver disease is steatohepatitis. In some embodiments, the liver disease is NASH. In some embodiments, the liver disease is parenchymal liver disease.

In some embodiments, the liver disease is liver damage, deposition of liver fat, liver inflammation, toxic liver disease, immune liver disease, or elevated alanine aminotransferase (ALT). In some embodiments, the liver disease is liver damage. In some embodiments, the liver damage is measured by elevation of liver enzymes. In some embodiments, the liver disease is deposition of liver fat. In some embodiments, the deposition of liver fat is identified by imaging, biopsy, or other procedure. In some embodiments, the liver disease is liver inflammation. In some embodiments, the liver inflammation is identified by biopsy, imaging, or other procedure. In some embodiments, the liver disease is toxic liver disease. In some embodiments, the liver disease is immune liver disease. In some embodiments, the liver disease is elevated ALT. In some embodiments, the liver disease is due to accumulation of metals, proteinaceous material, bile acids, or other irritant or pro-inflammatory materials. In some embodiments, the liver disease is due to accumulation of metals, such as iron. In some embodiments, the liver disease is due to accumulation of proteinaceous material, such as in alpha 1 antitrypsin deficiency. In some embodiments, the liver disease is due to accumulation of bile acids. In some embodiments, the liver disease is due to accumulation of an irritant. In some embodiments, the liver disease is due to accumulation of a pro-inflammatory material.

Symptoms of liver disease include, but are not limited to, enlarged liver, fatigue, pain in the upper right abdomen, abdominal swelling (ascites), enlarged blood vessels just beneath the skin's surface, enlarged breasts in men, enlarged spleen, red palms, and yellowing of the skin and eyes (jaundice), pruritus, dark urine color, pale stool color nausea or vomiting, loss of appetite, and tendency to bruise easily. Testing for liver diseases can involve blood tests, imaging of the liver, and biopsy of the liver. An individual is at increased risk of a liver disease if the subject has at least one known risk-factor (e.g., genetic factor such as a disease-causing mutation) placing individuals with that risk factor at a statistically significant greater risk of developing the disease than individuals without the risk factor. Risk factors for liver diseases are also well known and can include, for example, excessive alcohol use, obesity, high cholesterol, high levels of triglycerides in the blood, polycystic ovary syndrome, sleep apnea, type 2 diabetes, underactive thyroid (hypothyroidism), underactive pituitary gland (hypopituitarism), and metabolic syndromes including raised blood lipids.

The present disclosure provides methods of treating a subject having liver disease, the methods comprising administering a GPAM inhibitor to the subject.

The present disclosure also provides methods of treating a subject having fatty liver disease (such as AFLD or NAFLD), the methods comprising administering a GPAM inhibitor to the subject.

The present disclosure also provides methods of treating a subject having hepatocellular carcinoma, the methods comprising administering a GPAM inhibitor to the subject.

The present disclosure also provides methods of treating a subject having liver cirrhosis, the methods comprising administering a GPAM inhibitor to the subject.

The present disclosure also provides methods of treating a subject having liver fibrosis, the methods comprising administering a GPAM inhibitor to the subject.

The present disclosure also provides methods of treating a subject having simple steatosis, steatohepatitis, or NASH, the methods comprising administering a GPAM inhibitor to the subject.

In some embodiments, the GPAM inhibitor comprises an inhibitory nucleic acid molecule. In some embodiments, the inhibitory nucleic acid molecule comprises an antisense molecule. In some embodiments, the inhibitory nucleic acid molecule comprises a small interfering RNA (siRNA) molecule. In some embodiments, the inhibitory nucleic acid molecule comprises a short hairpin RNA (shRNA) molecule. Such inhibitory nucleic acid molecules can be designed to target any region of a GPAM mRNA. In some embodiments, the inhibitory nucleic acid molecule hybridizes to a sequence within a GPAM genomic nucleic acid molecule or mRNA molecule and decreases expression of the GPAM polypeptide in a cell in the subject. In some embodiments, the GPAM inhibitor comprises an antisense RNA that hybridizes to a GPAM genomic nucleic acid molecule or mRNA molecule and decreases expression of the GPAM polypeptide in a cell in the subject. In some embodiments, the GPAM inhibitor comprises an siRNA that hybridizes to a GPAM genomic nucleic acid molecule or mRNA molecule and decreases expression of the GPAM polypeptide in a cell in the subject. In some embodiments, the GPAM inhibitor comprises an shRNA that hybridizes to a GPAM genomic nucleic acid molecule or mRNA molecule and decreases expression of the GPAM polypeptide in a cell in the subject. In some embodiments, the inhibitory nucleic acid molecule is not an siRNA molecule.

In some embodiments, the GPAM inhibitor comprises a nuclease agent that induces one or more nicks or double-strand breaks at a recognition sequence(s) or a DNA-binding protein that binds to a recognition sequence within a GPAM genomic nucleic acid molecule. The recognition sequence can be located within a coding region of the GPAM gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the GPAM gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify a GPAM genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of GPAM nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a GPAM genomic nucleic acid molecule or it can be a nickase that creates a single-strand break in a GPAM genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of GPAM genomic nucleic acid molecules can be generated by contacting a cell with a Cas protein and one or more gRNAs that hybridize to one or more gRNA recognition sequences within a target genomic locus in the GPAM genomic nucleic acid molecule. For example, a gRNA recognition sequence can be located within a region of SEQ ID NO:1. The gRNA recognition sequence can also include or be proximate to a position corresponding to: position 3,195 according to SEQ ID NO:1. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to: position 3,195 according to SEQ ID NO:1. The gRNA recognition sequence can include or be proximate to the start codon of a GPAM genomic nucleic acid molecule or the stop codon of a GPAM genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a GPAM genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a GPAM genomic nucleic acid molecule. An exemplary gRNA is a gRNA effective to direct a Cas enzyme to bind to or cleave a GPAM genomic nucleic acid molecule, wherein the gRNA comprises a DNA-targeting segment that hybridizes to a gRNA recognition sequence within the GPAM genomic nucleic acid molecule that includes or is proximate to a position corresponding to: position 3,195 according to SEQ ID NO:1. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of a position corresponding to: position 3,195 according to SEQ ID NO:1. Other exemplary gRNAs comprise a DNA-targeting segment that hybridizes to a gRNA recognition sequence present within a GPAM genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the human GPAM reference gene are set forth in Table 1 as SEQ ID NOs:31-44.

TABLE 1
Guide RNA Recognition Sequences
Near GPAM Variation

| | gRNA Recognition | SEQ ID |
|---|---|---|
| Strand | Sequence | NO: |
| − | ATGGCCTTTTCCGACTCATT | 31 |
| + | AAAGCCTAATGAGTCGGAAA | 32 |
| − | GTAAGGTTCTTACCCAGCTC | 33 |
| − | ATTAGGGTCAATAAGCAGTA | 34 |
| + | AGTCGGAAAAGGCCATTTGT | 35 |
| − | TAAGGTTCTTACCCAGCTCT | 36 |
| + | AATATTTGTCAGGGTGAGTG | 37 |
| − | GTTGCAGATCTGAAGATGGT | 38 |
| − | TACCCAGCTCTGGGGAGTGC | 39 |
| + | GGAAAGAAAGCCTAATGAGT | 40 |
| − | AAGGTTCTTACCCAGCTCTG | 41 |
| − | AGTTGCAGATCTGAAGATGG | 42 |
| + | ATTATTTCCAACTTTGTAGT | 43 |
| − | TAACAACATCTTCCAACAAA | 44 |

The Cas protein and the gRNA form a complex, and the Cas protein cleaves the target GPAM genomic nucleic acid molecule. The Cas protein can cleave the nucleic acid molecule at a site within or outside of the nucleic acid sequence present in the target GPAM genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the GPAM genomic nucleic acid molecule to which a DNA-targeting segment of a gRNA will bind.

Such methods can result, for example, in a GPAM genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the GPAM genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the GPAM inhibitor comprises a small molecule. In some embodiments, the GPAM inhibitor is FSG67. In some embodiments, the GPAM inhibitor comprises: substituted or unsubstituted benzoic acid derivatives (such as, for example, 2-(alkanesulfonamido)benzoic acid, such as 4-([1,1′-biphenyl]-4-carbonyl)-2-(octane sulfonamido)benzoic acid)) (see, Outlaw et al., Med. Chem. Comm., 2014, 5, 826-830); substituted or unsubstituted 7-aminoindole derivatives (such as, for example, 7-amino-5-cyanoindoles) (see, Outlaw et al., Org. Lett., 2014, 16, 6334-6337); substituted or unsubstituted benzoic acid derivatives (such as, for example, 2-(nonylsulfonamido)benzoic acid) (see, Wydysh et al., J. Med. Chem., 2011, 52, 3317-3327); substituted or unsubstituted thiophenes and thiolactones, substituted or unsubstituted phosphonates, substituted or unsubstituted phenyls, substituted or unsubstituted benzoic acids, substituted or unsubstituted 2-, 3-, or 4-(alkanesulfonamido)benzoic acids, substituted or unsubstituted 2-, 3-, or 4-(alkanesulfonamido)benzoic acids, or 2-, 3-, or 4-(alkanesulfonamido)benzoic phosphonic acids (see, U.S. Pat. No. 9,149,445); and substituted or unsubstituted 2-(alkanesulfonamido)benzoic acids (see, PCT Publication No. WO 2019/165232).

In some embodiments, the methods of treatment further comprise detecting the presence or absence of a GPAM predicted loss-of-function variant nucleic acid molecule encoding a human GPAM polypeptide in a biological sample from the subject. As used throughout the present disclosure, a “GPAM predicted loss-of-function variant nucleic acid molecule” is any GPAM nucleic acid molecule (such as, for example, genomic nucleic acid molecule, mRNA molecule, or cDNA molecule) encoding a GPAM polypeptide having a partial loss-of-function, a complete loss-of-function, a predicted partial loss-of-function, or a predicted complete loss-of-function.

The present disclosure also provides methods of treating a subject with a therapeutic agent that treats or inhibits liver disease, wherein the subject is suffering from liver disease. In some embodiments, the methods comprise determining whether the subject has a GPAM predicted loss-of-function variant nucleic acid molecule encoding a human GPAM polypeptide by obtaining or having obtained a biological sample from the subject, and performing or having performed a sequence analysis on the biological sample to determine if the subject has a genotype comprising the GPAM predicted loss-of-function variant nucleic acid molecule. When the subject is GPAM reference, the therapeutic agent that treats or inhibits liver disease is administered or continued to be administered to the subject in a standard dosage amount, and a GPAM inhibitor is administered to the subject. When the subject is heterozygous for a GPAM predicted loss-of-function variant, the therapeutic agent that treats or inhibits liver disease is administered or continued to be administered to the subject in an amount that is the same as or lower than a standard dosage amount, and a GPAM inhibitor is administered to the subject. The presence of a genotype having the GPAM predicted loss-of-function variant nucleic acid molecule encoding the human GPAM polypeptide indicates the subject has a reduced risk of developing liver disease. In some embodiments, the subject is GPAM reference. In some embodiments, the subject is heterozygous for a GPAM predicted loss-of-function variant.

In some embodiments, the methods comprise determining the subject's aggregate burden of having a plurality of GPAM predicted loss-of-function variant genomic nucleic acid molecules, GPAM predicted loss-of-function variant mRNA molecules, and/or GPAM predicted loss-of-function variant cDNA molecules produced from the mRNA molecules, by: performing or having performed a sequence analysis on a biological sample obtained from the subject to determine the subject's aggregate burden. When the subject has a lower aggregate burden, the subject is at a higher risk of developing a liver disease and the subject is administered or continued to be administered the therapeutic agent that treats or inhibits liver disease in a standard dosage amount. When the subject has a greater aggregate burden, the subject is at a lower risk of developing a liver disease and the subject is administered or continued to be administered the therapeutic agent that treats or inhibits liver disease in an amount that is the same as or lower than the standard dosage amount. The greater the aggregate burden, the lower the risk of developing liver disease.

For subjects that are genotyped or determined to be either GPAM reference or heterozygous for a GPAM predicted loss-of-function variant, such subjects can be treated with a GPAM inhibitor, as described herein.

Detecting the presence or absence of a GPAM predicted loss-of-function variant nucleic acid molecule in a biological sample from a subject and/or determining whether a subject has a GPAM predicted loss-of-function variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is GPAM reference, the subject is also administered a therapeutic agent that treats or inhibits liver disease in a standard dosage amount. In some embodiments, when the subject is heterozygous for a GPAM predicted loss-of-function variant, the subject is also administered a therapeutic agent that treats or inhibits liver disease in a dosage amount that is the same as or lower than a standard dosage amount.

In some embodiments, the treatment methods further comprise detecting the presence or absence of a GPAM predicted loss-of-function polypeptide in a biological sample from the subject. In some embodiments, when the subject does not have a GPAM predicted loss-of-function polypeptide, the subject is also administered a therapeutic agent that treats or inhibits liver disease in a standard dosage amount. In some embodiments, when the subject has a GPAM predicted loss-of-function polypeptide, the subject is also administered a therapeutic agent that treats or inhibits liver disease in a dosage amount that is the same as or lower than a standard dosage amount.

The present disclosure also provides methods of treating a subject with a therapeutic agent that treats or inhibits liver disease, wherein the subject is suffering from liver disease. In some embodiments, the method comprises determining whether the subject has a GPAM predicted loss-of-function polypeptide by obtaining or having obtained a biological sample from the subject, and performing or having performed an assay on the biological sample to determine if the subject has a GPAM predicted loss-of-function polypeptide. When the subject does not have a GPAM predicted loss-of-function polypeptide, the therapeutic agent that treats or inhibits liver disease is administered or continued to be administered to the subject in a standard dosage amount, and a GPAM inhibitor is administered to the subject. When the subject has a GPAM predicted loss-of-function polypeptide, the therapeutic agent that treats or inhibits liver disease is administered or continued to be administered to the subject in an amount that is the same as or lower than a standard dosage amount, and a GPAM inhibitor is administered to the subject. The presence of a GPAM predicted loss-of-function polypeptide indicates the subject has a reduced risk of developing liver disease. In some embodiments, the subject has a GPAM predicted loss-of-function polypeptide. In some embodiments, the subject does not have a GPAM predicted loss-of-function polypeptide.

Detecting the presence or absence of a GPAM predicted loss-of-function polypeptide in a biological sample from a subject and/or determining whether a subject has a GPAM predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

Examples of therapeutic agents that treat or inhibit liver disease include, but are not limited to: Disulfiram, Naltrexone, Acamprosate, Prednisone, Prednisone, Azathioprine, Penicillamine, Trientine, Deferoxamine, Ciprofloxacin, Norofloxacin, Ceftriaxone, Ofloxacin, Amoxicillin-clavulanate, Phytonadione, Bumetanide, Furosemide, Hydrochlorothiazide, Chlorothiazide, Amiloride, Triamterene, Spironolactone, Octreotide, Atenolol, Metoprolol, Nadolol, Propranolol, Timolol, and Carvedilol.

Additional examples of liver disease therapeutic agents (e.g., for use in chronic hepatitis C treatment) include, but are not limited to, ribavirin, paritaprevir, simeprevir (Olysio), grazoprevir, ledipasvir, ombitasvir, elbasvir, daclatasvir (Daklinza), dasabuvir, ritonavir, sofosbuvir, velpatasvir, voxilaprevir, glecaprevir, pibrentasvir, peginterferon alfa-2a, peginterferon alfa-2b, and interferon alfa-2b.

Additional examples of liver disease therapeutic agents (e.g., for use in nonalcoholic fatty liver disease) include, but are not limited to, weight loss inducing agents such as orlistat or sibutramine; insulin sensitizing agents such as thiazolidinediones (TZDs), metformin, and meglitinides; lipid lowering agents such as statins, fibrates, and omega-3 fatty acids; antioxidants such as, vitamin E, betaine, N-Acetyl-cysteine, lecithin, silymarin, and beta-carotene; anti TNF agents such as pentoxifylline; probiotics, such as VSL #3; and cytoprotective agents such as ursodeoxycholic acid (UDCA). Other suitable treatments include ACE inhibitors/ARBs, oligofructose, and Incretin analogs.

Additional examples of liver disease therapeutic agents (e.g., for use in NASH) include, but are not limited to, OCALIVA® (obeticholic acid), Selonsertib, Elafibranor, Cenicriviroc, GR_MD_02, MGL_3196, IMM124E, arachidyl amido cholanoic acid (ARAMCHOL™), GS0976, Emricasan, Volixibat, NGM282, GS9674, Tropifexor, MN_001, LMB763, BI_1467335, MSDC_0602, PF_05221304, DF102, Saroglitazar, BMS986036, Lanifibranor, Semaglutide, Nitazoxanide, GRI_0621, EYP001, VK2809, Nalmefene, LIK066, MT_3995, Elobixibat, Namodenoson, Foralumab, SAR425899, Sotagliflozin, EDP_305, Isosabutate, Gemcabene, TERN_101, KBP_042, PF_06865571, DUR928, PF_06835919, NGM313, BMS_986171, Namacizumab, CER_209, ND_L02_s0201, RTU_1096, DRX_065, IONIS_DGAT2Rx, INT_767, NC_001, Seladepar, PXL770, TERN_201, NV556, AZD2693, SP_1373, VK0214, Hepastem, TGFTX4, RLBN1127, GKT_137831, RYI_018, CB4209-CB4211, and JH_0920.

In some embodiments, the dose of the therapeutic agents that treat or inhibit liver disease can be reduced by about 10%, by about 20%, by about 30%, by about 40%, by about 50%, by about 60%, by about 70%, by about 80%, or by about 90% for subjects that are heterozygous for a GPAM predicted loss-of-function variant (i.e., a lower than the standard dosage amount) compared to subjects that are GPAM reference (who may receive a standard dosage amount). In some embodiments, the dose of the therapeutic agents that treat or inhibit liver disease can be reduced by about 10%, by about 20%, by about 30%, by about 40%, or by about 50%. In addition, the dose of therapeutic agents that treat or inhibit liver disease in subjects that are heterozygous for a GPAM predicted loss-of-function variant can be administered less frequently compared to subjects that are GPAM reference.

Administration of the therapeutic agents that treat or inhibit liver disease and/or GPAM inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat or inhibit liver disease and/or GPAM inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

In any of the embodiments described herein, any of the therapeutic compounds described herein can be formulated into pharmaceutical compositions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, the pharmaceutical compositions can comprise: a) a safe and therapeutically effective amount of a compound described herein, or a pharmaceutically acceptable salt thereof, and/or b) a pharmaceutically acceptable carrier, diluent, excipient, or combination thereof.

In any of the embodiments described herein, any of the therapeutic compounds described herein can be formulated into a single pharmaceutical composition. In some embodiments, any of the therapeutic compounds described herein can be administered in combination with one or more second pharmaceutical agents or a pharmaceutical composition comprising one or more second pharmaceutical agents.

The term “pharmaceutically acceptable carrier” or “pharmaceutically acceptable excipient” includes any and all solvents, diluents, emulsifiers, binders, buffers, dispersion media, coatings, antibacterial and antifungal agents, isotonic and absorption delaying agents and the like, or any other such compound useful in preparing pharmaceutical formulations. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

In some embodiments, substances which can serve as pharmaceutically-acceptable carriers or components thereof include, but are not limited to: sugars, such as lactose, glucose, and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil, and oil of theobroma; polyols such as propylene glycol, glycerine, sorbitol, mannitol, and polyethylene glycol; alginic acid; emulsifiers, such as the TWEENS; wetting agents, such as sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the subject compound can be determined depending on the manner the compound is to be administered.

The pharmaceutical compositions described herein can be provided in unit dosage form. As used herein, a “unit dosage form” is a composition containing an amount of a compound that is suitable for administration to a subject, in a single dose, according to good medical practice. The preparation of a single or unit dosage form, however, does not imply that the dosage form is administered once per day or once per course of therapy. A unit dosage form may comprise a single daily dose or a fractional sub-dose wherein several unit dosage forms are to be administered over the course of a day to complete a daily dose. In some embodiments, a unit dosage form may be administered more or less often than once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the compositions administered according to the methods described herein may also be administered as a continuous infusion or via an implantable infusion pump.

In some embodiments, various oral dosage forms can be used, including solid forms such as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid, microcrystalline cellulose, carboxymethyl cellulose, and talc. Tablets may also comprise solubilizers or emulsifiers, such as poloxamers, cremophor/Kolliphor®/Lutrol®, methylcellulose, hydroxypropylmethylcellulose, or others as are known in the art. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, as desired.

Peroral (PO) compositions also include liquid solutions, emulsions, suspensions, and the like. Typical components of carriers for syrups, elixirs, emulsions and suspensions include ethanol, glycerol, propylene glycol, polyethylene glycol, liquid sucrose, sorbitol, and water. For a suspension, typical suspending agents include methyl cellulose, sodium carboxymethyl cellulose, AVICEL RC-591, tragacanth, and sodium alginate; typical wetting agents include lecithin and polysorbate 80; and typical preservatives include methyl paraben and sodium benzoate. Peroral liquid compositions may also contain one or more components such as sweeteners, flavoring agents, and colorants.

In some embodiments, the compositions may also be coated by conventional methods, typically with pH or time-dependent coatings, such that the compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes, and shellac.

In some embodiments, compositions useful for attaining systemic delivery of the subject compounds include sublingual, buccal and nasal dosage forms. Such compositions typically comprise one or more of soluble filler substances such as sucrose, sorbitol, and mannitol; and binders such as acacia, microcrystalline cellulose, carboxymethyl cellulose, and hydroxypropyl methyl cellulose. Glidants, lubricants, sweeteners, colorants, antioxidants, and flavoring agents can also be included.

In some embodiments, preservatives that can be used in the pharmaceutical compositions disclosed herein include, but are not limited to, benzalkonium chloride, PHMB, chlorobutanol, thimerosal, phenylmercuric, acetate, and phenylmercuric nitrate. A useful surfactant is, for example, Tween 80. Likewise, various useful vehicles including, but are not limited to, polyvinyl alcohol, povidone, hydroxypropyl methyl cellulose, poloxamers, carboxymethyl cellulose, hydroxyethyl cellulose, and purified water can be used.

In some embodiments, tonicity adjustors can be added as needed or convenient. They include, but are not limited to, salts, particularly sodium chloride, potassium chloride, mannitol, and glycerin, or any other suitable tonicity adjustor.

In some embodiments, various buffers and means for adjusting pH can be used. In some embodiments, the pH will be between 4 and 9. Suitable buffers include, but are not limited to, acetate buffers, citrate buffers, phosphate buffers, and borate buffers. Acids or bases may be used to adjust the pH of these formulations as needed.

In some embodiments, the compositions can comprise antioxidants including, but not limited to sodium metabi sulfite, sodium thiosulfate, acetylcysteine, butylated hydroxyanisole, and butylated hydroxytoluene.

In some embodiments, the compositions can comprise other excipient components such as chelating agents. A useful chelating agent is edetate disodium, although other chelating agents can also be used.

In some embodiments, the composition is for topical use, including for transdermal administration, creams, ointments, gels, solutions or suspensions, etc. Topical formulations can generally be comprised of a pharmaceutical carrier, co-solvent, emulsifier, penetration enhancer, preservative system, and emollient.

For intravenous administration, the compounds and compositions described herein can be dissolved or dispersed in a pharmaceutically acceptable diluent, such as a saline or dextrose solution. Suitable excipients can be included to achieve the desired pH, including but not limited to, NaOH, sodium carbonate, sodium acetate, HCl, and citric acid. In some embodiments, the pH of the final composition ranges from 2 to 8 or from 4 to 7. Antioxidant excipients can include sodium bisulfite, acetone sodium bisulfite, sodium formaldehyde, sulfoxylate, thiourea, and EDTA. Other non-limiting examples of suitable excipients found in the final intravenous composition can include sodium or potassium phosphates, citric acid, tartaric acid, gelatin, and carbohydrates such as dextrose, mannitol, and dextran. Additional acceptable excipients are described in Powell et al., PDA J. Pharm. Sci. and Tech., 1998, 52 238-311 and Nema et al., PDA J. Pharm. Sci. Tech., 2011, 65 287-332. Antimicrobial agents can also be included to achieve a bacteriostatic or fungistatic solution, including but not limited to, phenylmercuric nitrate, thimerosal, benzethonium chloride, benzalkonium chloride, phenol, cresol, and chlorobutanol.

In some embodiments, the compositions for intravenous administration can be provided to caregivers in the form of one more solids that are reconstituted with a suitable diluent such as sterile water, saline or dextrose in water shortly prior to administration. In some embodiments, the compositions are provided in solution ready to administer parenterally. In some embodiments, the compositions are provided in a solution that is further diluted prior to administration. In embodiments that include administering a combination of a compound described herein and another agent, the combination can be provided to caregivers as a mixture, or the caregivers can mix the two agents prior to administration, or the two agents can be administered separately.

The actual unit dose of the compounds described herein and/or second pharmaceutical agents described herein depends on the specific compound, and on the condition to be treated. In some embodiments, the dose can be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight. In some embodiments, the dose can be less than about 100 mg/kg, less than about 90 mg/kg, less than about 80 mg/kg, less than about 70 mg/kg, less than about 60 mg/kg, less than about 50 mg/kg, less than about 40 mg/kg, less than about 30 mg/kg, less than about 25 mg/kg, less than about 20 mg/kg, less than about 10 mg/kg, less than about 7.5 mg/kg, less than about 6 mg/kg, less than about 5 mg/kg, less than about 4 mg/kg, less than about 3 mg/kg, less than about 2.5 mg/kg, less than about 1 mg/kg, less than about 0.5 mg/kg, less than about 0.1 mg/kg, less than about 0.05 mg/kg, or less than about 0.005 mg/kg of body weight. In some embodiments, the actual unit dose is 0.05 mg/kg of body weight, 0.07 mg/kg of body weight, 0.1 mg/kg of body weight, 0.3 mg/kg of body weight, 1.0 mg/kg of body weight, 3.0 mg/kg of body weight, 5.0 mg/kg of body weight, 10.0 mg/kg of body weight, or 25.0 mg/kg of body weight. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, or from about 20 mg to about 300 mg, or from about 200 mg to about 2000 mg. In some embodiments, the actual unit dose is about 0.1 mg. In some embodiments, the actual unit dose is about 0.5 mg. In some embodiments, the actual unit dose is about 1 mg. In some embodiments, the actual unit dose is about 1.5 mg. In some embodiments, the actual unit dose is about 2 mg. In some embodiments, the actual unit dose is about 2.5 mg. In some embodiments, the actual unit dose is about 3 mg. In some embodiments, the actual unit dose is about 3.5 mg. In some embodiments, the actual unit dose is about 4 mg. In some embodiments, the actual unit dose is about 4.5 mg. In some embodiments, the actual unit dose is about 5 mg. In some embodiments the actual unit dose is about 10 mg. In some embodiments, the actual unit dose is about 25 mg. In some embodiments, the actual unit dose is about 250 mg or less. In some embodiments, the actual unit dose is about 100 mg or less. In some embodiments, the actual unit dose is about 70 mg or less.

In some embodiments, the compound dose can be from about 0.1 mg to about 100 mg, from about 0.1 mg to about 50 mg, from about 0.1 mg to about 20 mg, from about 0.1 mg to about 10 mg, from about 0.5 mg to about 100 mg, from about 0.5 mg to about 50 mg, from about 0.5 mg to about 20 mg, from about 0.5 mg to about 10 mg, from about 1 mg to about 100 mg, from about 1 mg to about 50 mg, from about 1 mg to about 20 mg, from about 1 mg to about 10 mg, from about 2.5 mg to about 50 mg, from about 2.5 mg to about 20 mg, from about 2.5 mg to about 10 mg, or from about 2.5 mg to about 5 mg. In some embodiments, the compound dose is from about 5 mg to about 300 mg, from about 5 mg to about 200 mg, from about 7.5 mg to about 200 mg, from about 10 mg to about 100 mg, from about 15 mg to about 100 mg, from about 20 mg to about 100 mg, from about 30 mg to about 100 mg, from about 40 mg to about 100 mg, from about 10 mg to about 80 mg, from about 15 mg to about 80 mg, from about 20 mg to about 80 mg, from about 30 mg to about 80 mg, from about 40 mg to about 80 mg, from about 10 mg to about 60 mg, from about 15 mg to about 60 mg, from about 20 mg to about 60 mg, from about 30 mg to about 60 mg, or from about 40 mg to about 60 mg. In some embodiments, the compound administered is from about 20 mg to about 60 mg, from about 27 mg to about 60 mg, from about 20 mg to about 45 mg, or from about 27 mg to about 45 mg.

In some embodiments, the compound administered is from about 5 mg to about 7.5 mg, from about 5 mg to about 9 mg, from about 5 mg to about 10 mg, from about 5 mg to about 12 mg, from about 5 mg to about 14 mg, from about 5 mg to about 15 mg, from about 5 mg to about 16 mg, from about 5 mg to about 18 mg, from about 5 mg to about 20 mg, from about 5 mg to about 22 mg, from about 5 mg to about 24 mg, from about 5 mg to about 26 mg, from about 5 mg to about 28 mg, from about 5 mg to about 30 mg, from about 5 mg to about 32 mg, from about 5 mg to about 34 mg, from about 5 mg to about 36 mg, from about 5 mg to about 38 mg, from about 5 mg to about 40 mg, from about 5 mg to about 42 mg, from about 5 mg to about 44 mg, from about 5 mg to about 46 mg, from about 5 mg to about 48 mg, from about 5 mg to about 50 mg, from about 5 mg to about 52 mg, from about 5 mg to about 54 mg, from about 5 mg to about 56 mg, from about 5 mg to about 58 mg, from about 5 mg to about 60 mg, from about 10 mg to about 12 mg, from about 10 mg to about 14 mg, from about 10 mg to about 15 mg, from about 10 mg to about 16 mg, from about 10 mg to about 18 mg, from about 10 mg to about 20 mg, from about 10 mg to about 22 mg, from about 10 mg to about 24 mg, from about 10 mg to about 26 mg, from about 10 mg to about 28 mg, from about 10 mg to about 30 mg, from about 10 mg to about 32 mg, from about 10 mg to about 34 mg, from about 10 mg to about 36 mg, from about 10 mg to about 38 mg, from about 10 mg to about 40 mg, from about 10 mg to about 42 mg, from about 10 mg to about 44 mg, from about 10 mg to about 46 mg, from about 10 mg to about 48 mg, from about 10 mg to about 50 mg, from about 10 mg to about 52 mg, from about 10 mg to about 54 mg, from about 10 mg to about 56 mg, from about 10 mg to about 58 mg, from about 10 mg to about 60 mg, from about 15 mg to about 16 mg, from about 15 mg to about 18 mg, from about 15 mg to about 20 mg, from about 15 mg to about 22 mg, from about 15 mg to about 24 mg, from about 15 mg to about 26 mg, from about 15 mg to about 28 mg, from about 15 mg to about 30 mg, from about 15 mg to about 32 mg, from about 15 mg to about 34 mg, from about 15 mg to about 36 mg, from about 15 mg to about 38 mg, from about 15 mg to about 40 mg, from about 15 mg to about 42 mg, from about 15 mg to about 44 mg, from about 15 mg to about 46 mg, from about 15 mg to about 48 mg, from about 15 mg to about 50 mg, from about 15 mg to about 52 mg, from about 15 mg to about 54 mg, from about 15 mg to about 56 mg, from about 15 mg to about 58 mg, from about 15 mg to about 60 mg, from about 20 mg to about 22 mg, from about 20 mg to about 24 mg, from about 20 mg to about 26 mg, from about 20 mg to about 28 mg, from about 20 mg to about 30 mg, from about 20 mg to about 32 mg, from about 20 mg to about 34 mg, from about 20 mg to about 36 mg, from about 20 mg to about 38 mg, from about 20 mg to about 40 mg, from about 20 mg to about 42 mg, from about 20 mg to about 44 mg, from about 20 mg to about 46 mg, from about 20 mg to about 48 mg, from about 20 mg to about 50 mg, from about 20 mg to about 52 mg, from about 20 mg to about 54 mg, from about 20 mg to about 56 mg, from about 20 mg to about 58 mg, from about 20 mg to about 60 mg, from about 25 mg to about 26 mg, from about 25 mg to about 28 mg, from about 25 mg to about 30 mg, from about 25 mg to about 32 mg, from about 25 mg to about 34 mg, from about 25 mg to about 36 mg, from about 25 mg to about 38 mg, from about 25 mg to about 40 mg, from about 25 mg to about 42 mg, from about 25 mg to about 44 mg, from about 25 mg to about 46 mg, from about 25 mg to about 48 mg, from about 25 mg to about 50 mg, from about 25 mg to about 52 mg, from about 25 mg to about 54 mg, from about 25 mg to about 56 mg, from about 25 mg to about 58 mg, from about 25 mg to about 60 mg, from about 30 mg to about 32 mg, from about 30 mg to about 34 mg, from about 30 mg to about 36 mg, from about 30 mg to about 38 mg, from about 30 mg to about 40 mg, from about 30 mg to about 42 mg, from about 30 mg to about 44 mg, from about 30 mg to about 46 mg, from about 30 mg to about 48 mg, from about 30 mg to about 50 mg, from about 30 mg to about 52 mg, from about 30 mg to about 54 mg, from about 30 mg to about 56 mg, from about 30 mg to about 58 mg, from about 30 mg to about 60 mg, from about 40 mg to about 42 mg, from about 40 mg to about 44 mg, from about 40 mg to about 46 mg, from about 40 mg to about 48 mg, from about 40 mg to about 50 mg, from about 40 mg to about 52 mg, from about 40 mg to about 54 mg, from about 40 mg to about 56 mg, from about 40 mg to about 58 mg, from about 40 mg to about 60 mg, from about 45 mg to about 48 mg, from about 45 mg to about 50 mg, from about 45 mg to about 52 mg, from about 45 mg to about 54 mg, from about 45 mg to about 56 mg, from about 45 mg to about 58 mg, from about 45 mg to about 60 mg, from about 50 mg to about 52 mg, from about 50 mg to about 54 mg, from about 50 mg to about 56 mg, from about 50 mg to about 58 mg, or from about 50 mg to about 60 mg.

In some embodiments, the compound dose is greater than about 5 mg, greater than about 10 mg, greater than about 12.5 mg, greater than about 13.5 mg, greater than about 15 mg, greater than about 17.5 mg, greater than about 20 mg, greater than about 22.5 mg, greater than about 25 mg, greater than about 27 mg, greater than about 30 mg, greater than about 40 mg, greater than about 50 mg, greater than about 60 mg, greater than about 70 mg, greater than about 80 mg, greater than about 90 mg, greater than about 100 mg, greater than about 125 mg, greater than about 150 mg, or greater than about 200 mg. In some embodiments, the compound dose is less than about 5 mg, less than about 10 mg, less than about 12.5 mg, less than about 13.5 mg, less than about 15 mg, less than about 17.5 mg, less than about 20 mg, less than about 22.5 mg, less than about 25 mg, less than about 27 mg, less than about 30 mg, less than about 40 mg, less than about 50 mg, less than about 60 mg, less than about 70 mg, less than about 80 mg, less than about 90 mg, less than about 100 mg, less than about 125 mg, less than about 150 mg, or less than about 200 mg. In some embodiments, the compound dose is about 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, about 200 mg, about 225 mg, about 250 mg, about 275 mg, or about 300 mg.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in liver disease, a decrease/reduction in the severity of liver disease (such as, for example, a reduction or inhibition of development or liver disease), a decrease/reduction in symptoms and liver disease-related effects, delaying the onset of symptoms and liver disease-related effects, reducing the severity of symptoms of liver disease-related effects, reducing the severity of an acute episode, reducing the number of symptoms and liver disease-related effects, reducing the latency of symptoms and liver disease-related effects, an amelioration of symptoms and liver disease-related effects, reducing secondary symptoms, reducing secondary infections, preventing relapse to liver disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of liver disease development/progression (such as, for example, a complete or partial avoidance/inhibition or a delay), and an increased survival time of the affected host animal, following administration of a therapeutic protocol. Treatment of liver disease encompasses the treatment of subjects already diagnosed as having any form of liver disease at any clinical stage or manifestation, the delay of the onset or evolution or aggravation or deterioration of the symptoms or signs of liver disease, and/or preventing and/or reducing the severity of liver disease.

The present disclosure also provides methods of identifying a subject having an increased risk for developing liver disease. In some embodiments, the method comprises determining or having determined in a biological sample obtained from the subject the presence or absence of a GPAM predicted loss-of-function variant nucleic acid molecule (such as a genomic nucleic acid molecule, mRNA molecule, and/or cDNA molecule) encoding a human GPAM polypeptide. When the subject lacks a GPAM predicted loss-of-function variant nucleic acid molecule (i.e., the subject is genotypically categorized as a GPAM reference), then the subject has an increased risk for developing liver disease. When the subject has a GPAM predicted loss-of-function variant nucleic acid molecule (i.e., the subject is heterozygous or homozygous for a GPAM predicted loss-of-function variant), then the subject has a decreased risk for developing liver disease.

Having a single copy of a GPAM predicted loss-of-function variant nucleic acid molecule is more protective of a subject from developing liver disease than having no copies of a GPAM predicted loss-of-function variant nucleic acid molecule. Without intending to be limited to any particular theory or mechanism of action, it is believed that a single copy of a GPAM predicted loss-of-function variant nucleic acid molecule (i.e., heterozygous for a GPAM predicted loss-of-function variant) is protective of a subject from developing liver disease, and it is also believed that having two copies of a GPAM predicted loss-of-function variant nucleic acid molecule (i.e., homozygous for a GPAM predicted loss-of-function variant) may be more protective of a subject from developing liver disease, relative to a subject with a single copy. Thus, in some embodiments, a single copy of a GPAM predicted loss-of-function variant nucleic acid molecule may not be completely protective, but instead, may be partially or incompletely protective of a subject from developing liver disease. While not desiring to be bound by any particular theory, there may be additional factors or molecules involved in the development of liver disease that are still present in a subject having a single copy of a GPAM predicted loss-of-function variant nucleic acid molecule, thus resulting in less than complete protection from the development of liver disease.

Determining whether a subject has a GPAM predicted loss-of-function variant nucleic acid molecule in a biological sample from a subject and/or determining whether a subject has a GPAM predicted loss-of-function variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of identifying a subject having an increased risk of developing a liver disease wherein the methods comprise determining or having determined the subject's aggregate burden of having one or more GPAM predicted loss-of-function variant genomic nucleic acid molecules, mRNA molecules, or cDNA molecules described herein, and/or one or more GPAM predicted loss-of-function variant polypeptides described herein. The greater the aggregate burden the subject has, the lower the risk for developing a liver disease. The lower the aggregate burden the subject has, the greater the risk for developing a liver disease.

In some embodiments, the methods can further comprise determining the subject's aggregate burden of having a predicted loss-of-function variant GPAM genomic nucleic acid molecule, mRNA molecule, or cDNA molecule produced from an mRNA molecule, and/or a predicted loss-of-function variant GPAM polypeptide associated with a decreased risk of liver disease. The aggregate burden is the sum of all variants in the GPAM gene, which can be carried out in an association analysis with liver disease. In some embodiments, the subject is homozygous for one or more predicted loss-of-function variant GPAM nucleic acid molecules associated with a decreased risk of developing liver disease. In some embodiments, the subject is heterozygous for one or more predicted loss-of-function variant GPAM nucleic acid molecules associated with a decreased risk of developing liver disease. The result of the association analysis suggests that loss-of-function and missense variants of GPAM are associated with decreased risk of liver disease. In some embodiments, when a subject is identified as having an increased risk of developing a liver disease based on their aggregate burden, the subject is further treated with a therapeutic agent that treats or inhibits liver diseases and/or a GPAM inhibitor, as described herein.

In some embodiments, the subject's aggregate burden of having any one or more of GPAM predicted loss-of-function variant nucleic acid molecules represents a weighted sum of a plurality of any of the predicted loss-of-function variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, or at least about 1,000 of genetic variants associated with a liver disease. In some embodiments, when the subject has an aggregate burden above a desired threshold score, the subject has a lower or decreased risk of developing a liver disease. In some embodiments, when the subject has an aggregate burden below a desired threshold score, the subject has a greater or increased risk of developing a liver disease.

In some embodiments, the aggregate burden may be divided into quintiles, e.g., top quintile, intermediate quintile, and bottom quintile, wherein the top quintile of aggregate burden corresponds to the lowest risk group and the bottom quintile of aggregate burden corresponds to the highest risk group. In some embodiments, a subject having a greater aggregate burden comprise the highest weighted aggregate burdens, including, but not limited to the top 10%, top 20%, top 30%, top 40%, or top 50% of aggregate burdens from a subject population. In some embodiments, the genetic variants comprise the genetic variants having association with a liver disease in the top 10%, top 20%, top 30%, top 40%, or top 50% of p-value range for the association. In some embodiments, each of the identified genetic variants comprise the genetic variants having association with a liver disease with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with a liver disease with p-value of less than 5×10⁻⁸. In some embodiments, the identified genetic variants comprise genetic variants having association with a liver disease in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, or from about 6.5 to about 7.0. In some embodiments, high-risk subjects comprise subjects having aggregate burdens in the bottom decile, quintile, or tertile in a reference population.

In some embodiments, when a subject is identified as having an increased risk of developing liver disease, the subject is further treated with a therapeutic agent that treats or inhibits liver disease and/or a GPAM inhibitor, as described herein. For example, when the subject is GPAM reference, and therefore has an increased risk for developing liver disease, the subject is administered a GPAM inhibitor. In some embodiments, such a subject is also administered a therapeutic agent that treats or inhibits liver disease. In some embodiments, when the subject is heterozygous for a GPAM predicted loss-of-function variant, the subject is administered the therapeutic agent that treats or inhibits liver disease in a dosage amount that is the same as or lower than a standard dosage amount, and is also administered a GPAM inhibitor. In some embodiments, the subject is GPAM reference. In some embodiments, the subject is heterozygous for a GPAM predicted loss-of-function variant. Furthermore, when the subject has a lower aggregate burden for having a GPAM predicted loss-of-function variant nucleic acid molecule, and therefore has an increased risk for liver disease, the subject is administered a therapeutic agent that treats or inhibits liver disease. In some embodiments, when the subject has a lower aggregate burden for having a GPAM predicted loss-of-function variant nucleic acid molecule, the subject is administered the therapeutic agent that treats or inhibits liver disease in a dosage amount that is the same as or greater than the standard dosage amount administered to a subject who has a greater aggregate burden for having a GPAM predicted loss-of-function variant nucleic acid molecule.

The present disclosure also provides methods of detecting the presence or absence of a GPAM predicted loss-of-function variant genomic nucleic acid molecule in a biological sample from a subject, and/or a GPAM predicted loss-of-function variant mRNA molecule in a biological sample from a subject, and/or a GPAM predicted loss-of-function variant cDNA molecule produced from an mRNA molecule in a biological sample from a subject. It is understood that gene sequences within a population and mRNA molecules encoded by such genes can vary due to polymorphisms such as single-nucleotide polymorphisms. The sequences provided herein for the GPAM variant genomic nucleic acid molecule, GPAM variant mRNA molecule, and GPAM variant cDNA molecule are only exemplary sequences. Other sequences for the GPAM variant genomic nucleic acid molecule, variant mRNA molecule, and variant cDNA molecule are also possible.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting any GPAM variant nucleic acid molecule, preliminary processing designed to isolate or enrich the sample for the genomic DNA can be employed. A variety of techniques may be used for this purpose. When detecting the level of any GPAM variant mRNA, different techniques can be used enrich the biological sample with mRNA. Various methods to detect the presence or level of an mRNA or the presence of a particular variant genomic DNA locus can be used.

In some embodiments, detecting a human GPAM predicted loss-of-function variant nucleic acid molecule in a subject comprises assaying or analyzing a biological sample obtained from the subject to determine whether a GPAM genomic nucleic acid molecule in the biological sample, and/or a GPAM mRNA molecule in the biological sample, and/or a GPAM cDNA molecule produced from an mRNA 36 molecule in the biological sample, comprises one or more variations that cause a loss-of-function (partial or complete) or are predicted to cause a loss-of-function (partial or complete).

In some embodiments, the methods of detecting the presence or absence of a GPAM predicted loss-of-function variant nucleic acid molecule (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the nucleotide sequence comprises a guanine at a position corresponding to: position 3,195 according to SEQ ID NO:2 (for genomic nucleic acid molecules), position 327 according to SEQ ID NO:9 (for mRNA molecules), or position 327 according to SEQ ID NO:21 (for cDNA molecules obtained from mRNA molecules).

In some embodiments, the nucleotide sequence comprises a guanine at a position corresponding to: position 291 according to SEQ ID NO:10 (for mRNA molecules), or position 291 according to SEQ ID NO:22 (for cDNA molecules obtained from mRNA molecules).

In some embodiments, the nucleotide sequence comprises a guanine at a position corresponding to: position 323 according to SEQ ID NO:11 (for mRNA molecules), or position 323 according to SEQ ID NO:23 (for cDNA molecules obtained from mRNA molecules).

In some embodiments, the nucleotide sequence comprises a guanine at a position corresponding to: position 326 according to SEQ ID NO:12 (for mRNA molecules), or position 326 according to SEQ ID NO:24 (for cDNA molecules obtained from mRNA molecules).

In some embodiments, the nucleotide sequence comprises a guanine at a position corresponding to: position 305 according to SEQ ID NO:13 (for mRNA molecules), or position 305 according to SEQ ID NO:25 (for cDNA molecules obtained from mRNA molecules).

In some embodiments, the nucleotide sequence comprises a guanine at a position corresponding to: position 170 according to SEQ ID NO:14 (for mRNA molecules), or position 170 according to SEQ ID NO:26 (for cDNA molecules obtained from mRNA molecules).

In some embodiments, the nucleotide sequence comprises a guanine at a position corresponding to position 3,195 according to SEQ ID NO:2, or the complement thereof.

In some embodiments, the nucleotide sequence comprises a guanine at a position corresponding to: position 327 according to SEQ ID NO:9, or the complement thereof; position 291 according to SEQ ID NO:10, or the complement thereof; position 323 according to SEQ ID NO:11, or the complement thereof; position 326 according to SEQ ID NO:12, or the complement thereof; position 305 according to SEQ ID NO:13, or the complement thereof; or position 170 according to SEQ ID NO:14, or the complement thereof.

In some embodiments, the nucleotide sequence comprises a guanine at a position corresponding to: position 327 according to SEQ ID NO:21, or the complement thereof; position 291 according to SEQ ID NO:22, or the complement thereof; position 323 according to SEQ ID NO:23, or the complement thereof; position 326 according to SEQ ID NO:24, or the complement thereof; position 305 according to SEQ ID NO:25, or the complement thereof; or position 170 according to SEQ ID NO:26, or the complement thereof.

In some embodiments, the biological sample comprises a cell or cell lysate. Such methods can further comprise, for example, obtaining a biological sample from the subject comprising a GPAM genomic nucleic acid molecule or mRNA molecule, and if mRNA, optionally reverse transcribing the mRNA into cDNA. Such assays can comprise, for example determining the identity of these positions of the particular GPAM nucleic acid molecule. In some embodiments, the method is an in vitro method.

In some embodiments, the determining step, detecting step, or sequence analysis comprises sequencing at least a portion of the nucleotide sequence of the GPAM genomic nucleic acid molecule, the GPAM mRNA molecule, or the GPAM cDNA molecule in the biological sample, wherein the sequenced portion comprises one or more variations that cause a loss-of-function (partial or complete) or are predicted to cause a loss-of-function (partial or complete).

In some embodiments, the determining step, detecting step, or sequence analysis comprises sequencing at least a portion of: the nucleotide sequence of the GPAM genomic nucleic acid molecule in the biological sample, wherein the sequenced portion comprises a position corresponding to position 3,195 according to SEQ ID NO:2, or the complement thereof; the nucleotide sequence of the GPAM mRNA molecule in the biological sample, wherein the sequenced portion comprises a position corresponding to position 327 according to SEQ ID NO:9, or the complement thereof; and/or the nucleotide sequence of the GPAM cDNA molecule produced from the mRNA in the biological sample, wherein the sequenced portion comprises a position corresponding to position 327 according to SEQ ID NO:21, or the complement thereof. When the sequenced portion of the GPAM nucleic acid molecule in the biological sample comprises a guanine at a position corresponding to: position 3,195 according to SEQ ID NO:2, position 327 according to SEQ ID NO:9, or position 327 according to SEQ ID NO:21, then the GPAM nucleic acid molecule in the biological sample is a GPAM predicted loss-of-function variant nucleic acid molecule.

In some embodiments, the determining step, detecting step, or sequence analysis comprises sequencing at least a portion of: the nucleotide sequence of the GPAM mRNA molecule in the biological sample, wherein the sequenced portion comprises a position corresponding to position 291 according to SEQ ID NO:10, or the complement thereof; and/or the nucleotide sequence of the GPAM cDNA molecule produced from the mRNA in the biological sample, wherein the sequenced portion comprises a position corresponding to position 291 according to SEQ ID NO:22, or the complement thereof. When the sequenced portion of the GPAM nucleic acid molecule in the biological sample comprises a guanine at a position corresponding to: position 291 according to SEQ ID NO:10, or position 291 according to SEQ ID NO:22, then the GPAM nucleic acid molecule in the biological sample is a GPAM predicted loss-of-function variant nucleic acid molecule.

In some embodiments, the determining step, detecting step, or sequence analysis comprises sequencing at least a portion of: the nucleotide sequence of the GPAM mRNA molecule in the biological sample, wherein the sequenced portion comprises a position corresponding to position 323 according to SEQ ID NO:11, or the complement thereof; and/or the nucleotide sequence of the GPAM cDNA molecule produced from the mRNA in the biological sample, wherein the sequenced portion comprises a position corresponding to position 323 according to SEQ ID NO:23, or the complement thereof. When the sequenced portion of the GPAM nucleic acid molecule in the biological sample comprises a guanine at a position corresponding to: position 323 according to SEQ ID NO:11, or position 323 according to SEQ ID NO:23, then the GPAM nucleic acid molecule in the biological sample is a GPAM predicted loss-of-function variant nucleic acid molecule.

In some embodiments, the determining step, detecting step, or sequence analysis comprises sequencing at least a portion of: the nucleotide sequence of the GPAM mRNA molecule in the biological sample, wherein the sequenced portion comprises a position corresponding to position 326 according to SEQ ID NO:12, or the complement thereof; and/or the nucleotide sequence of the GPAM cDNA molecule produced from the mRNA in the biological sample, wherein the sequenced portion comprises a position corresponding to position 326 according to SEQ ID NO:24, or the complement thereof. When the sequenced portion of the GPAM nucleic acid molecule in the biological sample comprises a guanine at a position corresponding to: position 326 according to SEQ ID NO:12, or position 326 according to SEQ ID NO:24, then the GPAM nucleic acid molecule in the biological sample is a GPAM predicted loss-of-function variant nucleic acid molecule.

In some embodiments, the determining step, detecting step, or sequence analysis comprises sequencing at least a portion of: the nucleotide sequence of the GPAM mRNA molecule in the biological sample, wherein the sequenced portion comprises a position corresponding to position 305 according to SEQ ID NO:13, or the complement thereof; and/or the nucleotide sequence of the GPAM cDNA molecule produced from the mRNA in the biological sample, wherein the sequenced portion comprises a position corresponding to position 305 according to SEQ ID NO:25, or the complement thereof. When the sequenced portion of the GPAM nucleic acid molecule in the biological sample comprises a guanine at a position corresponding to: position 305 according to SEQ ID NO:13, or position 305 according to SEQ ID NO:25, then the GPAM nucleic acid molecule in the biological sample is a GPAM predicted loss-of-function variant nucleic acid molecule.

In some embodiments, the determining step, detecting step, or sequence analysis comprises sequencing at least a portion of: the nucleotide sequence of the GPAM mRNA molecule in the biological sample, wherein the sequenced portion comprises a position corresponding to position 170 according to SEQ ID NO:14, or the complement thereof; and/or the nucleotide sequence of the GPAM cDNA molecule produced from the mRNA in the biological sample, wherein the sequenced portion comprises a position corresponding to position 170 according to SEQ ID NO:26, or the complement thereof. When the sequenced portion of the GPAM nucleic acid molecule in the biological sample comprises a guanine at a position corresponding to: position 170 according to SEQ ID NO:14, or position 170 according to SEQ ID NO:26, then the GPAM nucleic acid molecule in the biological sample is a GPAM predicted loss-of-function variant nucleic acid molecule.

In some embodiments, the determining step, detecting step, or sequence analysis comprises sequencing at least a portion of the nucleotide sequence of the GPAM genomic nucleic acid molecule in the biological sample, wherein the sequenced portion comprises a position corresponding to: position 3,195 according to SEQ ID NO:2, or the complement thereof, then the GPAM nucleic acid molecule in the biological sample is a GPAM predicted loss-of-function variant nucleic acid molecule.

In some embodiments, the determining step, detecting step, or sequence analysis comprises sequencing at least a portion of the nucleotide sequence of the GPAM mRNA molecule in the biological sample, wherein the sequenced portion comprises a position corresponding to: position 327 according to SEQ ID NO:9, or the complement thereof; position 291 according to SEQ ID NO:10, or the complement thereof; position 323 according to SEQ ID NO:11, or the complement thereof; position 326 according to SEQ ID NO:12, or the complement thereof; position 305 according to SEQ ID NO:13, or the complement thereof; or position 170 according to SEQ ID NO:14, or the complement thereof. When the sequenced portion of the GPAM nucleic acid molecule in the biological sample comprises a guanine at a position corresponding to: position 327 according to SEQ ID NO:9, position 291 according to SEQ ID NO:10, position 323 according to SEQ ID NO:11, position 326 according to SEQ ID NO:12, position 305 according to SEQ ID NO:13, or position 170 according to SEQ ID NO:14, then the GPAM nucleic acid molecule in the biological sample is a GPAM predicted loss-of-function variant nucleic acid molecule.

In some embodiments, the determining step, detecting step, or sequence analysis comprises sequencing at least a portion of the nucleotide sequence of the GPAM cDNA molecule in the biological sample, wherein the sequenced portion comprises a position corresponding to: position 327 according to SEQ ID NO:21, or the complement thereof; position 291 according to SEQ ID NO:22, or the complement thereof; position 323 according to SEQ ID NO:23, or the complement thereof; position 326 according to SEQ ID NO:24, or the complement thereof; position 305 according to SEQ ID NO:25, or the complement thereof; or position 170 according to SEQ ID NO:26, or the complement thereof. When the sequenced portion of the GPAM nucleic acid molecule in the biological sample comprises a guanine at a position corresponding to: position 327 according to SEQ ID NO:21, position 291 according to SEQ ID NO:22, position 323 according to SEQ ID NO:23, position 326 according to SEQ ID NO:24, position 305 according to SEQ ID NO:25, or position 170 according to SEQ ID NO:26; then the GPAM nucleic acid molecule in the biological sample is a GPAM predicted loss-of-function variant nucleic acid molecule.

In some embodiments, the determining step, detecting step, or sequence analysis comprises: a) contacting the biological sample with a primer hybridizing to a portion of the nucleotide sequence of the GPAM: genomic nucleic acid molecule that is proximate to a position corresponding to position 3,195 according to SEQ ID NO:2; mRNA molecule that is proximate to a position corresponding to position 327 according to SEQ ID NO:9; and/or cDNA molecule that is proximate to a position corresponding to position 327 according to SEQ ID NO:21; b) extending the primer at least through the position of the nucleotide sequence of the GPAM: genomic nucleic acid molecule corresponding to position 3,195 according to SEQ ID NO:2; mRNA molecule corresponding to position 327 according to SEQ ID NO:9; and/or cDNA molecule corresponding to position 327 according to SEQ ID NO:21; and c) determining whether the extension product of the primer comprises a guanine at a position corresponding to; position 3,195 according to SEQ ID NO:2, position 327 according to SEQ ID NO:9, and/or position 327 according to SEQ ID NO:21.

In some embodiments, the determining step, detecting step, or sequence analysis comprises: a) contacting the biological sample with a primer hybridizing to a portion of the nucleotide sequence of the GPAM: mRNA molecule that is proximate to a position corresponding to position 291 according to SEQ ID NO:10; and/or cDNA molecule that is proximate to a position corresponding to position 291 according to SEQ ID NO:22; b) extending the primer at least through the position of the nucleotide sequence of the GPAM: mRNA molecule corresponding to position 291 according to SEQ ID NO:10; and/or cDNA molecule corresponding to position 291 according to SEQ ID NO:22; and c) determining whether the extension product of the primer comprises a guanine at a position corresponding to: position 291 according to SEQ ID NO:10, and/or position 291 according to SEQ ID NO:22.

In some embodiments, the determining step, detecting step, or sequence analysis comprises: a) contacting the biological sample with a primer hybridizing to a portion of the nucleotide sequence of the GPAM: mRNA molecule that is proximate to a position corresponding to position 323 according to SEQ ID NO:11; and/or cDNA molecule that is proximate to a position corresponding to position 323 according to SEQ ID NO:23; b) extending the primer at least through the position of the nucleotide sequence of the GPAM: mRNA molecule corresponding to position 323 according to SEQ ID NO:11; and/or cDNA molecule corresponding to position 323 according to SEQ ID NO:23; and c) determining whether the extension product of the primer comprises a guanine at a position corresponding to: position 323 according to SEQ ID NO:11, and/or position 323 according to SEQ ID NO:23.

In some embodiments, the determining step, detecting step, or sequence analysis comprises: a) contacting the biological sample with a primer hybridizing to a portion of the nucleotide sequence of the GPAM: mRNA molecule that is proximate to a position corresponding to position 326 according to SEQ ID NO:12; and/or cDNA molecule that is proximate to a position corresponding to position 326 according to SEQ ID NO:24; b) extending the primer at least through the position of the nucleotide sequence of the GPAM: mRNA molecule corresponding to position 326 according to SEQ ID NO:12; and/or cDNA molecule corresponding to position 326 according to SEQ ID NO:24; and c) determining whether the extension product of the primer comprises a guanine at a position corresponding to: position 326 according to SEQ ID NO:12, and/or position 326 according to SEQ ID NO:24.

In some embodiments, the determining step, detecting step, or sequence analysis comprises: a) contacting the biological sample with a primer hybridizing to a portion of the nucleotide sequence of the GPAM: mRNA molecule that is proximate to a position corresponding to position 305 according to SEQ ID NO:13; and/or cDNA molecule that is proximate to a position corresponding to position 305 according to SEQ ID NO:25; b) extending the primer at least through the position of the nucleotide sequence of the GPAM: mRNA molecule corresponding to position 305 according to SEQ ID NO:13; and/or cDNA molecule corresponding to position 305 according to SEQ ID NO:25; and c) determining whether the extension product of the primer comprises a guanine at a position corresponding to: position 305 according to SEQ ID NO:13, and/or position 305 according to SEQ ID NO:25.

In some embodiments, the determining step, detecting step, or sequence analysis comprises: a) contacting the biological sample with a primer hybridizing to a portion of the nucleotide sequence of the GPAM: mRNA molecule that is proximate to a position corresponding to position 170 according to SEQ ID NO:14; and/or cDNA molecule that is proximate to a position corresponding to position 170 according to SEQ ID NO:26; b) extending the primer at least through the position of the nucleotide sequence of the GPAM: mRNA molecule corresponding to position 170 according to SEQ ID NO:14; and/or cDNA molecule corresponding to position 170 according to SEQ ID NO:26; and c) determining whether the extension product of the primer comprises a guanine at a position corresponding to: position 170 according to SEQ ID NO:14, and/or position 170 according to SEQ ID NO:26.

In some embodiments, the determining step, detecting step, or sequence analysis comprises: a) contacting the biological sample with a primer hybridizing to a portion of the nucleotide sequence of the GPAM genomic nucleic acid molecule that is proximate to a position corresponding to position 3,195 according to SEQ ID NO:2; b) extending the primer at least through the position of the nucleotide sequence of the GPAM genomic nucleic acid molecule corresponding to position 3,195 according to SEQ ID NO:2; and c) determining whether the extension product of the primer comprises a guanine at a position corresponding to position 3,195 according to SEQ ID NO:2.

In some embodiments, the determining step, detecting step, or sequence analysis comprises: a) contacting the biological sample with a primer hybridizing to a portion of the nucleotide sequence of the GPAM mRNA molecule that is proximate to a position corresponding to: position 327 according to SEQ ID NO:9, position 291 according to SEQ ID NO:10, position 323 according to SEQ ID NO:11, position 326 according to SEQ ID NO:12, position 305 according to SEQ ID NO:13, or position 170 according to SEQ ID NO:14; b) extending the primer at least through the position of the nucleotide sequence of the GPAM mRNA molecule corresponding to: position 327 according to SEQ ID NO:9, position 291 according to SEQ ID NO:10, position 323 according to SEQ ID NO:11, position 326 according to SEQ ID NO:12, position 305 according to SEQ ID NO:13, or position 170 according to SEQ ID NO:14; and c) determining whether the extension product of the primer comprises a guanine at a position corresponding to: position 327 according to SEQ ID NO:9, position 291 according to SEQ ID NO:10, position 323 according to SEQ ID NO:11, position 326 according to SEQ ID NO:12, position 305 according to SEQ ID NO:13, or position 170 according to SEQ ID NO:14.

In some embodiments, the determining step, detecting step, or sequence analysis comprises: a) contacting the biological sample with a primer hybridizing to a portion of the nucleotide sequence of the GPAM cDNA molecule that is proximate to a position corresponding to: position 327 according to SEQ ID NO:21, position 291 according to SEQ ID NO:22, position 323 according to SEQ ID NO:23, position 326 according to SEQ ID NO:24, position 305 according to SEQ ID NO:25, or position 170 according to SEQ ID NO:26; b) extending the primer at least through the position of the nucleotide sequence of the GPAM cDNA molecule corresponding to: position 327 according to SEQ ID NO:21, position 291 according to SEQ ID NO:22, position 323 according to SEQ ID NO:23, position 326 according to SEQ ID NO:24, position 305 according to SEQ ID NO:25, or position 170 according to SEQ ID NO:26; and c) determining whether the extension product of the primer comprises a guanine at a position corresponding to: position 327 according to SEQ ID NO:21, position 291 according to SEQ ID NO:22, position 323 according to SEQ ID NO:23, position 326 according to SEQ ID NO:24, position 305 according to SEQ ID NO:25, or position 170 according to SEQ ID NO:26.

In some embodiments, the assay comprises sequencing the entire nucleic acid molecule. In some embodiments, only a GPAM genomic nucleic acid molecule is analyzed. In some embodiments, only a GPAM mRNA is analyzed. In some embodiments, only a GPAM cDNA obtained from GPAM mRNA is analyzed.

In some embodiments, the determining step, detecting step, or sequence analysis comprises: a) amplifying at least a portion of the nucleic acid molecule that encodes the human GPAM polypeptide, wherein the amplified portion comprises a guanine at a position corresponding to: position 3,195 according to SEQ ID NO:2, or the complement thereof; position 327 according to SEQ ID NO:9, or the complement thereof; and/or position 327 according to SEQ ID NO:21, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to: position 3,195 according to SEQ ID NO:2, or the complement thereof; position 327 according to SEQ ID NO:9, or the complement thereof; and/or position 327 according to SEQ ID NO:21, or the complement thereof; and d) detecting the detectable label.

In some embodiments, the determining step, detecting step, or sequence analysis comprises: a) amplifying at least a portion of the nucleic acid molecule that encodes the human GPAM polypeptide, wherein the amplified portion comprises a guanine at a position corresponding to: position 291 according to SEQ ID NO:10, or the complement thereof; and/or position 291 according to SEQ ID NO:22, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to: position 291 according to SEQ ID NO:10, or the complement thereof; and/or position 291 according to SEQ ID NO:22, or the complement thereof; and d) detecting the detectable label.

In some embodiments, the determining step, detecting step, or sequence analysis comprises: a) amplifying at least a portion of the nucleic acid molecule that encodes the human GPAM polypeptide, wherein the amplified portion comprises a guanine at a position corresponding to: position 323 according to SEQ ID NO:11, or the complement thereof; and/or position 323 according to SEQ ID NO:23, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to: position 323 according to SEQ ID NO:11, or the complement thereof; and/or position 323 according to SEQ ID NO:23, or the complement thereof; and d) detecting the detectable label.

In some embodiments, the determining step, detecting step, or sequence analysis comprises: a) amplifying at least a portion of the nucleic acid molecule that encodes the human GPAM polypeptide, wherein the amplified portion comprises a guanine at a position corresponding to: position 326 according to SEQ ID NO:12, or the complement thereof; and/or position 326 according to SEQ ID NO:24, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to: position 326 according to SEQ ID NO:12, or the complement thereof; and/or position 326 according to SEQ ID NO:24, or the complement thereof; and d) detecting the detectable label.

In some embodiments, the determining step, detecting step, or sequence analysis comprises: a) amplifying at least a portion of the nucleic acid molecule that encodes the human GPAM polypeptide, wherein the amplified portion comprises a guanine at a position corresponding to: position 305 according to SEQ ID NO:13, or the complement thereof; and/or position 305 according to SEQ ID NO:25, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to: position 305 according to SEQ ID NO:13, or the complement thereof; and/or position 305 according to SEQ ID NO:25, or the complement thereof; and d) detecting the detectable label.

In some embodiments, the determining step, detecting step, or sequence analysis comprises: a) amplifying at least a portion of the nucleic acid molecule that encodes the human GPAM polypeptide, wherein the amplified portion comprises a guanine at a position corresponding to: position 170 according to SEQ ID NO:14, or the complement thereof; and/or position 170 according to SEQ ID NO:26, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to: position 170 according to SEQ ID NO:14, or the complement thereof; and/or position 170 according to SEQ ID NO:26, or the complement thereof; and d) detecting the detectable label.

In some embodiments, the determining step, detecting step, or sequence analysis comprises: a) amplifying at least a portion of the nucleic acid molecule that encodes the human GPAM polypeptide, wherein the amplified portion comprises a guanine at a position corresponding to position 3,195 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to position 3,195 according to SEQ ID NO:2, or the complement thereof; and d) detecting the detectable label.

In some embodiments, the determining step, detecting step, or sequence analysis comprises: a) amplifying at least a portion of the nucleic acid molecule that encodes the human GPAM polypeptide, wherein the amplified portion comprises a guanine at a position corresponding to: position 327 according to SEQ ID NO:9, or the complement thereof; position 291 according to SEQ ID NO:10, or the complement thereof; position 323 according to SEQ ID NO:11, or the complement thereof; position 326 according to SEQ ID NO:12, or the complement thereof; position 305 according to SEQ ID NO:13, or the complement thereof; or position 170 according to SEQ ID NO:14, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to: position 327 according to SEQ ID NO:9, or the complement thereof; position 291 according to SEQ ID NO:10, or the complement thereof; position 323 according to SEQ ID NO:11, or the complement thereof; position 326 according to SEQ ID NO:12, or the complement thereof; position 305 according to SEQ ID NO:13, or the complement thereof; or position 170 according to SEQ ID NO:14, or the complement thereof; and d) detecting the detectable label.

In some embodiments, the determining step, detecting step, or sequence analysis comprises: a) amplifying at least a portion of the nucleic acid molecule that encodes the human GPAM polypeptide, wherein the amplified portion comprises a guanine at a position corresponding to: position 327 according to SEQ ID NO:21, or the complement thereof; position 291 according to SEQ ID NO:22, or the complement thereof; position 323 according to SEQ ID NO:23, or the complement thereof; position 326 according to SEQ ID NO:24, or the complement thereof; position 305 according to SEQ ID NO:25, or the complement thereof; or position 170 according to SEQ ID NO:26, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a guanine at a position corresponding to: position 327 according to SEQ ID NO:21, or the complement thereof; position 291 according to SEQ ID NO:22, or the complement thereof; position 323 according to SEQ ID NO:23, or the complement thereof; position 326 according to SEQ ID NO:24, or the complement thereof; position 305 according to SEQ ID NO:25, or the complement thereof; or position 170 according to SEQ ID NO:26, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a guanine at a position corresponding to: position 3,195 according to SEQ ID NO:2, or the complement thereof; position 327 according to SEQ ID NO:9, or the complement thereof; and/or position 327 according to SEQ ID NO:21, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a guanine at a position corresponding to: position 291 according to SEQ ID NO:10, or the complement thereof; and/or position 291 according to SEQ ID NO:22, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a guanine at a position corresponding to: position 323 according to SEQ ID NO:11, or the complement thereof; and/or position 323 according to SEQ ID NO:23, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a guanine at a position corresponding to: position 326 according to SEQ ID NO:12, or the complement thereof; and/or position 326 according to SEQ ID NO:24, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a guanine at a position corresponding to: position 305 according to SEQ ID NO:13, or the complement thereof; and/or position 305 according to SEQ ID NO:25, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a guanine at a position corresponding to: position 170 according to SEQ ID NO:14, or the complement thereof; and/or position 170 according to SEQ ID NO:26, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a guanine at a position corresponding to position 3,195 according to SEQ ID NO:2, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a guanine at a position corresponding to: position 327 according to SEQ ID NO:9, or the complement thereof; position 291 according to SEQ ID NO:10, or the complement thereof; position 323 according to SEQ ID NO:11, or the complement thereof; position 326 according to SEQ ID NO:12, or the complement thereof; position 305 according to SEQ ID NO:13, or the complement thereof; or position 170 according to SEQ ID NO:14, or the complement thereof; and detecting the detectable label.

In some embodiments, the determining step, detecting step, or sequence analysis comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a guanine at a position corresponding to: position 327 according to SEQ ID NO:21, or the complement thereof; position 291 according to SEQ ID NO:22, or the complement thereof; position 323 according to SEQ ID NO:23, or the complement thereof; position 326 according to SEQ ID NO:24, or the complement thereof; position 305 according to SEQ ID NO:25, or the complement thereof; or position 170 according to SEQ ID NO:26, or the complement thereof; and detecting the detectable label.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to a GPAM variant genomic sequence, variant mRNA sequence, or variant cDNA sequence and not the corresponding GPAM reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a GPAM variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether a GPAM nucleic acid molecule (genomic nucleic acid molecule, mRNA molecule, or cDNA molecule), or complement thereof, within a biological sample comprises a nucleotide sequence comprising a guanine at a position corresponding to position 3,195 according to SEQ ID NO:2 (genomic nucleic acid molecule), or a guanine at a position corresponding to position 327 according to SEQ ID NO:9 (mRNA molecule), or a guanine at a position corresponding to position 327 according to SEQ ID NO:21 (cDNA molecule), the biological sample can be subjected to an amplification method using a primer pair that includes a first primer derived from the 5′ flanking sequence adjacent to a guanine at a position corresponding to position 3,195 according to SEQ ID NO:2, or a guanine at a position corresponding to position 327 according to SEQ ID NO:9, or a guanine at a position corresponding to position 327 according to SEQ ID NO:21, and a second primer derived from the 3′ flanking sequence adjacent to a guanine at a position corresponding to position 3,195 according to SEQ ID NO:2, or a guanine at a position corresponding to position 327 according to SEQ ID NO:9, or a guanine at a position corresponding to position 327 according to SEQ ID NO:21 to produce an amplicon that is indicative of the presence of the SNP at positions encoding a guanine at a position corresponding to position 3,195 according to SEQ ID NO:2, or a guanine at a position corresponding to position 327 according to SEQ ID NO:9, or a guanine at a position corresponding to position 327 according to SEQ ID NO:21. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a guanine at a position corresponding to position 3,195 according to SEQ ID NO:2, or a guanine at a position corresponding to position 327 according to SEQ ID NO:9, or a guanine at a position corresponding to position 327 according to SEQ ID NO:21, and at least 1, 2, 3, 4, 5, 6, 7, 8, 9, 10, or more nucleotides on each side of positions comprising a guanine at a position corresponding to position 3,195 according to SEQ ID NO:2, or a guanine at a position corresponding to position 327 according to SEQ ID NO:9, or a guanine at a position corresponding to position 327 according to SEQ ID NO:21.

In some embodiments, to determine whether a GPAM nucleic acid molecule (mRNA molecule or cDNA molecule), or complement thereof, within a biological sample comprises a guanine at a position corresponding to position 291 according to SEQ ID NO:10 (mRNA molecule), or a guanine at a position corresponding to position 291 according to SEQ ID NO:22 (cDNA molecule), the biological sample can be subjected to an amplification method using a primer pair that includes a first primer derived from the 5′ flanking sequence adjacent to a guanine at a position corresponding to position 291 according to SEQ ID NO:10, or a guanine at a position corresponding to position 291 according to SEQ ID NO:22, and a second primer derived from the 3′ flanking sequence adjacent to a guanine at a position corresponding to position 291 according to SEQ ID NO:10, or a guanine at a position corresponding to position 291 according to SEQ ID NO:22 to produce an amplicon that is indicative of the presence of the SNP at positions encoding a guanine at a position corresponding to position 291 according to SEQ ID NO:10, or a guanine at a position corresponding to position 291 according to SEQ ID NO:22. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a guanine at a position corresponding to position 291 according to SEQ ID NO:10, or a guanine at a position corresponding to position 291 according to SEQ ID NO:22, and at least 1, 2, 3, 4, 5, 6, 7, 8, 9, 10, or more nucleotides on each side of positions comprising a guanine at a position corresponding to position 291 according to SEQ ID NO:10, or a guanine at a position corresponding to position 291 according to SEQ ID NO:22.

In some embodiments, to determine whether a GPAM nucleic acid molecule (mRNA molecule or cDNA molecule), or complement thereof, within a biological sample comprises a nucleotide sequence comprising a guanine at a position corresponding to position 323 according to SEQ ID NO:11 (mRNA molecule), or a guanine at a position corresponding to position 323 according to SEQ ID NO:23 (cDNA molecule), the biological sample can be subjected to an amplification method using a primer pair that includes a first primer derived from the 5′ flanking sequence adjacent to a guanine at a position corresponding to position 323 according to SEQ ID NO:11, or a guanine at a position corresponding to position 323 according to SEQ ID NO:23, and a second primer derived from the 3′ flanking sequence adjacent to a guanine at a position corresponding to position 323 according to SEQ ID NO:23 to produce an amplicon that is indicative of the presence of the SNP at positions encoding a guanine at a position corresponding to position 323 according to SEQ ID NO:11, or a guanine at a position corresponding to position 323 according to SEQ ID NO:23. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a guanine at a position corresponding to position 323 according to SEQ ID NO:11, or a guanine at a position corresponding to position 323 according to SEQ ID NO:23, and at least 1, 2, 3, 4, 5, 6, 7, 8, 9, 10, or more nucleotides on each side of positions comprising a guanine at a position corresponding to position 323 according to SEQ ID NO:11, or a guanine at a position corresponding to position 323 according to SEQ ID NO:23.

In some embodiments, to determine whether a GPAM nucleic acid molecule (mRNA molecule or cDNA molecule), or complement thereof, within a biological sample comprises a nucleotide sequence comprising a guanine at a position corresponding to position 326 according to SEQ ID NO:12 (mRNA molecule), or a guanine at a position corresponding to position 326 according to SEQ ID NO:24 (cDNA molecule), the biological sample can be subjected to an amplification method using a primer pair that includes a first primer derived from the 5′ flanking sequence adjacent to a guanine at a position corresponding to position 326 according to SEQ ID NO:12, or a guanine at a position corresponding to position 326 according to SEQ ID NO:24, and a second primer derived from the 3′ flanking sequence adjacent to a guanine at a position corresponding to position 326 according to SEQ ID NO:12, or a guanine at a position corresponding to position 326 according to SEQ ID NO:24 to produce an amplicon that is indicative of the presence of the SNP at positions encoding a guanine at a position corresponding to position 326 according to SEQ ID NO:12, or a guanine at a position corresponding to position 326 according to SEQ ID NO:24. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a guanine at a position corresponding to position 326 according to SEQ ID NO:12, or a guanine at a position corresponding to position 326 according to SEQ ID NO:24, and at least 1, 2, 3, 4, 5, 6, 7, 8, 9, 10, or more nucleotides on each side of positions comprising a guanine at a position corresponding to position 326 according to SEQ ID NO:12, or a guanine at a position corresponding to position 326 according to SEQ ID NO:24.

In some embodiments, to determine whether a GPAM nucleic acid molecule (mRNA molecule or cDNA molecule), or complement thereof, within a biological sample comprises a nucleotide sequence comprising a guanine at a position corresponding to position 305 according to SEQ ID NO:13 (mRNA molecule), or a guanine at a position corresponding to position 305 according to SEQ ID NO:25 (cDNA molecule), the biological sample can be subjected to an amplification method using a primer pair that includes a first primer derived from the 5′ flanking sequence adjacent to a guanine at a position corresponding to position 305 according to SEQ ID NO:13, or a guanine at a position corresponding to position 305 according to SEQ ID NO:25, and a second primer derived from the 3′ flanking sequence adjacent to a guanine at a position corresponding to position 305 according to SEQ ID NO:13, or a guanine at a position corresponding to position 305 according to SEQ ID NO:25 to produce an amplicon that is indicative of the presence of the SNP at positions encoding a guanine at a position corresponding to position 305 according to SEQ ID NO:13, or a guanine at a position corresponding to position 305 according to SEQ ID NO:25. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a guanine at a position corresponding to position 305 according to SEQ ID NO:13, or a guanine at a position corresponding to position 305 according to SEQ ID NO:25, and at least 1, 2, 3, 4, 5, 6, 7, 8, 9, 10, or more nucleotides on each side of positions comprising a guanine at a position corresponding to position 305 according to SEQ ID NO:13, or a guanine at a position corresponding to position 305 according to SEQ ID NO:25.

In some embodiments, to determine whether a GPAM nucleic acid molecule (mRNA molecule or cDNA molecule), or complement thereof, within a biological sample comprises a nucleotide sequence comprising a guanine at a position corresponding to position 170 according to SEQ ID NO:14 (mRNA molecule), or a guanine at a position corresponding to position 170 according to SEQ ID NO:26 (cDNA molecule), the biological sample can be subjected to an amplification method using a primer pair that includes a first primer derived from the 5′ flanking sequence adjacent to a guanine at a position corresponding to position 170 according to SEQ ID NO:14, or a guanine at a position corresponding to position 170 according to SEQ ID NO:26, and a second primer derived from the 3′ flanking sequence adjacent to a guanine at a position corresponding to position 170 according to SEQ ID NO:14, or a guanine at a position corresponding to position 170 according to SEQ ID NO:26 to produce an amplicon that is indicative of the presence of the SNP at positions encoding a guanine at a position corresponding to position 170 according to SEQ ID NO:14, or a guanine at a position corresponding to position 170 according to SEQ ID NO:26. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a guanine at a position corresponding to position 170 according to SEQ ID NO:14, or a guanine at a position corresponding to position 170 according to SEQ ID NO:26, and at least 1, 2, 3, 4, 5, 6, 7, 8, 9, 10, or more nucleotides on each side of positions comprising a guanine at a position corresponding to position 170 according to SEQ ID NO:14, or a guanine at a position corresponding to position 170 according to SEQ ID NO:26.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a human GPAM predicted loss-of-function polypeptide comprising performing an assay on a sample obtained from a subject to determine whether a GPAM polypeptide in the subject contains one or more variations that causes the polypeptide to have a loss-of-function (partial or complete) or predicted loss-of-function (partial or complete). The GPAM predicted loss-of-function polypeptide can be any of the GPAM truncated variant polypeptides described herein. In some embodiments, the methods detect the presence of GPAM Ile43Val.

In some embodiments, the methods comprise performing an assay on a sample obtained from a subject to determine whether a GPAM polypeptide in the sample comprises a valine at a position corresponding to position 43 according to SEQ ID NO:29. In some embodiments, the methods comprise performing an assay on a sample obtained from a subject to determine whether a GPAM polypeptide in the sample comprises a valine at a position corresponding to position 43 according to SEQ ID NO:30.

In some embodiments, the detecting step comprises sequencing at least a portion of the polypeptide that comprises a position corresponding to position 43 according to SEQ ID NO:29, SEQ ID NO:27, or SEQ ID NO:28. In some embodiments, the detecting step comprises sequencing at least a portion of the polypeptide that comprises a position corresponding to position 43 according to SEQ ID NO:30, SEQ ID NO:27, or SEQ ID NO:28.

In some embodiments, the detecting step comprises an immunoassay for detecting the presence of a polypeptide that comprises a position corresponding to position 43 according to SEQ ID NO:29, SEQ ID NO:27, or SEQ ID NO:28. In some embodiments, the detecting step comprises an immunoassay for detecting the presence of a polypeptide that comprises a position corresponding to position 43 according to SEQ ID NO:30, SEQ ID NO:27, or SEQ ID NO:28.

In some embodiments, when the subject does not have a GPAM predicted loss-of-function polypeptide, then the subject has an increased risk for developing liver disease. In some embodiments, when the subject has a GPAM predicted loss-of-function polypeptide, then the subject has a decreased risk for developing liver disease.

The present disclosure also provides isolated nucleic acid molecules that hybridize to GPAM variant genomic nucleic acid molecules, GPAM variant mRNA molecules, and/or GPAM variant cDNA molecules (such as any of the genomic variant nucleic acid molecules, mRNA variant molecules, and cDNA variant molecules disclosed herein). In some embodiments, the isolated nucleic acid molecules hybridize to a portion of the GPAM nucleic acid molecule that includes a position corresponding to: position 3,195 according to SEQ ID NO:2, position 327 according to SEQ ID NO:9, or position 327 according to SEQ ID NO:21. In some embodiments, the isolated nucleic acid molecules hybridize to a portion of the GPAM nucleic acid molecule that includes a position corresponding to: position 291 according to SEQ ID NO:10, or position 291 according to SEQ ID NO:22. In some embodiments, the isolated nucleic acid molecules hybridize to a portion of the GPAM nucleic acid molecule that includes a position corresponding to: position 323 according to SEQ ID NO:11, or position 323 according to SEQ ID NO:23. In some embodiments, the isolated nucleic acid molecules hybridize to a portion of the GPAM nucleic acid molecule that includes a position corresponding to: position 326 according to SEQ ID NO:12, or position 326 according to SEQ ID NO:24. In some embodiments, the isolated nucleic acid molecules hybridize to a portion of the GPAM nucleic acid molecule that includes a position corresponding to: position 305 according to SEQ ID NO:13, or position 305 according to SEQ ID NO:25. In some embodiments, the isolated nucleic acid molecules hybridize to a portion of the GPAM nucleic acid molecule that includes a position corresponding to: position 170 according to SEQ ID NO:14, or position 170 according to SEQ ID NO:26.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to GPAM variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to GPAM variant genomic nucleic acid molecules, GPAM variant mRNA molecules, and/or GPAM variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human GPAM polypeptide, wherein the portion comprises a position corresponding to: position 3,195 according to SEQ ID NO:2, or the complement thereof; position 327 according to SEQ ID NO:9, or the complement thereof; or position 327 according to SEQ ID NO:21, or the complement thereof. In some embodiments, the alteration-specific probe or alteration-specific primer comprises a nucleotide sequence which is complementary to a portion of a nucleotide sequence comprising positions corresponding to: positions 3,195-3,197 according to SEQ ID NO:2, or the complement thereof; positions 327-329 according to SEQ ID NO:9, or the complement thereof; and/or positions 327-329 according to SEQ ID NO:21, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human GPAM polypeptide, wherein the portion comprises a position corresponding to: position 291 according to SEQ ID NO:10, or the complement thereof; or position 291 according to SEQ ID NO:22, or the complement thereof. In some embodiments, the alteration-specific probe or alteration-specific primer comprises a nucleotide sequence which is complementary to a portion of a nucleotide sequence comprising positions corresponding to: positions 291-293 according to SEQ ID NO:10, or the complement thereof; and/or positions 291-293 according to SEQ ID NO:22, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human GPAM polypeptide, wherein the portion comprises a position corresponding to: position 323 according to SEQ ID NO:11, or the complement thereof; or position 323 according to SEQ ID NO:23, or the complement thereof. In some embodiments, the alteration-specific probe or alteration-specific primer comprises a nucleotide sequence which is complementary to a portion of a nucleotide sequence comprising positions corresponding to: positions 323-325 according to SEQ ID NO:11, or the complement thereof; and/or positions 323-325 according to SEQ ID NO:23, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human GPAM polypeptide, wherein the portion comprises a position corresponding to: position 326 according to SEQ ID NO:12, or the complement thereof; or position 326 according to SEQ ID NO:24, or the complement thereof. In some embodiments, the alteration-specific probe or alteration-specific primer comprises a nucleotide sequence which is complementary to a portion of a nucleotide sequence comprising positions corresponding to positions 326-328 according to SEQ ID NO:12, or the complement thereof; and/or positions 326-328 according to SEQ ID NO:24, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human GPAM polypeptide, wherein the portion comprises a position corresponding to: position 305 according to SEQ ID NO:13, or the complement thereof; or position 305 according to SEQ ID NO:25, or the complement thereof. In some embodiments, the alteration-specific probe or alteration-specific primer comprises a nucleotide sequence which is complementary to a portion of a nucleotide sequence comprising positions corresponding to: positions 305-307 according to SEQ ID NO:13, or the complement thereof; and/or positions 305-307 according to SEQ ID NO:25, or the complement thereof.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human GPAM polypeptide, wherein the portion comprises a position corresponding to: position 170 according to SEQ ID NO:14, or the complement thereof; or position 170 according to SEQ ID NO:26, or the complement thereof. In some embodiments, the alteration-specific probe or alteration-specific primer comprises a nucleotide sequence which is complementary to a portion of a nucleotide sequence comprising positions corresponding to: positions 170-172 according to SEQ ID NO:14, or the complement thereof; and/or positions 170-172 according to SEQ ID NO:26, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the GPAM variant genomic nucleic acid molecules, GPAM variant mRNA molecules, and/or GPAM variant cDNA molecules disclosed herein. The primers described herein can be used to amplify GPAM variant genomic nucleic acid molecules, GPAM variant mRNA molecules, or GPAM variant cDNA molecules, or a fragment thereof.

The present disclosure also provides pairs of primers comprising any of the primers described above. For example, if one of the primers' 3′-ends hybridizes to an adenine at a position corresponding to position 3,195 according to SEQ ID NO:1 (rather than guanine) in a particular GPAM nucleic acid molecule, then the presence of the amplified fragment would indicate the presence of a GPAM reference genomic nucleic acid molecule. Conversely, if one of the primers' 3′-ends hybridizes to a guanine at a position corresponding to position 3,195 according to SEQ ID NO:2 (rather than adenine) in a particular GPAM nucleic acid molecule, then the presence of the amplified fragment would indicate the presence of the GPAM variant genomic nucleic acid molecule. In some embodiments, the nucleotide of the primer complementary to the guanine at a position corresponding to position 3,195 according to SEQ ID NO:2 can be at the 3′ end of the primer. In addition, if one of the primers' 3′-ends hybridizes to an adenine at a position corresponding to position 327 according to SEQ ID NO:3 (rather than guanine) in a particular GPAM nucleic acid molecule, then the presence of the amplified fragment would indicate the presence of a GPAM reference mRNA molecule. Conversely, if one of the primers' 3′-ends hybridizes to a guanine at a position corresponding to position 327 according to SEQ ID NO:9 (rather than adenine) in a particular GPAM mRNA molecule, then the presence of the amplified fragment would indicate the presence of the GPAM variant mRNA molecule. In some embodiments, the nucleotide of the primer complementary to the guanine at a position corresponding to position 327 according to SEQ ID NO:9 can be at the 3′ end of the primer. In addition, if one of the primers' 3′-ends hybridizes to an adenine at a position corresponding to position 327 according to SEQ ID NO:15 (rather than guanine) in a particular GPAM nucleic acid molecule, then the presence of the amplified fragment would indicate the presence of a GPAM reference cDNA molecule. Conversely, if one of the primers' 3′-ends hybridizes to a guanine at a position corresponding to position 327 according to SEQ ID NO:21 (rather than adenine) in a particular GPAM cDNA molecule, then the presence of the amplified fragment would indicate the presence of the GPAM variant cDNA molecule. In some embodiments, the nucleotide of the primer complementary to the guanine at a position corresponding to position 327 according to SEQ ID NO:21 can be at the 3′ end of the primer.

In addition, if one of the primers' 3′-ends hybridizes to an adenine at a position corresponding to position 291 according to SEQ ID NO:4 (rather than guanine) in a particular GPAM nucleic acid molecule, then the presence of the amplified fragment would indicate the presence of a GPAM reference mRNA molecule. Conversely, if one of the primers' 3′-ends hybridizes to a guanine at a position corresponding to position 291 according to SEQ ID NO:10 (rather than adenine) in a particular GPAM mRNA molecule, then the presence of the amplified fragment would indicate the presence of the GPAM variant mRNA molecule. In some embodiments, the nucleotide of the primer complementary to the guanine at a position corresponding to position 291 according to SEQ ID NO:10 can be at the 3′ end of the primer. In addition, if one of the primers' 3′-ends hybridizes to an adenine at a position corresponding to position 291 according to SEQ ID NO:16 (rather than guanine) in a particular GPAM nucleic acid molecule, then the presence of the amplified fragment would indicate the presence of a GPAM reference cDNA molecule. Conversely, if one of the primers' 3′-ends hybridizes to a guanine at a position corresponding to position 291 according to SEQ ID NO:22 (rather than adenine) in a particular GPAM cDNA molecule, then the presence of the amplified fragment would indicate the presence of the GPAM variant cDNA molecule. In some embodiments, the nucleotide of the primer complementary to the guanine at a position corresponding to position 291 according to SEQ ID NO:22 can be at the 3′ end of the primer.

In addition, if one of the primers' 3′-ends hybridizes to an adenine at a position corresponding to position 323 according to SEQ ID NO:5 (rather than guanine) in a particular GPAM nucleic acid molecule, then the presence of the amplified fragment would indicate the presence of a GPAM reference mRNA molecule. Conversely, if one of the primers' 3′-ends hybridizes to a guanine at a position corresponding to position 323 according to SEQ ID NO:11 (rather than adenine) in a particular GPAM mRNA molecule, then the presence of the amplified fragment would indicate the presence of the GPAM variant mRNA molecule. In some embodiments, the nucleotide of the primer complementary to the guanine at a position corresponding to position 323 according to SEQ ID NO:11 can be at the 3′ end of the primer. In addition, if one of the primers' 3′-ends hybridizes to an adenine at a position corresponding to position 323 according to SEQ ID NO:17 (rather than guanine) in a particular GPAM nucleic acid molecule, then the presence of the amplified fragment would indicate the presence of a GPAM reference cDNA molecule. Conversely, if one of the primers' 3′-ends hybridizes to a guanine at a position corresponding to position 323 according to SEQ ID NO:23 (rather than adenine) in a particular GPAM cDNA molecule, then the presence of the amplified fragment would indicate the presence of the GPAM variant cDNA molecule. In some embodiments, the nucleotide of the primer complementary to the guanine at a position corresponding to position 323 according to SEQ ID NO:23 can be at the 3′ end of the primer.

In addition, if one of the primers' 3′-ends hybridizes to an adenine at a position corresponding to position 326 according to SEQ ID NO:6 (rather than guanine) in a particular GPAM nucleic acid molecule, then the presence of the amplified fragment would indicate the presence of a GPAM reference mRNA molecule. Conversely, if one of the primers' 3′-ends hybridizes to a guanine at a position corresponding to position 326 according to SEQ ID NO:12 (rather than adenine) in a particular GPAM mRNA molecule, then the presence of the amplified fragment would indicate the presence of the GPAM variant mRNA molecule. In some embodiments, the nucleotide of the primer complementary to the guanine at a position corresponding to position 326 according to SEQ ID NO:12 can be at the 3′ end of the primer. In addition, if one of the primers' 3′-ends hybridizes to an adenine at a position corresponding to position 326 according to SEQ ID NO:18 (rather than guanine) in a particular GPAM nucleic acid molecule, then the presence of the amplified fragment would indicate the presence of a GPAM reference cDNA molecule. Conversely, if one of the primers' 3′-ends hybridizes to a guanine at a position corresponding to position 326 according to SEQ ID NO:24 (rather than adenine) in a particular GPAM cDNA molecule, then the presence of the amplified fragment would indicate the presence of the GPAM variant cDNA molecule. In some embodiments, the nucleotide of the primer complementary to the guanine at a position corresponding to position 326 according to SEQ ID NO:24 can be at the 3′ end of the primer.

In addition, if one of the primers' 3′-ends hybridizes to an adenine at a position corresponding to position 305 according to SEQ ID NO:7 (rather than guanine) in a particular GPAM nucleic acid molecule, then the presence of the amplified fragment would indicate the presence of a GPAM reference mRNA molecule. Conversely, if one of the primers' 3′-ends hybridizes to a guanine at a position corresponding to position 305 according to SEQ ID NO:13 (rather than adenine) in a particular GPAM mRNA molecule, then the presence of the amplified fragment would indicate the presence of the GPAM variant mRNA molecule. In some embodiments, the nucleotide of the primer complementary to the guanine at a position corresponding to position 305 according to SEQ ID NO:13 can be at the 3′ end of the primer. In addition, if one of the primers' 3′-ends hybridizes to an adenine at a position corresponding to position 305 according to SEQ ID NO:19 (rather than guanine) in a particular GPAM nucleic acid molecule, then the presence of the amplified fragment would indicate the presence of a GPAM reference cDNA molecule. Conversely, if one of the primers' 3′-ends hybridizes to a guanine at a position corresponding to position 305 according to SEQ ID NO:25 (rather than adenine) in a particular GPAM cDNA molecule, then the presence of the amplified fragment would indicate the presence of the GPAM variant cDNA molecule. In some embodiments, the nucleotide of the primer complementary to the guanine at a position corresponding to position 305 according to SEQ ID NO:25 can be at the 3′ end of the primer.

In addition, if one of the primers' 3′-ends hybridizes to an adenine at a position corresponding to position 170 according to SEQ ID NO:8 (rather than guanine) in a particular GPAM nucleic acid molecule, then the presence of the amplified fragment would indicate the presence of a GPAM reference mRNA molecule. Conversely, if one of the primers' 3′-ends hybridizes to a guanine at a position corresponding to position 170 according to SEQ ID NO:14 (rather than adenine) in a particular GPAM mRNA molecule, then the presence of the amplified fragment would indicate the presence of the GPAM variant mRNA molecule. In some embodiments, the nucleotide of the primer complementary to the guanine at a position corresponding to position 170 according to SEQ ID NO:14 can be at the 3′ end of the primer. In addition, if one of the primers' 3′-ends hybridizes to an adenine at a position corresponding to position 170 according to SEQ ID NO:20 (rather than guanine) in a particular GPAM nucleic acid molecule, then the presence of the amplified fragment would indicate the presence of a GPAM reference cDNA molecule. Conversely, if one of the primers' 3′-ends hybridizes to a guanine at a position corresponding to position 170 according to SEQ ID NO:26 (rather than adenine) in a particular GPAM cDNA molecule, then the presence of the amplified fragment would indicate the presence of the GPAM variant cDNA molecule. In some embodiments, the nucleotide of the primer complementary to the guanine at a position corresponding to position 170 according to SEQ ID NO:26 can be at the 3′ end of the primer.

In the context of the disclosure “specifically hybridizes” means that the probe or primer (such as, for example, the alteration-specific probe or alteration-specific primer) does not hybridize to a nucleic acid sequence encoding a GPAM reference genomic nucleic acid molecule, a GPAM reference mRNA molecule, and/or a GPAM reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of a GPAM reference genomic nucleic acid molecule is set forth in SEQ ID NO:1. Referring to SEQ ID NO:1, position 3,195 is an adenine.

A variant genomic nucleic acid molecule of GPAM exists, wherein the adenine at position 3,195 is replaced with a guanine. The nucleotide sequence of this GPAM variant genomic nucleic acid molecule is set forth in SEQ ID NO:2.

The nucleotide sequence of a GPAM reference mRNA molecule is set forth in SEQ ID NO:3. Referring to SEQ ID NO:3, position 327 is an adenine. The nucleotide sequence of another GPAM reference mRNA molecule is set forth in SEQ ID NO:4. Referring to SEQ ID NO:4, position 291 is an adenine. The nucleotide sequence of another GPAM reference mRNA molecule is set forth in SEQ ID NO:5. Referring to SEQ ID NO:5, position 323 is an adenine. The nucleotide sequence of another GPAM reference mRNA molecule is set forth in SEQ ID NO:6. Referring to SEQ ID NO:6, position 326 is an adenine. The nucleotide sequence of another GPAM reference mRNA molecule is set forth in SEQ ID NO:7. Referring to SEQ ID NO:7, position 305 is an adenine. The nucleotide sequence of another GPAM reference mRNA molecule is set forth in SEQ ID NO:8. Referring to SEQ ID NO:8, position 170 is an adenine.

A variant mRNA molecule of GPAM exists, wherein the adenine at position 327 is replaced with guanine. The nucleotide sequence of this GPAM variant mRNA molecule is set forth in SEQ ID NO:9.

Another variant mRNA molecule of GPAM exists, wherein the adenine at position 291 is replaced with guanine. The nucleotide sequence of this GPAM variant mRNA molecule is set forth in SEQ ID NO:10.

Another variant mRNA molecule of GPAM exists, wherein the adenine at position 323 is replaced with guanine. The nucleotide sequence of this GPAM variant mRNA molecule is set forth in SEQ ID NO:11.

Another variant mRNA molecule of GPAM exists, wherein the adenine at position 326 is replaced with guanine. The nucleotide sequence of this GPAM variant mRNA molecule is set forth in SEQ ID NO:12.

Another variant mRNA molecule of GPAM exists, wherein the adenine at position 305 is replaced with guanine. The nucleotide sequence of this GPAM variant mRNA molecule is set forth in SEQ ID NO:13.

Another variant mRNA molecule of GPAM exists, wherein the adenine at position 170 is replaced with guanine. The nucleotide sequence of this GPAM variant mRNA molecule is set forth in SEQ ID NO:14.

The nucleotide sequence of a GPAM reference cDNA molecule is set forth in SEQ ID NO:15. Referring to SEQ ID NO:15, position 327 is an adenine. The nucleotide sequence of another GPAM reference cDNA molecule is set forth in SEQ ID NO:16. Referring to SEQ ID NO:16, position 291 is an adenine. The nucleotide sequence of another GPAM reference cDNA molecule is set forth in SEQ ID NO:17. Referring to SEQ ID NO:17, position 323 is an adenine. The nucleotide sequence of another GPAM reference cDNA molecule is set forth in SEQ ID NO:18. Referring to SEQ ID NO:18, position 326 is an adenine. The nucleotide sequence of another GPAM reference cDNA molecule is set forth in SEQ ID NO:19. Referring to SEQ ID NO:19, position 305 is an adenine. The nucleotide sequence of another GPAM reference cDNA molecule is set forth in SEQ ID NO:20. Referring to SEQ ID NO:20, position 170 is an adenine.

A variant cDNA molecule of GPAM exists, wherein the adenine at position 327 is replaced with guanine. The nucleotide sequence of this GPAM variant cDNA molecule is set forth in SEQ ID NO:21.

Another variant cDNA molecule of GPAM exists, wherein the adenine at position 291 is replaced with guanine. The nucleotide sequence of this GPAM variant cDNA molecule is set forth in SEQ ID NO:22.

Another variant cDNA molecule of GPAM exists, wherein the adenine at position 323 is replaced with guanine. The nucleotide sequence of this GPAM variant cDNA molecule is set forth in SEQ ID NO:23.

Another variant cDNA molecule of GPAM exists, wherein the adenine at position 326 is replaced with guanine. The nucleotide sequence of this GPAM variant cDNA molecule is set forth in SEQ ID NO:24.

Another variant cDNA molecule of GPAM exists, wherein the adenine at position 305 is replaced with guanine. The nucleotide sequence of this GPAM variant cDNA molecule is set forth in SEQ ID NO:25.

Another variant cDNA molecule of GPAM exists, wherein the adenine at position 170 is replaced with guanine. The nucleotide sequence of this GPAM variant cDNA molecule is set forth in SEQ ID NO:26.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:3, or SEQ ID NO:15). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a nucleic acid molecule comprising a nucleotide sequence encoding a human GPAM polypeptide, wherein the nucleotide sequence comprises a guanine at a position corresponding to position 3,195 according to SEQ ID NO:2 means that if the nucleotide sequence of the GPAM genomic nucleic acid molecule is aligned to the sequence of SEQ ID NO:2, the GPAM sequence has a guanine residue at the position that corresponds to position 3,195 of SEQ ID NO:2. The same applies for mRNA molecules comprising a nucleotide sequence encoding a human GPAM polypeptide, wherein the nucleotide sequence comprises a guanine at a position corresponding to position 327 according to SEQ ID NO:9, and cDNA molecules comprising a nucleotide sequence encoding a human GPAM polypeptide, wherein the nucleotide sequence comprises a guanine at a position corresponding to position 327 according to SEQ ID NO:21. In other words, these phrases refer to a nucleic acid molecule encoding a GPAM polypeptide, wherein the genomic nucleic acid molecule has a nucleotide sequence that comprises a guanine residue that is homologous to the guanine residue at position 3,195 of SEQ ID NO:2 (or wherein the mRNA molecule has a nucleotide sequence that comprises a guanine residue that is homologous to the guanine residue at position 327 of SEQ ID NO:9, or wherein the cDNA molecule has a nucleotide sequence that comprises a guanine residue that is homologous to the guanine residue at position 327 of SEQ ID NO:21). Herein, such a sequence is also referred to as “GPAM sequence with the Ile43Val alteration” or “GPAM sequence with the Ile43Val variation” referring to genomic nucleic acid molecules (or “GPAM sequence with the A327G alteration” or “GPAM sequence with the A327G variation” referring to mRNA molecules, and “GPAM sequence with the A327G alteration” or “GPAM sequence with the A327G variation” referring to cDNA molecules).

As described herein, a position within a GPAM genomic nucleic acid molecule that corresponds to position 3,195 according to SEQ ID NO:2, for example, can be identified by performing a sequence alignment between the nucleotide sequence of a particular GPAM nucleic acid molecule and the nucleotide sequence of SEQ ID NO:2. A variety of computational algorithms exist that can be used for performing a sequence alignment to identify a nucleotide position that corresponds to, for example, position 3,195 in SEQ ID NO:2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of a GPAM reference polypeptide is set forth in SEQ ID NO:27. Referring to SEQ ID NO:27, the GPAM reference polypeptide is 828 amino acids in length. Referring to SEQ ID NO:27, position 43 is isoleucine. The amino acid sequence of another GPAM reference polypeptide is set forth in SEQ ID NO:28. Referring to SEQ ID NO:28, the GPAM reference polypeptide is 710 amino acids in length. Referring to SEQ ID NO:28, position 43 is isoleucine.

A GPAM variant polypeptide exists, the amino acid sequence of which is set forth in SEQ ID NO:29. Referring to SEQ ID NO:29, the GPAM variant polypeptide is 828 amino acids in length. Referring to SEQ ID NO:29, position 43 is valine.

Another GPAM variant polypeptide exists, the amino acid sequence of which is set forth in SEQ ID NO:30. Referring to SEQ ID NO:30, the GPAM variant polypeptide is 710 amino acids in length. Referring to SEQ ID NO:30, position 43 is valine.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit liver disease for use in the treatment of liver disease (or for use in the preparation of a medicament for treating liver disease) in a subject, wherein the subject has any of the genomic nucleic acid molecules, mRNA molecules, and/or cDNA molecules encoding a human GPAM polypeptide described herein. The therapeutic agents that treat or inhibit liver disease can be any of the therapeutic agents that treat or inhibit liver disease described herein.

In some embodiments, the subject comprises: a genomic nucleic acid molecule having a nucleotide sequence encoding a human GPAM polypeptide, wherein the nucleotide sequence comprises a guanine at a position corresponding to position 3,195 according to SEQ ID NO:2, or the complement thereof; an mRNA molecule having a nucleotide sequence encoding a human GPAM polypeptide, wherein the nucleotide sequence comprises a guanine at a position corresponding to position 327 according to SEQ ID NO:9, or the complement thereof; a cDNA molecule having a nucleotide sequence encoding a human GPAM polypeptide, wherein the nucleotide sequence comprises a guanine at a position corresponding to position 327 according to SEQ ID NO:21, or the complement thereof; or a GPAM polypeptide that comprises a valine at a position corresponding to position 43 according to SEQ ID NO:29.

In some embodiments, the subject comprises: an mRNA molecule having a nucleotide sequence encoding a human GPAM polypeptide, wherein the nucleotide sequence comprises a guanine at a position corresponding to position 291 according to SEQ ID NO:10, or the complement thereof; a cDNA molecule having a nucleotide sequence encoding a human GPAM polypeptide, wherein the nucleotide sequence comprises a guanine at a position corresponding to position 291 according to SEQ ID NO:22, or the complement thereof; or a GPAM polypeptide that comprises a valine at a position corresponding to position 43 according to SEQ ID NO:30.

In some embodiments, the subject comprises: an mRNA molecule having a nucleotide sequence encoding a human GPAM polypeptide, wherein the nucleotide sequence comprises a guanine at a position corresponding to position 323 according to SEQ ID NO:11, or the complement thereof; or a cDNA molecule having a nucleotide sequence encoding a human GPAM polypeptide, wherein the nucleotide sequence comprises a guanine at a position corresponding to position 323 according to SEQ ID NO:23, or the complement thereof.

In some embodiments, the subject comprises: an mRNA molecule having a nucleotide sequence encoding a human GPAM polypeptide, wherein the nucleotide sequence comprises a guanine at a position corresponding to position 326 according to SEQ ID NO:12, or the complement thereof; or a cDNA molecule having a nucleotide sequence encoding a human GPAM polypeptide, wherein the nucleotide sequence comprises a guanine at a position corresponding to position 326 according to SEQ ID NO:24, or the complement thereof.

In some embodiments, the subject comprises: an mRNA molecule having a nucleotide sequence encoding a human GPAM polypeptide, wherein the nucleotide sequence comprises a guanine at a position corresponding to position 305 according to SEQ ID NO:13, or the complement thereof; a or cDNA molecule having a nucleotide sequence encoding a human GPAM polypeptide, wherein the nucleotide sequence comprises a guanine at a position corresponding to position 305 according to SEQ ID NO:25, or the complement thereof.

In some embodiments, the subject comprises: an mRNA molecule having a nucleotide sequence encoding a human GPAM polypeptide, wherein the nucleotide sequence comprises a guanine at a position corresponding to position 170 according to SEQ ID NO:14, or the complement thereof; or a cDNA molecule having a nucleotide sequence encoding a human GPAM polypeptide, wherein the nucleotide sequence comprises a guanine at a position corresponding to position 170 according to SEQ ID NO:26, or the complement thereof.

The present disclosure also provides GPAM inhibitors for use in the treatment of liver disease (or for use in the preparation of a medicament for treating liver disease) in a subject, wherein the subject has any of the genomic nucleic acid molecules, mRNA molecules, and/or cDNA molecules encoding a human GPAM polypeptide described herein. The GPAM inhibitors can be any of the GPAM inhibitors described herein.

In some embodiments, the subject comprises: a genomic nucleic acid molecule having a nucleotide sequence encoding a human GPAM polypeptide, wherein the nucleotide sequence comprises a guanine at a position corresponding to position 3,195 according to SEQ ID NO:2, or the complement thereof; an mRNA molecule having a nucleotide sequence encoding a human GPAM polypeptide, wherein the nucleotide sequence comprises a guanine at a position corresponding to position 327 according to SEQ ID NO:9, or the complement thereof; a cDNA molecule having a nucleotide sequence encoding a human GPAM polypeptide, wherein the nucleotide sequence comprises a guanine at a position corresponding to position 327 according to SEQ ID NO:21, or the complement thereof; or a GPAM polypeptide that comprises a valine at a position corresponding to position 43 according to SEQ ID NO:29. The GPAM inhibitors can be any of the GPAM inhibitors described herein.

In some embodiments, the subject comprises: an mRNA molecule having a nucleotide sequence encoding a human GPAM polypeptide, wherein the nucleotide sequence comprises a guanine at a position corresponding to position 291 according to SEQ ID NO:10, or the complement thereof; a cDNA molecule having a nucleotide sequence encoding a human GPAM polypeptide, wherein the nucleotide sequence comprises a guanine at a position corresponding to position 291 according to SEQ ID NO:22, or the complement thereof; or a GPAM polypeptide that comprises a valine at a position corresponding to position 43 according to SEQ ID NO:30.

In some embodiments, the subject comprises: an mRNA molecule having a nucleotide sequence encoding a human GPAM polypeptide, wherein the nucleotide sequence comprises a guanine at a position corresponding to position 323 according to SEQ ID NO:11, or the complement thereof; or a cDNA molecule having a nucleotide sequence encoding a human GPAM polypeptide, wherein the nucleotide sequence comprises a guanine at a position corresponding to position 323 according to SEQ ID NO:23, or the complement thereof.

In some embodiments, the subject comprises: an mRNA molecule having a nucleotide sequence encoding a human GPAM polypeptide, wherein the nucleotide sequence comprises a guanine at a position corresponding to position 326 according to SEQ ID NO:12, or the complement thereof; or a cDNA molecule having a nucleotide sequence encoding a human GPAM polypeptide, wherein the nucleotide sequence comprises a guanine at a position corresponding to position 326 according to SEQ ID NO:24, or the complement thereof.

In some embodiments, the subject comprises: an mRNA molecule having a nucleotide sequence encoding a human GPAM polypeptide, wherein the nucleotide sequence comprises a guanine at a position corresponding to position 305 according to SEQ ID NO:13, or the complement thereof; or a cDNA molecule having a nucleotide sequence encoding a human GPAM polypeptide, wherein the nucleotide sequence comprises a guanine at a position corresponding to position 305 according to SEQ ID NO:25, or the complement thereof.

In some embodiments, the subject comprises: an mRNA molecule having a nucleotide sequence encoding a human GPAM polypeptide, wherein the nucleotide sequence comprises a guanine at a position corresponding to position 170 according to SEQ ID NO:14, or the complement thereof; or a cDNA molecule having a nucleotide sequence encoding a human GPAM polypeptide, wherein the nucleotide sequence comprises a guanine at a position corresponding to position 170 according to SEQ ID NO:26, or the complement thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Loss of Function of the Gene Encoding Glycerol-3-Phosphate Acyltransferase, Mitochondrial (GPAM) is Associated with Lower Liver Fat, Lower ALT, and Protection Against Liver Disease

To identify genetic factors contributing to chronic liver disease, imputed genotype data, exome sequence data, and magnetic resonance imaging (MRI) derived phenotypes were analyzed on 40,058 participants of the UK Biobank cohort (UKB). Statistically significant findings were subsequently evaluated for their relationship with liver injury as measured by ALT, a widely used measure of liver damage. Associations with metabolic phenotypes, diagnosis of chronic liver disease, type 2 diabetes, and coronary artery disease were also estimated. This analysis included 597,856 participants of European ancestry in the UK Biobank cohort (UKB), the Geisinger Health System MyCode Community Health Initiative cohort study (GHS) and Mount Sinai's BioMe Personalized Medicine Cohort (SINAI).

Genome wide analysis studies (GWAS) of MRI derived phenotypes were carried out using an imputed dataset of 11,914,698 variants and 37,250 individuals of European ancestry participating in UKB. Outcomes phenotypes included proton density fat fraction (PDFF), a measure of hepatic fat content, extracellular fluid fraction (ECF), a proxy for liver fibrosis and inflammation and iron corrected T1 measurements (cT1). PDFF is defined as the ratio of density of mobile protons from fat (triglycerides) and the total density of protons from mobile triglycerides and mobile water and reflects the concentration of fat within a tissue. ECF and cT1 measure the increases in extracellular tissue fluids that occur in response to inflammation and fibrosis.

The GWAS for PDFF identified 19 associations at the genome-wide level of statistical significance (p<5×10⁻⁸). One locus comprised rs2792751 (T->C, alternative allele frequency of 73%), encoding for a common missense variation changing an isoleucine into a valine at the 43rd amino acid of the encoded GPAM protein (Ile43Val). Ile43Val was in near perfect linkage disequilibrium (r²=0.99) with the sentinel genetic variant (i.e., the variant with the lowest p-value for association) for PDFF at the GPAM locus in the European population. Ile43Val was significantly associated with lower PDFF levels at P<6.9×10⁻¹⁶ as shown in Table 2.

TABLE 2
Association of Ile43Val and GPAM pLOF burden with liver fat content as measured by magnetic
resonance imaging (MRI) derived proton density fat fraction (PDFF) in the UKB

| Genetic | | Per allele beta | | Genotype counts, | AAF, |
|---|---|---|---|---|---|
| exposure | Outcome | (95% confidence interval) | P-value | RR\|RA\|AA genotypes | fraction of 1 |
| p.Ile43Val | PDFF | −0.05 (−0.06, −0.04) SD | 6.9E−16 | 2,849\|14,700\|19,701 | 0.73 |
| pLOF; AAF < 1% | PDFF | −0.34 (−0.64, −0.04) SD | 0.024 | 35,252\|22\|0 | 0.0003 |

Note:
PDFF, proton density fat fraction; RR, number of individuals carrying no alternative variant allele (homozygous non-carriers); RA, number of individuals carrying a single alternative variant allele (heterozygous carriers); AA, number of individuals carrying alternative variant alleles in both copies of the gene (homozygous carriers); The alternative allele is the allele causing loss of function or change in amino acid following human genome sequence reference build 38 and HGVS protein sequence nomenclature; AAF, the alternative allele frequency; SD, standard deviation units.

Using exome sequence data, the association for the burden of rare (AAF<1%) predicted loss-of-function (pLOF) variants in GPAM gene with liver fat as measured by PDFF at liver MRI was estimated. In this analysis, pLOF variants in GPAM were associated with lower PDFF levels at p=0.024 as shown in Table 2.

A meta-analysis of genetic association studies was then carried out to identify genes associated with ALT, a biomarker of liver damage. The Ile43Val variant at the GPAM locus was found to be associated with lower ALT levels at P<4.20×10⁻⁵⁴ as shown in Table 3 and to be in perfect linkage disequilibrium (r2>0.99) with the sentinel variant at this locus.

TABLE 3
The GPAM Ile43Val allele and the burden of protein-truncating variants in GPAM are
associated with lower circulating ALT levels. Results are shown both in units of
standard deviation, and in the original clinical units (international units/liter)

| Genetic | | Per allele beta | | | Genotype counts, | AAF, |
|---|---|---|---|---|---|---|
| exposure | Outcome | (95% confidence interval) | | P-value | RR\|RA\|AA genotypes | fraction of 1 |
| p.Ile43Val | ALT | −0.03 | (−0.04, −0.03) SD | 2.7E−55 | 43,024\|220,632\|287,173 | 0.72 |
| | | −0.41 | (−0.55, −0.41) U/L | | | |
| pLOF; AAF < 1% | ALT | −0.27 | (−0.38, −0.16) SD | 2.50E−06 | 518,035\|276\|0 | 0.00027 |
| | | −3.69 | (−5.19, −2.19) U/L | | | |

Note:
RR, number of individuals carrying no alternative variant allele (homozygous non-carriers); RA, number of individuals carrying a single alternative variant allele (heterozygous carriers); AA, number of individuals carrying alternative variant alleles in both copies of the gene (homozygous carriers); the alternative allele is the allele causing loss of function or change in amino acid following human genome sequence reference build 38 and HGVS protein sequence nomenclature; AAF, alternative allele frequency; SD, standard deviation units; U/L, units per liter.

Using exome sequence data, the association for the burden of rare (AAF<1%) predicted loss-of-function (pLOF) variants in GPAM gene with ALT was estimated. In this analysis, pLOF variants in GPAM were strongly associated with reduced circulating ALT levels at p=2.5×10⁻⁰⁶ as shown in Table 3. The patterns of association described in Table 2 and Table 3 of Ile43Val and the GPAM pLOF burden indicates that Ile43Val is likely to cause loss of function of GPAM.

Table 4 shows that Ile43Val is associated with reduced measures of liver inflammation as measured on the MRI in UKB. These results, together with results in Table 2 and Table 3 indicate that loss of function of GPAM is associated with lower liver inflammation. Carriers of GPAM pLOF variants had numerically lower levels of liver inflammation at imaging, but the statistical power is limited to detect significant associations between the GPAM pLOF burden and liver inflammation based on the observed effects for Ile43Val and low frequency of GPAM pLOF variants.

TABLE 4
Significant association between Ile43Val and liver imaging
phenotypes in the UKB indicating liver inflammation

| Genetic | | Per allele beta | | | Genotype counts, | |
|---|---|---|---|---|---|---|
| exposure | Outcome | (95% confidence interval) | | P-value | RR\|RA\|AA genotypes | AAF |
| p.Ile43Val | cT1 | −0.03 | (−0.05, −0.02) SD | 3.00E−05 | 2,981\|15,324\|20,540 | 0.73 |
| p.Ile43Val | cT1* | −0.03 | (−0.05, −0.02) SD | 1.50E−06 | 2,849\|14,700\|19,701 | 0.73 |
| p.Ile43Val | ECF | −0.03 | (−0.05, −0.02) SD | 3.20E−05 | 2,981\|15,324\|20,540 | 0.73 |
| p.Ile43Val | ECF* | −0.03 | (−0.05, −0.02) SD | 1.80E−06 | 2,849\|14,700\|19,701 | 0.73 |
| pLOF; AAF < 1% | cT1 | −0.36 | (−0.76, 0.03) SD | 0.073 | 36,738\|22\|0 | 0.0003 |
| pLOF; AAF < 1% | cT1* | −0.28 | (−0.65, 0.08) SD | 0.13 | 35,252\|22\|0 | 0.00031 |
| pLOF; AAF < 1% | ECF | −0.36 | (−0.75, 0.04) SD | 0.076 | 36,738\|22\|0 | 0.0003 |
| pLOF; AAF < 1% | ECF* | −0.28 | (−0.64, 0.09) SD | 0.14 | 35,252\|22\|0 | 0.00031 |

Note:
*indicates that the phenotype was adjusted for technical covariates including BMI, alcohol usage, and diabetes; ECF indicates extracellular fluid; T1 indicates the time constant for recovery of longitudinal magnetization, it is a measure of how quickly the net magnetization recovers to its ground state, which is influenced by the strength of the magnetic field and tissue composition such as fat and/or iron in the tissue; cT1 = T1 corrected for the effects of liver iron content which result in T1 values being underestimated. RR, number of individuals carrying no alternative variant allele (homozygous non-carriers); RA, number of individuals carrying a single alternative variant allele (heterozygous carriers); AA, number of individuals carrying alternative variant alleles in both copies of the gene (homozygous carriers); The alternative allele is the allele causing loss of function or change in amino acid following human genome sequence reference build 38 and HGVS protein sequence nomenclature; AAF indicates the alternative allele frequency; SD indicates standard deviation units.

Table 5 and Table 6 shows associations between Ile43Val and multiple cardio-metabolic phenotypes, including lower HbA1c, lower apolipoprotein B, lower LDL-C, lower waist-hip ratio (adjust for BMI, WHRadjBMI), lower blood pressure, higher body mass index (BMI) and higher triglycerides. The variant was not associated with risk of type 2 diabetes or coronary artery disease.

TABLE 5
The Ile43Val variant in GPAM is associated with cardiovascular
and metabolic continuous phenotypes. The results are from an inverse
variance weighted meta-analysis in the GHS and UKB cohorts

| | Per allele Effect | | | Genotype counts, | |
|---|---|---|---|---|---|
| Outcome | (95% confidence interval) | | P-value | RR\|RA\|AA genotypes | AAF |
| LDL-C | −0.02 | (−0.03, −0.02) SD | 3.30E−36 | 41,427\|213,197\|277,973 | 0.72 |
| HDL-C | −0.04 | (−0.04, −0.04) SD | 6.63E−84 | 38,698\|198,885\|259,316 | 0.72 |
| Triglycerides | 0.02 | (0.01, 0.02) SD | 3.40E−20 | 41,539\|213,753\|278,608 | 0.72 |
| SBP | −0.01 | (−0.01, −0.00) SD | 7.30E−05 | 43,830\|224,050\|290,153 | 0.72 |
| DBP | −0.01 | (−0.01, −0.00) SD | 0.0029 | 43,553\|222,394\|287,977 | 0.72 |
| BMI | 0.01 | (0.01, 0.01) SD | 2.90E−06 | 43,185\|220,899\|286,632 | 0.72 |
| WHRadjBMI | −0.01 | (−0.01, −0.00) SD | 1.60E−05 | 65,480\|341,623\|450,945 | 0.72 |
| Glucose | 0.00 | (−0.00, 0.01) SD | 0.20 | 38,189\|196,338\|256,154 | 0.72 |
| HbA1c | 0.01 | (0.00, 0.01) SD | 0.0015 | 39,447\|203,863\|266,500 | 0.72 |

Note:
LDL-C indicates low-density lipoprotein cholesterol, HDL-C indicates high-density lipoprotein cholesterol, SBP indicates systolic blood pressure, DBP indicates diastolic blood pressure, BMI indicates body mass index, WHRadjBMI indicates waist hip ratio adjusted BMI, HbA1c indicates hemoglobin A1c, CAD indicates coronary artery disease; RR, number of individuals carrying no alternative variant allele (homozygous non-carriers); RA, number of individuals carrying a single alternative variant allele (heterozygous carriers); AA, number of individuals carrying alternative variant alleles in both copies of the gene (homozygous carriers); The alternative allele is the allele causing loss of function or change in amino acid following human genome sequence reference build 38 and HGVS protein sequence nomenclature; AAF indicates the alternative allele frequency; SD indicates standard deviation units.

TABLE 6
The variant Ile43Val in GPAM is not associated with risk of type
2 diabetes or coronary artery disease. The results are from an inverse
variance weighted meta-analysis in the GHS and UKB cohorts

| | Per allele odds ratio | | Genotype counts, | |
|---|---|---|---|---|
| | (95% CI) | | RR\|RA\|AA genotypes | AAF, |
| Outcome | for the outcome | p-value | in the case and control groups | fraction of 1 |
| Type 2 | 1.01 (0.99, 1.02) OR | 0.26 | Cases: 4,245\|21,062\|27,191 | 0.72 |
| diabetes | | | Controls: 38,531\|199,011\|259,679 | |
| CAD | 0.99 (0.98, 1.01) OR | 0.24 | Cases: 4,977\|25,413\|32,086 | 0.72 |
| | | | Controls: 25,319\|128,167\|165,470 | |

Note:
CAD indicates coronary artery disease; RR, number of individuals carrying no alternative variant allele (homozygous non-carriers); RA, number of individuals carrying a single alternative variant allele (heterozygous carriers); AA, number of individuals carrying alternative variant alleles in both copies of the gene (homozygous carriers); The alternative allele is the allele causing loss of function or change in amino acid following human genome sequence reference build 38 and HGVS protein sequence nomenclature; AAF indicates the alternative allele frequency; SD indicates standard deviation units.

Table 7 shows that Ile43Val in GPAM is also strongly associated with protection against liver diseases diagnoses, including parenchymal liver disease, alcoholic, non-alcoholic liver disease, liver fibrosis, cirrhosis of the liver and viral hepatitis. These results, together with the results in Table 2 and Table 3, indicate that loss-of-function of GPAM protects against various chronic liver diseases and viral hepatitis.

TABLE 7
Association of Ile43Val with clinical diagnoses of liver
disease in a meta-analysis of the UKB, GHS and SINAI

| | Per allele Effect OR | | Genotype counts, | |
|---|---|---|---|---|
| Outcome | (95% confidence interval) | P-value | RR\|RA\|AA genotypes | AAF |
| Parenchymal liver | 0.91 (0.88, 0.93) | 1.60E−13 | Cases: 1,435\|6,619\|7,660 | 0.71 |
| disease | | | Controls: 32,620\|166,799\|219,527 | |
| Non-alcoholic | 0.90 (0.88, 0.93) | 7.20E−13 | Cases: 1,261\|5,520\|6,429 | 0.71 |
| liver disease | | | Controls: 30,928\|158,266\|208,098 | |
| Liver disease | 0.93 (0.91, 0.95) | 2.20E−12 | Cases: 2,016\|9,682\|11,366 | 0.72 |
| (any) | | | Controls: 32,620\|166,799\|219,527 | |
| Liver fibrosis or | 0.85 (0.81, 0.90) | 7.80E−10 | Cases: 341\|1,583\|1,673 | 0.71 |
| cirrhosis | | | Controls: 30,928\|158,266\|208,098 | |
| Non-alcoholic | 0.89 (0.86, 0.93) | 1.60E−09 | Cases: 710\|3,084\|3,575 | 0.72 |
| fatty liver disease | | | Controls: 30,615\|156,760\|206,236 | |
| and steatosis | | | | |
| hepatitis | | | | |
| Liver cirrhosis | 0.86 (0.81, 0.90) | 4.20E−09 | Cases: 318\|1,519\|1,592 | 0.71 |
| | | | Controls: 30,928\|158,266\|208,098 | |
| Non-alcoholic | 0.85 (0.81, 0.90) | 6.80E−09 | Cases: 307\|1,365\|1,452 | 0.71 |
| liver fibrosis or | | | Controls: 30,927\|158,265\|208,095 | |
| cirrhosis | | | | |
| Non-alcoholic | 0.85 (0.81, 0.90) | 2.40E−08 | Cases: 284\|1,298\|1,365 | 0.71 |
| liver cirrhosis | | | Controls: 30,905\|158,181\|207,993 | |
| Alcoholic liver | 0.87 (0.81, 0.93) | 6.70E−05 | Cases: 164\|846\|946 | 0.72 |
| disease | | | Controls: 30,016\|154,665\|204,484 | |
| Alcoholic liver | 0.84 (0.77, 0.91) | 7.90E−05 | Cases: 104\|516\|546 | 0.72 |
| cirrhosis | | | Controls: 30,016\|154,665\|204,484 | |
| Viral hepatitis | 0.94 (0.90, 0.99) | 0.010 | Cases: 396\|1,912\|2,147 | 0.71 |
| | | | Controls: 31,787\|162,679\|213,578 | |

Note:
RR, number of individuals carrying no alternative variant allele (homozygous non-carriers); RA, number of individuals carrying a single alternative variant allele (heterozygous carriers); AA, number of individuals carrying alternative variant alleles in both copies of the gene (homozygous carriers); The alternative allele is the allele causing loss of function or change in amino acid following human genome sequence reference build 38 and HGVS protein sequence nomenclature; AAF indicates the alternative allele frequency; SD indicates standard deviation units; OR indicates odds ratio.

The associations between the burden of loss-of-function variants and PDFF or ALT was driven by multiple missense and loss-of-function variants in GPAM. Table 8 contains all individual variants that were used in the association.

TABLE 8
Chr:position:ref:alt indicates the position of the genetic variant on chromosome (chr) with
reference (ref) and it's alternative (alt) allele on build 38 of the Genome Reference
Consortium. Protein changes follow the recommendation of the Human Genome Variation Society
and correspond to each to the Ensembl transcript IDs, hgvsp (protein change) is given in
case of a protein coding variant, hgvsc (cDNA change) is given in case of a splice variant

| Genomic coordinates, | | |
|---|---|---|
| chr:position:ref:alt | Transcript IDs | Protein or cDNA change |
| 10:112153594:G:A | ENST00000348367 | hgvsp: p.Arg815* |
| 10:112153667:C:T | ENST00000348367 | hgvsc: c.2371 − 1G > A |
| 10:112153668:T:C | ENST00000348367 | hgvsc: c.2371 − 2A > G |
| 10:112157390:C:T | ENST00000348367, | hgvsc: c.1981 − 1G > A, c.1981 − 1G > A |
| | ENST00000369425 | |
| 10:112158315:C:T | ENST00000348367, | hgvsc: c.1980 + 1G > A, c.1980 + 1G > A |
| | ENST00000369425 | |
| 10:112159909:AC:A | ENST00000348367, | hgvsc: c.1902 + 1delG, c.1902 + 1delG |
| | ENST00000369425 | |
| 10:112159915:G:T | ENST00000348367, | hgvsp: p.Ser633*, p.Ser633* |
| | ENST00000369425 | |
| 10:112159955:CCG:C | ENST00000348367, | hgvsp: p.Ala619fs, p.Ala619fs |
| | ENST00000369425 | |
| 10:112160868:C:A | ENST00000348367, | hgvsp: p.Gly499*, p.Gly499* |
| | ENST00000369425 | |
| 10:112163743:G:A | ENST00000348367, | hgvsp: p.Arg461*, p.Arg461* |
| | ENST00000369425 | |
| 10:112166434:G:A | ENST00000348367, | hgvsp: p.Arg397*, p.Arg397* |
| | ENST00000369425 | |
| 10:112166474:TCTTG:T | ENST00000348367, | hgvsp: p.Ala382fs, p.Ala382fs |
| | ENST00000369425 | |
| 10:112168400:GACAGAGTATCT:G | ENST00000348367, | hgvsp: p.Asp337fs, p.Asp337fs |
| | ENST00000369425 | |
| 10:112168506:G:A | ENST00000348367, | hgvsp: p.Arg305*, p.Arg305* |
| | ENST00000369425 | |
| 10:112168915:G:A | ENST00000348367, | hgvsp: p.Arg278*, p.Arg278* |
| | ENST00000369425 | |
| 10:112175598:A:T | ENST00000348367, | hgvsc: c.413 + 2T > A, c.413 + 2T > A |
| | ENST00000369425 | |
| 10:112177984:CT:C | ENST00000348367, | hgvsp: p.Arg100fs, p.Arg100fs |
| | ENST00000369425 | |
| 10:112177986:TG:T | ENST00000348367, | hgvsp: p.Thr99fs, p.Thr99fs |
| | ENST00000369425 | |
| 10:112181719:GT:G | ENST00000348367, | hgvsp: p.Tyr22fs, p.Tyr22fs |
| | ENST00000369425 | |
| 10:112181729:G:C | ENST00000348367, | hgvsp: p.Ser19*, p.Ser19* |
| | ENST00000369425 | |
| 10:112156032:G:A | ENST00000348367 | hgvsp: p.Gln715* |
| 10:112158343:GA:G | ENST00000348367, | hgvsp: p.Ile651fs, p.Ile651fs |
| | ENST00000369425 | |
| 10:112160010:AC:A | ENST00000348367, | hgvsp: p.Gly601fs, p.Gly601fs |
| | ENST00000369425 | |
| 10:112161731:C:CT | ENST00000348367, | hgvsp: p.Ser477fs, p.Ser477fs |
| | ENST00000369425 | |
| 10:112163779:TG:T | ENST00000348367, | hgvsp: p.Ile449fs, p.Ile449fs |
| | ENST00000369425 | |
| 10:112163790:CT:C | ENST00000348367, | hgvsp: p.Arg445fs, p.Arg445fs |
| | ENST00000369425 | |
| 10:112166419:G:A | ENST00000348367, | hgvsp: p.Gln402*, p.Gln402* |
| | ENST00000369425 | |
| 10:112166455:G:A | ENST00000348367, | hgvsp: p.Arg390*, p.Arg390* |
| | ENST00000369425 | |
| 10:112166517:T:A | ENST00000348367, | hgvsc: c.1108 − 2A > T, c.1108 − 2A > T |
| | ENST00000369425 | |
| 10:112168309:TA:T | ENST00000348367, | hgvsc: c.1107 + 2delT, c.1107 + 2delT |
| | ENST00000369425 | |
| 10:112168351:A:T | ENST00000348367, | hgvsp: p.Tyr356*, p.Tyr356* |
| | ENST00000369425 | |
| 10:112168503:G:A | ENST00000348367, | hgvsp: p.Gln306*, p.Gln306* |
| | ENST00000369425 | |
| 10:112168953:C:T | ENST00000348367, | hgvsc: c.795 − 1G > A, c.795 − 1G > A |
| | ENST00000369425 | |
| 10:112173052:AC:A | ENST00000348367, | hgvsp: p.Val192fs, p.Val192fs |
| | ENST00000369425 | |
| 10:112173717:AC:A | ENST00000348367, | hgvsp: p.Val181fs, p.Val181fs |
| | ENST00000369425 | |
| 10:112173846:C:T | ENST00000348367, | hgvsc: c.414 − 1G > A, c.414 − 1G > A |
| | ENST00000369425 | |
| 10:112173847:T:C | ENST00000348367, | hgvsc: c.414 − 2A > G, c.414 − 2A > G |
| | ENST00000369425 | |
| 10:112175661:G:A | ENST00000348367, | hgvsp: p.Arg118*, p.Arg118* |
| | ENST00000369425 | |
| 10:112177985:TTG:T | ENST00000348367, | hgvsp: p.Thr99fs, p.Thr99fs |
| | ENST00000369425 | |
| 10:112181683:C:T | ENST00000348367, | hgvsp: p.Trp34*, p.Trp34* |
| | ENST00000369425 | |
| 10:112181784:T:C | ENST00000348367, | hgvsp: p.Met1?, p.Met1? |
| | ENST00000369425 | |
| 10:112153559:CACAAA:C | ENST00000348367 | hgvsp: p.Phe825fs |
| 10:112153575:TA:T | ENST00000348367 | hgvsp: p.Tyr821fs |
| 10:112153591:G:A | ENST00000348367 | hgvsp: p.Gln816* |
| 10:112153603:G:A | ENST00000348367 | hgvsp: p.Gln812* |
| 10:112153631:AAC:A | ENST00000348367 | hgvsp: p.Val802fs |
| 10:112154628:C:T | ENST00000348367 | hgvsc: c.2370 + 1G > A |
| 10:112154636:TC:T | ENST00000348367 | hgvsp: p.Asp788fs |
| 10:112154678:GC:G | ENST00000348367 | hgvsp: p.Ala774fs |
| 10:112155862:A:T | ENST00000348367 | hgvsc: c.2311 + 2T > A |
| 10:112155899:AG:A | ENST00000348367 | hgvsp: p.Leu759fs |
| 10:112156055:T:C | ENST00000348367 | hgvsc: c.2122 − 2A > G |
| 10:112157247:A:T | ENST00000348367 | hgvsc: c.2121 + 2T > A |
| 10:112157266:G:A | ENST00000348367, | hgvsp: p.Arg702*, p.Arg702* |
| | ENST00000369425 | |
| 10:112157273:C:CT | ENST00000348367, | hgvsp: p.Glu700fs, p.Glu700fs |
| | ENST00000369425 | |
| 10:112157338:T:A | ENST00000348367, | hgvsp: p.Lys678*, p.Lys678* |
| | ENST00000369425 | |
| 10:112157371:TATCTTCCTGG:T | ENST00000348367, | hgvsp: p.Asp663fs, p.Asp663fs |
| | ENST00000369425 | |
| 10:112158319:TG:T | ENST00000348367, | hgvsp: p.Ala659fs, p.Ala659fs |
| | ENST00000369425 | |
| 10:112158326:GT:G | ENST00000348367, | hgvsp: p.Thr657fs, p.Thr657fs |
| | ENST00000369425 | |
| 10:112158367:G:T | ENST00000348367, | hgvsp: p.Cys643*, p.Cys643* |
| | ENST00000369425 | |
| 10:112158375:G:A | ENST00000348367, | hgvsp: p.Gln641*, p.Gln641* |
| | ENST00000369425 | |
| 10:112159915:G:C | ENST00000348367, | hgvsp: p.Ser633*, p.Ser633* |
| | ENST00000369425 | |
| 10:112159941:G:T | ENST00000348367, | hgvsp: p.Tyr624*, p.Tyr624* |
| | ENST00000369425 | |
| 10:112160010:A:AC | ENST00000348367, | hgvsp: p.Pro602fs, p.Pro602fs |
| | ENST00000369425 | |
| 10:112160603:C:A | ENST00000348367, | hgvsc: c.1759 + 1G > T, c.1759 + 1G > T |
| | ENST00000369425 | |
| 10:112160620:GAT:G | ENST00000348367, | hgvsp: p.Ile581fs, p.Ile581fs |
| | ENST00000369425 | |
| 10:112160685:TG:T | ENST00000348367, | hgvsp: p.Ser560fs, p.Ser560fs |
| | ENST00000369425 | |
| 10:112160685:T:TG | ENST00000348367, | hgvsp: p.Ser560fs, p.Ser560fs |
| | ENST00000369425 | |
| 10:112160694:T:TA | ENST00000348367, | hgvsp: p.Ile557fs, p.Ile557fs |
| | ENST00000369425 | |
| 10:112160786:G:T | ENST00000348367, | hgvsp: p.Ser526*, p.Ser526* |
| | ENST00000369425 | |
| 10:112160854:GGA:G | ENST00000348367, | hgvsp: p.Ser503fs, p.Ser503fs |
| | ENST00000369425 | |
| 10:112161666:C:T | ENST00000348367, | hgvsc: c.1494 + 1G > A, c.1494 + 1G > A |
| | ENST00000369425 | |
| 10:112161739:T:A | ENST00000348367, | hgvsc: c.1424 − 2A > T, c.1424 − 2A > T |
| | ENST00000369425 | |
| 10:112163718:GC:G | ENST00000348367, | hgvsp: p.Ala469fs, p.Ala469fs |
| | ENST00000369425 | |
| 10:112163744:TA:T | ENST00000348367, | hgvsp: p.Leu460fs, p.Leu460fs |
| | ENST00000369425 | |
| 10:112163748:G:GT | ENST00000348367, | hgvsp: p.Ser459fs, p.Ser459fs |
| | ENST00000369425 | |
| 10:112163796:TC:T | ENST00000348367, | hgvsp: p.Glu443fs, p.Glu443fs |
| | ENST00000369425 | |
| 10:112163812:TG:T | ENST00000348367, | hgvsp: p.Ser438fs, p.Ser438fs |
| | ENST00000369425 | |
| 10:112164527:TG:T | ENST00000348367, | hgvsp: p.Ser435fs, p.Ser435fs |
| | ENST00000369425 | |
| 10:112164527:TGAAG:T | ENST00000348367, | hgvsp: p.Pro434fs, p.Pro434fs |
| | ENST00000369425 | |
| 10:112164528:GA:G | ENST00000348367, | hgvsp: p.Ser435fs, p.Ser435fs |
| | ENST00000369425 | |
| 10:112164528:G:T | ENST00000348367, | hgvsp: p.Ser435*, p.Ser435* |
| | ENST00000369425 | |
| 10:112164538:TA:T | ENST00000348367, | hgvsp: p.Ile432fs, p.Ile432fs |
| | ENST00000369425 | |
| 10:112164541:CT:C | ENST00000348367, | hgvsp: p.Ala431fs, p.Ala431fs |
| | ENST00000369425 | |
| 10:112164542:TG:T | ENST00000348367, | hgvsp: p.Pro430fs, p.Pro430fs |
| | ENST00000369425 | |
| 10:112164558:TC:T | ENST00000348367, | hgvsp: p.Glu425fs, p.Glu425fs |
| | ENST00000369425 | |
| 10:112164561:AG:A | ENST00000348367, | hgvsp: p.Leu424fs, p.Leu424fs |
| | ENST00000369425 | |
| 10:112164597:CT:C | ENST00000348367, | hgvsp: p.Ser412fs, p.Ser412fs |
| | ENST00000369425 | |
| 10:112164601:CT:C | ENST00000348367, | hgvsp: p.Glu411fs, p.Glu411fs |
| | ENST00000369425 | |
| 10:112164607:AT:A | ENST00000348367, | hgvsp: p.Glu408fs, p.Glu408fs |
| | ENST00000369425 | |
| 10:112164611:C:T | ENST00000348367, | hgvsc: c.1222 − 1G > A, c.1222 − 1G > A |
| | ENST00000369425 | |
| 10:112164612:T:C | ENST00000348367, | hgvsc: c.1222 − 2A > G, c.1222 − 2A > G |
| | ENST00000369425 | |
| 10:112166409:GA:G | ENST00000348367, | hgvsp: p.Ser405fs, p.Ser405fs |
| | ENST00000369425 | |
| 10:112166443:CAT:C | ENST00000348367, | hgvsp: p.Tyr393fs, p.Tyr393fs |
| | ENST00000369425 | |
| 10:112166497:C:CA | ENST00000348367, | hgvsp: p.Glu376fs, p.Glu376fs |
| | ENST00000369425 | |
| 10:112166516:CT:C | ENST00000348367, | hgvsc: c.1108 − 2delA, c.1108 − 2delA |
| | ENST00000369425 | |
| 10:112168327:G:T | ENST00000348367, | hgvsp: p.Tyr364*, p.Tyr364* |
| | ENST00000369425 | |
| 10:112168335:CT:C | ENST00000348367, | hgvsp: p.Gly362fs, p.Gly362fs |
| | ENST00000369425 | |
| 10:112168411:TAC:T | ENST00000348367, | hgvsp: p.Val336fs, p.Val336fs |
| | ENST00000369425 | |
| 10:112168857:TG:T | ENST00000348367, | hgvsp: p.His297fs, p.His297fs |
| | ENST00000369425 | |
| 10:112168872:TA:T | ENST00000348367, | hgvsp: p.Tyr292fs, p.Tyr292fs |
| | ENST00000369425 | |
| 10:112168918:G:A | ENST00000348367, | hgvsp: p.Arg277*, p.Arg277* |
| | ENST00000369425 | |
| 10:112168937:CT:C | ENST00000348367, | hgvsp: p.Lys270fs, p.Lys270fs |
| | ENST00000369425 | |
| 10:112168939:T:A | ENST00000348367, | hgvsp: p.Lys270*, p.Lys270* |
| | ENST00000369425 | |
| 10:112172190:TG:T | ENST00000348367, | hgvsp: p.Pro262fs, p.Pro262fs |
| | ENST00000369425 | |
| 10:112172968:G:T | ENST00000348367, | hgvsc: c.657 + 2C > A, c.657 + 2C > A |
| | ENST00000369425 | |
| 10:112172968:G:C | ENST00000348367, | hgvsc: c.657 + 2C > G, c.657 + 2C > G |
| | ENST00000369425 | |
| 10:112172972:CA:C | ENST00000348367, | hgvsp: p.Glu219fs, p.Glu219fs |
| | ENST00000369425 | |
| 10:112172976:TG:T | ENST00000348367, | hgvsp: p.Ala217fs, p.Ala217fs |
| | ENST00000369425 | |
| 10:112173035:TG:T | ENST00000348367, | hgvsp: p.Phe197fs, p.Phe197fs |
| | ENST00000369425 | |
| 10:112173050:GCACC:G | ENST00000348367, | hgvsp: p.Trp191fs, p.Trp191fs |
| | ENST00000369425 | |
| 10:112173056:AC:A | ENST00000348367, | hgvsp: p.Trp191fs, p.Trp191fs |
| | ENST00000369425 | |
| 10:112173058:CCAGT:C | ENST00000348367, | hgvsp: p.Thr189fs, p.Thr189fs |
| | ENST00000369425 | |
| 10:112173067:C:A | ENST00000348367, | hgvsc: c.561 − 1G > T, c.561 − 1G > T |
| | ENST00000369425 | |
| 10:112173733:C:A | ENST00000348367, | hgvsp: p.Glu176*, p.Glu176* |
| | ENST00000369425 | |
| 10:112175598:A:G | ENST00000348367, | hgvsc: c.413 + 2T > C, c.413 + 2T > C |
| | ENST00000369425 | |
| 10:112175599:C:T | ENST00000348367, | hgvsc: c.413 + 1G > A, c.413 + 1G > A |
| | ENST00000369425 | |
| 10:112175680:G:C | ENST00000348367, | hgvsp: p.Tyr111*, p.Tyr111* |
| | ENST00000369425 | |
| 10:112177983:C:A | ENST00000348367, | hgvsc: c.299 + 1G > T, c.299 + 1G > T |
| | ENST00000369425 | |
| 10:112178039:TG:T | ENST00000348367, | hgvsp: p.Ser82fs, p.Ser82fs |
| | ENST00000369425 | |
| 10:112178058:C:G | ENST00000348367, | hgvsc: c.226 − 1G > C, c.226 − 1G > C |
| | ENST00000369425 | |
| 10:112178058:C:A | ENST00000348367, | hgvsc: c.226 − 1G > T, c.226 − 1G > T |
| | ENST00000369425 | |
| 10:112178059:T:C | ENST00000348367, | hgvsc: c.226 − 2A > G, c.226 − 2A > G |
| | ENST00000369425 | |
| 10:112180473:C:T | ENST00000348367, | hgvsp: p.Trp75*, p.Trp75* |
| | ENST00000369425 | |
| 10:112180580:T:A | ENST00000348367, | hgvsp: p.Arg40*, p.Arg40* |
| | ENST00000369425 | |
| 10:112180597:T:C | ENST00000348367, | hgvsc: c.103 − 2A > G, c.103 − 2A > G |
| | ENST00000369425 | |
| 10:112181681:AC:A | ENST00000348367, | hgvsc: c.102 + 1delG, c.102 + 1delG |
| | ENST00000369425 | |
| 10:112181691:CACTT:C | ENST00000348367, | hgvsp: p.Ser31fs, p.Ser31fs |
| | ENST00000369425 | |
| 10:112181694:TTG:T | ENST00000348367, | hgvsp: p.Thr30fs, p.Thr30fs |
| | ENST00000369425 | |
| 10:112181709:G:A | ENST00000348367, | hgvsp: p.Arg26*, p.Arg26* |
| | ENST00000369425 | |
| 10:112181710:AC:A | ENST00000348367, | hgvsp: p.Gly25fs, p.Gly25fs |
| | ENST00000369425 | |
| 10:112181729:G:T | ENST00000348367, | hgvsp: p.Ser19*, p.Ser19* |
| | ENST00000369425 | |
| 10:112181734:TG:T | ENST00000348367, | hgvsp: p.Pro17fs, p.Pro17fs |
| | ENST00000369425 | |
| 10:112181740:A:C | ENST00000348367, | hgvsp: p.Tyr15*, p.Tyr15* |
| | ENST00000369425 | |
| 10:112181757:TA:T | ENST00000348367, | hgvsp: p.Thr10fs, p.Thr10fs |
| | ENST00000369425 | |
| 10:112181780:TCC:T | ENST00000348367, | hgvsp: p.Met1fs, p.Met1fs |
| | ENST00000369425 | |
| 10:112181783:A:C | ENST00000348367, | hgvsp: p.Met1?, p.Met1? |
| | ENST00000369425 | |
| 10:112181815:T:C | ENST00000348367, | hgvsc: c.−29 − 2A > G, c.−29 − 2A > G |
| | ENST00000369425 | |
| 10:112153552:A:G | ENST00000348367 | hgvsp: p.Ter829Glnext*? |
| 10:112157248:C:A | ENST00000348367 | hgvsc: c.2121 + 1G > T |
| 10:112157343:C:T | ENST00000348367, | hgvsp: p.Trp676*, p.Trp676* |
| | ENST00000369425 | |
| 10:112160805:CACGAGCCAGG:C | ENST00000348367, | hgvsp: p.Leu517fs, p.Leu517fs |
| | ENST00000369425 | |
| 10:112168525:C:T | ENST00000348367, | hgvsc: c.895 − 1G > A, c.895 − 1G > A |
| | ENST00000369425 | |
| 10:112172320:T:C | ENST00000348367, | hgvsc: c.658 − 2A > G, c.658 − 2A > G |
| | ENST00000369425 | |
| 10:112173037:AAC:A | ENST00000348367, | hgvsp: p.Phe197fs, p.Phe197fs |
| | ENST00000369425 | |
| 10:112164564:G:GA | ENST00000348367, | hgvsp: p.Ser423fs, p.Ser423fs |
| | ENST00000369425 | |
| 10:112166441:AC:A | ENST00000348367, | hgvsp: p.Gly394fs, p.Gly394fs |
| | ENST00000369425 | |
| 10:112168850:T:TA | ENST00000348367, | hgvsc: c.894 + 2_894 + 3insT, c.894 + |
| | ENST00000369425 | 2_894 + 3insT |
| 10:112172202:AT:A | ENST00000348367, | hgvsp: p.Asn258fs, p.Asn258fs |
| | ENST00000369425 | |
| 10:112173754:TC:T | ENST00000348367, | hgvsp: p.Ala170fs, p.Ala170fs |
| | ENST00000369425 | |
| 10:112173790:G:A | ENST00000348367, | hgvsp: p.Gln157*, p.Gln157* |
| | ENST00000369425 | |
| 10:112181682:C:T | ENST00000348367, | hgvsc: c.102 + 1G > A, c.102 + 1G > A |
| | ENST00000369425 | |
| 10:112181814:C:A | ENST00000348367, | hgvsc: c.−29 − 1G > T, c.−29 − 1G > T |
| | ENST00000369425 | |
| 10:112155956:TG:T | ENST00000348367 | hgvsp: p.His740fs |
| 10:112158327:TA:T | ENST00000348367, | hgvsp: p.Thr657fs, p.Thr657fs |
| | ENST00000369425 | |
| 10:112158363:C:A | ENST00000348367, | hgvsp: p.Glu645*, p.Glu645* |
| | ENST00000369425 | |
| 10:112160869:C:T | ENST00000348367, | hgvsc: c.1495 − 1G > A, c.1495 − 1G > A |
| | ENST00000369425 | |
| 10:112163818:T:C | ENST00000348367, | hgvsc: c.1308 − 2A > G, c.1308 − 2A > G |
| | ENST00000369425 | |
| 10:112166400:A:T | ENST00000348367, | hgvsc: c.1221 + 2T > A, c.1221 + 2T > A |
| | ENST00000369425 | |
| 10:112166401:C:T | ENST00000348367, | hgvsc: c.1221 + 1G > A, c.1221 + 1G > A |
| | ENST00000369425 | |
| 10:112168951:TA:T | ENST00000348367, | hgvsp: p.Ser265fs, p.Ser265fs |
| | ENST00000369425 | |
| 10:112168954:T:C | ENST00000348367, | hgvsc: c.795 − 2A > G, c.795 − 2A > G |
| | ENST00000369425 | |
| 10:112178004:A:T | ENST00000348367, | hgvsp: p.Tyr93*, p.Tyr93* |
| | ENST00000369425 | |
| 10:112178005:TA:T | ENST00000348367, | hgvsp: p.Tyr93fs, p.Tyr93fs |
| | ENST00000369425 | |

### Participating Cohorts:

Genetic association studies were performed in the United Kingdom Biobank (UKB) cohort (Bycroft et al., 2018, doi:10.1038/s41586-018-0579-z; Van Hout et al., 2020, doi:10.1038/s41586-020-2853-0), the MyCode Community Health Initiative cohort from the Geisinger Health System (GHS) (Carey et al., 2016, doi:10.1038/gim.2015.187) and Mount Sinai's BioMe Personalized Medicine Cohort (SINAI) (Gottesman et al., 2013, doi:10.1038/gim.2013.72).

The UKB is a population-based cohort study of people aged between 40 and 69 years recruited through 22 testing centers in the UK between 2006-2010. A total of 430,998 European ancestry participants from UKB with available whole-exome sequencing and phenotype data were included. The GHS MyCode study is a health system-based cohort of patients from Central and Eastern Pennsylvania (USA) recruited in 2007-2019. A total of 136,239 European ancestry participants from GHS with available whole-exome sequencing and phenotype data were included. Mount Sinai's BioMe Personalized Medicine Cohort (SINAI) is an electronic health record-linked clinical care cohort of 30,619 individuals. These individuals are from diverse ancestries and characterized by a broad spectrum of biomedical traits.

### Liver MRI Phenotypes:

A subset of 40,058 individuals participating in UKB underwent imaging sessions of liver magnetic resonance imaging (MRI) (Littlejohns et al., 2020, doi:10.1038/s41467-020-15948-9). The majority of UK Biobank participants selected for liver MRI underwent two acquisitions, one for estimating fat content and the other a quantitative T1 mapping sequence. For the former, approximately 10,000 subjects were imaged under a Dixon gradient echo protocol; in 2016, the acquisition protocol for measurement of fat fraction was updated to the IDEAL sequence (Iterative Decomposition of water and fat with Echo Asymmetry and Least-squares estimation). Data from this acquisition are provided as a series of complex-valued 2D images per subject. The in-plane pixel size is 2.5×2.5 mm; slice thickness is 6 mm. The latter protocol, “ShMOLLI” (Shortened Modified Look-Locker Inversion recovery), has been consistent throughout the study. Data for this acquisition are provided as one real-valued 2D pre-computed T1 map per subject. The in-plane pixel size is 1.15×1.15 mm; slice thickness is 8 mm. Both MRI datasets were acquired at the same 2D cross-section per subject, intended to be through the porta hepatis. All images were acquired on a Siemens MAGNETOM Aera 1.5T clinical MRI scanner.

Measurements of PDFF, ECF, T1 and corrected T1 were obtained by applying pre-defined mathematical models after segmenting the liver on liver MRI images (Hernando et al., 2012, doi:10.1002/mrm.23044; Wood et al., 2005, doi:10.1182/blood-2004-10-3982; Tunnicliffe et al., 2017, doi:10.1002/jmri.25392). Three distinct phenotypes were derived from two abdominal MRIs acquisition, one for estimating fat content and the other a quantitative T1 mapping sequence: proton density fat fraction (PDFF), and extracellular fluid fraction (ECF, a proxy for liver fibrosis and inflammation). PDFF was estimated as the fraction of fat signal relative to total fat plus water signal. ECF was estimated by interpolation from their published Table containing grid points of a non-linear numerical model describing ECF as a function of T1 (from ShMOLLI MRI) and hepatic iron content (from IDEAL MRI), correcting for field strength.

Pixels belonging to the liver were segmented using a thresholding approach, Li thresholding for PDFF maps to identify liver tissue, and Otsu thresholding for T1 maps to exclude larger vessels. To obtain a summary measure of each trait per subject, all pixels within the liver were averaged for each parametric map.

All traits were deconfounded by residualizing the traits with the following covariates: sex, age, age-squared, top 20 principal components for ancestry, age*sex, imaging center, imaging protocol. Additional covariates (referred to as ‘extra’ here), were BMI, BMI², 7 binary alcohol variables (daily, 1-2 times per week, 3-4 times per week, 1-3 times per month, special occasions, previous, current), 2 binary weight gain variables (weight gain in last year, weight loss in last year) and 5 binary disease variables (diabetes, heart attack, angina, stroke, high blood pressure).

### Phenotype Definitions:

Clinical laboratory measurements for ALT, LDL-C, HDL-C, triglycerides, BMI, waist, hip, glucose and Hb1Ac were extracted from electronic health records (EHRs) of participants from GHS or measured from blood at UKB recruitment centers. For GHS, median values were calculated for all participants with two or more measurements. In UKB, ALT, LDL-C, HDL-C, triglyceride and glucose were measured by IFCC (International Federation of Clinical Chemistry) analysis on a Beckman Coulter AU5800 at the baseline visit of the study and averaged in case of multiple measurements. Hb1Ac was measured by HPLC using a Bio-Rad VARIANT II Turbo. Prior to genetic association analysis, continues phenotype values were transformed by the inverse standard normal function, applied within each ancestry group and separately in men and women.

In GHS, UKB and SINAI, disease outcomes were defined according to the International Classification of Diseases, Ninth and Tenth Revision (ICD-9 and ICD-t) and Read codes stored in EHRs, and self-reports were used when available; all of which and combined into single variables to classify individuals into cases or controls. Individuals with type 2 diabetes were identified using a previously described algorithm (Eastwood et al., 2016, doi:10.1371/journal.pone.0162388). Individuals with coronary artery disease or liver diseases were identified as described in Table 9, combining EHR records, self-reports and ALT measurements.

TABLE 9
Individuals with or without disease were identified in GHS, UKB and SINAI using EHR records
and self-reports. OPCS4 codes (operation procedures), f.20002 (self-reported disease)
and f.20004 (self-reported operation procedures) variables were specific to UKB. In
each cohort, EHR records with ICD-9 or read codes were translated to ICD-10 codes

| | Case definition | Control exclusion |
|---|---|---|
| Parenchymal | ICD10: | ICD10: |
| liver disease | K70, K71, K72, K73, K74, K753, K753, | K70, K71, K72, K73, K74, K75, K76, K77, I81, |
| | K752, K754, K758, K759, K760, | I85, I982, I983, I864, T864, Z944, C220 |
| | K767, K7681 | OPCS4: G10, G144, J01 |
| | OPCS4: G10, G144, J01 | f.20002: 1604, 1158, 1141 |
| | f.20002: 1604, 1158, 1141 | ALT: >33 IU/L for men and >24 IU/L for women |
| Non- | ICD10: K746, K758, K760 | See Parenchymal liver disease |
| alcoholic | | |
| liver disease | | |
| Non- | ICD10: | See Parenchymal liver disease |
| alcoholic | K721, K740, K741, K742, K746, | |
| liver disease | K758, K760 | |
| (broad) | | |
| Liver disease | ICD10: K70, K71, K72, K73, K74, K75, K76, | See Parenchymal liver disease |
| (any) | K77, I81, I85, I982, I983, I864, T864, Z944, | |
| | C220 | |
| | OPCS4: G10, G144, J01 | |
| | f.20002: 1604, 1158, 1141 | |
| Liver fibrosis | ICD10: | See Parenchymal liver disease |
| or cirrhosis | K703, K704, K717, K721, K746, K702, | |
| (agnostic) | K740, K741, K742 | |
| Non- | ICD10: K760, K7581 | See Parenchymal liver disease |
| alcoholic | | |
| fatty liver | | |
| disease and | | |
| steatosis | | |
| hepatitis | | |
| Liver | ICD10: K703, K704, K717, K721, K746 | See Parenchymal liver disease |
| cirrhosis | | |
| Non- | ICD10: K721, K740, K741, K742, K746 | See Parenchymal liver disease |
| alcoholic | | |
| Liver fibrosis | | |
| or cirrhosis | | |
| Non- | ICD10: K746 | See Parenchymal liver disease |
| alcoholic | | |
| liver cirrhosis | | |
| Alcoholic | ICD10: K70 | See Parenchymal liver disease |
| liver disease | | |
| Alcoholic | ICD10: K703, K704 | See Parenchymal liver disease |
| liver cirrhosis | | |
| Viral | ICD10: K746, K758, K760 | See Parenchymal liver disease |
| hepatitis | | |
| Coronary | ICD10: Z955, I248, I249, I241, I251, I255, | Family history of heart disease based on |
| artery | I258, I259, I21, I22, I23, I252, I256, Z951 | ICD10: Z824 |
| disease | OPSC4: K471, K49, K502, K75, K40, K41, | self-reports in UK Biobank (fields 20107, |
| | K44, K45, K46 | 20110 and 20111) |
| | f.20002: 1075 | |
| | f.20004: 1070, 1095, 1523 | |
| | EHR: CABG or stenosis from Cath-lab | |
| | records. | |

### Genotype Data:

High coverage whole exome sequencing was performed as previously described (Science, 2016, 354:aaf6814; and Nature, 2020; 586, 749-756) and as summarized below. NimbleGen probes (VCRome; for part of the GHS cohort) or a modified version of the xGen design available from Integrated DNA Technologies (IDT; for the rest of GHS and other cohorts) were used for target sequence capture of the exome. A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina v4 HiSeq 2500 (for part of the GHS cohort) or NovaSeq (for the rest of GHS and other cohorts) instruments. Sequencing had a coverage depth (i.e., number of sequence-reads covering each nucleotide in the target areas of the genome) sufficient to provide greater than 20× coverage over 85% of targeted bases in 96% of VCRome samples and 20× coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (DOI: 10.1101/343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Adzhubei et al., Nat. Methods, 2010, 7, 248-9) and Polyphen2_HVAR (Adzhubei et al., Nat. Methods, 2010, 7, 248-9), LRT (Chun et al., Genome Res., 2009, 19, 1553-61) and MutationTaster (Schwarz et al., Nat. Methods, 2010, 7, 575-6). For each gene, the alternative allele frequency (AAF) and functional annotation of each variant determined inclusion into these 7 gene burden exposures: 1) pLOF variants with AAF<1%; 2) pLOF or missense variants predicted deleterious by 5/5 algorithms with AAF<1%; 3) pLOF or missense variants predicted deleterious by 5/5 algorithms with AAF<0.1%; 4) pLOF or missense variants predicted deleterious by at least 1/5 algorithms with AAF<1%; 5) pLOF or missense variants predicted deleterious by at least 1/5 algorithms with AAF<0.1%; 6) pLOF or any missense with AAF<1%; 7) pLOF or any missense variants with AAF<0.1%.

### Association Analysis of Gene Burden of Rare Loss of Function Variation:

Association between the burden of rare predicted loss-of-function or missense variants in a given gene and phenotype was examined by fitting a linear (for quantitative traits) or firth bias-corrected logistic (for binary traits) regression model adjusted for a polygenic score that approximates a genomic kinship matrix using REGENIE v1.0 (doi: world wide web at “doi.org/10.1101/2020.06.19.162354”). Analyses were stratified by ancestry and adjusted for age, age², sex, age-by-sex and age²-by-sex interaction terms, experimental batch-related covariates, 10 common variant-derived principal components, and 20 rare variant-derived principal components. Results across cohorts for each variant-phenotype association were combined using fixed effects inverse variance weighted meta-analysis. In gene burden tests, all individuals are labeled as heterozygotes if they carry one or more qualifying rare variant (as described above based on frequency and functional annotation) and as homozygotes if they carry any qualifying variant in the homozygous state. This “composite genotype” is then used to test for association.

### GWAS of Common Variants:

Associated common variants were identified by performing a genome-wide association study including over 12 million common-to-low-frequency genetic variants imputed using the Haplotype Reference Consortium panel. In the GHS study, imputation was performed separately in samples genotyped with the Illumina Human Omni Express Exome array (OMNI set) and the Global Screening array (GSA set). Dosage data from imputed variants were then merged across the two GHS sets, to obtain a combined dataset for association analysis. Genome-wide association analyses were performed in GHS and UKB separately by fitting whole genome regression models using REGENIE (Mbatchou et al., 2020, doi:10.1101/2020.06.19.162354). As described above for burden tests, within each cohort analyses were stratified by ancestry and adjusted for age, age², sex, age-by-sex and age²-by-sex interaction terms, experimental batch-related covariates, and 10 common variant-derived principal components. Results from the UKB and GHS analyses were then combined by inverse variance-weighted meta-analysis to obtain a genome-wide meta-analysis in the European subset of the discovery cohorts.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having liver disease or at risk of having liver disease, having fatty liver disease or at risk of having fatty liver disease, having hepatocellular carcinoma or at risk of having hepatocellular carcinoma, having liver cirrhosis or at risk of having liver cirrhosis, having liver fibrosis or at risk of having liver fibrosis, having simple steatosis, steatohepatitis, or non-alcoholic steatohepatitis (NASH) or at risk of having simple steatosis, steatohepatitis, or NASH, the method comprising administering a glycerol-3-phosphate acyltransferase (GPAM) inhibitor to the subject.

2-36. (canceled)

37. A method of treating a subject with a therapeutic agent that treats or inhibits liver disease and/or a glycerol-3-phosphate acyltransferase (GPAM) inhibitor, wherein the subject is suffering from liver disease, fatty liver disease, hepatocellular carcinoma, liver cirrhosis, liver fibrosis, simple steatosis, steatohepatitis, or non-alcoholic steatohepatitis (NASH), the method comprising the steps of:
determining whether the subject has a GPAM predicted loss-of-function variant nucleic acid molecule encoding a human GPAM polypeptide by:
obtaining or having obtained a biological sample from the subject; and
performing or having performed a sequence analysis on the biological sample to determine if the subject has a genotype comprising the GPAM predicted loss-of-function variant nucleic acid molecule; and
when the subject is GPAM reference, then administering or continuing to administer to the subject the therapeutic agent that treats or inhibits liver disease in a standard dosage amount, and administering to the subject a GPAM inhibitor; and
when the subject is heterozygous for a GPAM predicted loss-of-function variant nucleic acid molecule, then administering or continuing to administer to the subject the therapeutic agent that treats or inhibits liver disease in an amount that is the same as or lower than a standard dosage amount, and administering to the subject a GPAM inhibitor;
wherein the presence of a genotype having the GPAM predicted loss-of-function variant nucleic acid molecule encoding the human GPAM polypeptide indicates the subject has a reduced risk of developing liver disease.

38. The method according to claim 37, wherein the subject is GPAM reference, and the subject is administered or continued to be administered the therapeutic agent that treats or inhibits liver disease in a standard dosage amount, and is administered a GPAM inhibitor.

39. The method according to claim 37, wherein the subject is heterozygous for a GPAM predicted loss-of-function variant nucleic acid molecule, and the subject is administered or continued to be administered the therapeutic agent that treats or inhibits liver disease in an amount that is the same as or lower than a standard dosage amount, and is administered a GPAM inhibitor.

40. The method according to claim 37, wherein the GPAM predicted loss-of-function variant nucleic acid molecule is a nucleic acid molecule encoding GPAM Ile43Val.

41. The method according to claim 37, wherein the GPAM predicted loss-of-function variant nucleic acid molecule is:
a genomic nucleic acid molecule comprising SEQ ID NO:2;
an mRNA molecule comprising comprising SEQ ID NO:9, SEQ ID NO:10, SEQ ID NO:11, SEQ ID NO:12, SEQ ID NO:13, or SEQ ID NO:14; and/or
a cDNA molecule produced from an mRNA molecule in the biological sample, wherein the cDNA molecule comprises SEQ ID NO:21, SEQ ID NO:22, SEQ ID NO:23, SEQ ID NO:24, SEQ ID NO:25, or SEQ ID NO:26.

42. The method according to claim 41, wherein the sequence analysis comprises sequencing at least a portion of the nucleotide sequence of the GPAM genomic nucleic acid molecule in the biological sample, wherein the sequenced portion comprises a position corresponding to position 3,195 according to SEQ ID NO:2, or the complement thereof;
wherein when the sequenced portion of the GPAM genomic nucleic acid molecule in the biological sample comprises a guanine at a position corresponding to position 3,195 according to SEQ ID NO:2, then the GPAM genomic nucleic acid molecule in the biological sample is a GPAM predicted loss-of-function variant genomic nucleic acid molecule.

43. The method according to claim 41, wherein the sequence analysis comprises sequencing at least a portion of the nucleotide sequence of the GPAM mRNA molecule in the biological sample, wherein the sequenced portion comprises a position corresponding to: position 327 according to SEQ ID NO:9, or the complement thereof; position 291 according to SEQ ID NO:10, or the complement thereof; position 323 according to SEQ ID NO:11, or the complement thereof; position 326 according to SEQ ID NO:12, or the complement thereof; position 305 according to SEQ ID NO:13, or the complement thereof; or position 170 according to SEQ ID NO:14, or the complement thereof;
wherein when the sequenced portion of the GPAM mRNA molecule in the biological sample comprises a guanine at a position corresponding: to position 327 according to SEQ ID NO:9, position 291 according to SEQ ID NO:10, position 323 according to SEQ ID NO:11, position 326 according to SEQ ID NO:12, position 305 according to SEQ ID NO:13, or position 170 according to SEQ ID NO:14, then the GPAM mRNA molecule in the biological sample is a GPAM predicted loss-of-function variant mRNA molecule.

44. The method according to claim 41, wherein the sequence analysis comprises sequencing at least a portion of the nucleotide sequence of the GPAM cDNA molecule produced from an mRNA molecule in the biological sample, wherein the sequenced portion comprises a position corresponding to: position 327 according to SEQ ID NO:21, or the complement thereof; position 291 according to SEQ ID NO:22, or the complement thereof; position 323 according to SEQ ID NO:23, or the complement thereof; position 326 according to SEQ ID NO:24, or the complement thereof; position 305 according to SEQ ID NO:25, or the complement thereof; or position 170 according to SEQ ID NO:26, or the complement thereof;
wherein when the sequenced portion of the GPAM cDNA molecule in the biological sample comprises a guanine at a position corresponding to: position 327 according to SEQ ID NO:21, position 291 according to SEQ ID NO:22, position 323 according to SEQ ID NO:23, position 326 according to SEQ ID NO:24, position 305 according to SEQ ID NO:25, or position 170 according to SEQ ID NO:26, then the GPAM cDNA molecule produced from an mRNA molecule in the biological sample is a GPAM predicted loss-of-function variant cDNA molecule.

45-56. (canceled)

57. The method according to claim 37, wherein the GPAM inhibitor comprises an antisense nucleic acid molecule, a small interfering RNA (siRNA), or a short hairpin RNA (shRNA) that hybridizes to a GPAM mRNA.

58. The method according to claim 37, wherein the GPAM inhibitor comprises a Cas protein and guide RNA (gRNA) that hybridizes to a gRNA recognition sequence within a GPAM genomic nucleic acid molecule.

59-64. (canceled)

65. The method according to claim 37, wherein the GPAM inhibitor comprises a small molecule.

66. A method of identifying a subject having an increased risk for developing liver disease, wherein the method comprises:
determining or having determined the presence or absence of a glycerol-3-phosphate acyltransferase (GPAM) predicted loss-of-function variant nucleic acid molecule encoding a human GPAM polypeptide in a biological sample obtained from the subject;
wherein:
when the subject is GPAM reference, then the subject has an increased risk for developing liver disease; and
when the subject is heterozygous or homozygous for a GPAM predicted loss-of-function variant, then the subject has a decreased risk for developing liver disease.

67-95. (canceled)

96. The method according to claim 37, wherein the fatty liver disease comprises alcoholic fatty liver disease (AFLD) or nonalcoholic fatty liver disease (NAFLD).

97. The method according to claim 65, wherein the small molecule comprises FSG67.

98. The method according to claim 65, wherein the small molecule comprises a substituted or unsubstituted benzoic acid, a substituted or unsubstituted benzoic acid derivative, a substituted or unsubstituted 7-aminoindole derivative, a substituted or unsubstituted thiophene, a substituted or unsubstituted thiolactone, a substituted or unsubstituted phosphonate, a substituted or unsubstituted phenyl, a substituted or unsubstituted 2-, 3-, or 4-(alkanesulfonamido)benzoic acid, or a substituted or unsubstituted 2-, 3-, or 4-(alkanesulfonamido)benzoic phosphonic acid.

99. The method according to claim 98, wherein the substituted or unsubstituted benzoic acid derivative comprises a substituted or unsubstituted 2-(alkanesulfonamido)benzoic acid, 4-([1,1′-biphenyl]-4-carbonyl)-2-(octanesulfonamido)benzoic acid), or 2-(nonylsulfonamido)benzoic acid.

100. The method according to claim 98, wherein the substituted or unsubstituted 7-aminoindole derivative comprises a 7-amino-5-cyanoindole.
